(19) United States
(12) Patent Application Publication
(10) Pub. No.: US 2025/0014761 A1
(43) Pub. Date: Jan. 9, 2025

(54) BIOMARKER SIGNATURES INDICATIVE OF EARLY STAGES OF CANCER

(71) Applicant: Serum Detect, Inc., Newton, MA (US)

(72) Inventors: Roman Yelensky, Newton, MA (US); Michelle Nahas, Cambridge, MA (US); Yilong Li, Cambridge, MA (US)

(21) Appl. No.: 18/893,253

(22) Filed: Sep. 23, 2024

Related U.S. Application Data
(63) Continuation of application No. PCT/US2023/016065, filed on Mar. 23, 2023.
(60) Provisional application No. 63/322,746, filed on Mar. 23, 2022.

(51) Int. Cl. G16H 50/30; G01N 33/573; G01N 33/574; G01N 33/68; G16H 10/40

(52) U.S. Cl. CPC G16H 50/30; G01N 33/573; G01N 33/57423; G01N 33/57484; G01N 33/6869; G01N 33/6893; G16H 10/40; G01N 2333/495; G01N 2333/5412; G01N 2333/7158; G01N 2333/96486

(57) ABSTRACT

Predictive models are deployed to generate cancer predictions (e.g., presence or absence of cancer) for subjects of interest. Predictive models analyze expression values of two or more biomarkers and can identify, with high sensitivity and specificity, subjects with a presence of cancer.

### CROSS REFERENCE TO RELATED APPLICATIONS

This application claims the benefit of and priority to U.S. Provisional Patent Application No. 63/322,746 filed Mar. 23, 2022, the entire disclosure of which is hereby incorporated by reference in its entirety for all purposes.

### BACKGROUND

Cancer remains a difficult disease to treat, due to the fact that by the time symptoms present in an individual, the cancer has often progressed to an incurable stage. Yet, identifying individuals at an early enough stage for curative treatment is still elusive. Thus, there is a need for practical methods that can rapidly and affordably identify individuals that are likely to have a presence of cancer.

### SUMMARY

Disclosed herein are methods, systems, non-transitory computer readable media, and kits for generating cancer predictions (e.g., predicting presence or absence of cancer, such as early stages of cancer) for subjects of interest. In various embodiments, methods for generating cancer predictions involve the implementation of a predictive model that analyzes expression values of two or more biomarkers, such as two or more biomarkers detailed in Table 2, Table 3, Table 4, or Table 5. Biomarker panels disclosed herein are useful for analyzing biomarker signatures that enable detection of cancer e.g., at its early stages.

Disclosed herein is a method for predicting presence or absence of cancer in a subject comprises: obtaining or having obtained a dataset comprising expression levels of a plurality of biomarkers from the subject, wherein the plurality of biomarkers comprise two or more biomarkers of IL6, TGFA, S100A12, OSM, TFPI2, LSP1, MDK, CXCL9, CLEC4D, HGF, VWA1, CEACAM5, MMP12, KRT19, CASP8, WFDC2, ALPP, and PLAUR; and generating a prediction of presence or absence of the cancer in the subject by applying a predictive model to the expression levels of the plurality of biomarkers. Also disclosed herein is a method for predicting presence or absence of cancer in a subject comprises: obtaining or having obtained a dataset comprising expression levels of a plurality of biomarkers from the subject, wherein the plurality of biomarkers comprise two or more biomarkers of IL6, TGFA, S100A12, OSM, TFPI2, LSP1, MDK, CXCL9, CLEC4D, HGF, VWA1, CEACAM5, MMP12, KRT19, CASP8, WFDC2, and PLAUR; and generating a prediction of presence or absence of the cancer in the subject by applying a predictive model to the expression levels of the plurality of biomarkers.

In various embodiments, a performance of the predictive model is characterized by an area under the curve (AUC) of at least 0.60, at least 0.61, at least 0.62, at least 0.63, at least 0.64, at least 0.65, at least 0.66, at least 0.67, at least 0.68, at least 0.69, at least 0.70, at least 0.71, at least 0.72, at least 0.73, or at least 0.74. In various embodiments, a performance of the predictive model is characterized by an area under the curve (AUC) of at least 0.60. In various embodiments, a performance of the predictive model is characterized by an area under the curve (AUC) of at least 0.74. In various embodiments, a performance metric of the predictive model is improved in comparison to a model solely incorporating CEACAM5 (e.g., a cancer marker in common use today), with example AUC of 0.62.

In various embodiments, the plurality of biomarkers comprises IL-6 and at least one more biomarker is selected from the group comprising: TGFA, S100A12, OSM, TFPI2, LSP1, MDK, CXCL9, CLEC4D, HGF, VWA1, CEACAM5, MMP12, KRT19, CASP8, WFDC2, and PLAUR. In various embodiments, the plurality of biomarkers comprise a combination of biomarkers as shown in Table 5. In various embodiments, a performance of the predictive model is characterized by an area under the curve (AUC) of at least 0.60. In various embodiments, a performance of the predictive model is characterized by a true positive rate of at least 30% at a false positive rate of 10%.

In various embodiments, the plurality of biomarkers comprises IL-6 and at least one more biomarker is selected from the group comprising: TGFA, S100A12, OSM, TFPI2, LSP1, MDK, CXCL9, HGF, VWA1, CEACAM5, MMP12, KRT19, CASP8, WFDC2, and PLAUR. In various embodiments, the plurality of biomarkers comprise a combination of biomarkers as shown in Table 5. In various embodiments, a performance of the predictive model is characterized by an area under the curve (AUC) of at least 0.72. In various embodiments, a performance of the predictive model is characterized by a true positive rate of at least 30% at a false positive rate of 10%.

In various embodiments, the plurality of biomarkers comprises IL-6 and at least one more biomarker is selected from the group comprising: TGFA, S100A12, OSM, LSP1, MDK, CXCL9, HGF, CEACAM5, MMP12, KRT19, WFDC2, and PLAUR. In various embodiments, the plurality of biomarkers is selected from IL6, LSP1, MDK, MMP12; CEACAM5, IL6, MDK, MMP12, TGFA; HGF, IL6, MDK, MMP12, TGFA; CEACAM5, IL6, MDK, TGFA; IL6, MDK, MMP12, OSM; IL6, MDK, MMP12, TGFA; CEACAM5, IL6, LSP1, MDK, TGFA; HGF, IL6, MDK, MMP12, OSM; HGF, IL6, LSP1, MDK, MMP12; IL6, KRT19, MDK, MMP12, TGFA; HGF, IL6, LSP1, MDK; IL6, LSP1, MDK; IL6, LSP1, MDK, TGFA; IL6, MDK, TGFA; CXCL9, IL6, LSP1, MDK; CEACAM5, IL6, MDK, OSM, TGFA; CEACAM5, HGF, IL6, MDK, TGFA; CEACAM5, IL6, MDK, OSM; CEACAM5, IL6, MDK, MMP12, OSM; HGF, IL6, LSP1, MDK, TGFA; CEACAM5, IL6, LSP1, MDK; CEACAM5, IL6, MDK, S100A12, TGFA; HGF, IL6, LSP1, MDK, OSM; CEACAM5, HGF, IL6, MDK, OSM; IL6, LSP1, MDK, MMP12, TGFA; IL6, MDK, MMP12, OSM, TGFA; CEACAM5, IL6, MDK, TGFA, WFDC2; CXCL9, IL6, LSP1, MDK, MMP12; IL6, LSP1, MDK, MMP12, OSM; IL6, KRT19, LSP1, MDK, TGFA; IL6, LSP1, MDK, TGFA, WFDC2; CEACAM5, IL6, LSP1, MDK, MMP12; CEACAM5, IL6, MDK, PLAUR, TGFA; HGF, IL6, MDK, TGFA; or IL6, MDK, TGFA, WFDC2. In various embodiments, a performance of the predictive model is characterized by an area under the curve (AUC) of at least 0.73. In various embodiments, a performance of the predictive model is characterized by a true positive rate of at least 30% at a false positive rate of 10%.

In various embodiments, the plurality of biomarkers comprises IL-6 and MDK, and at least one more biomarker is selected from the group comprising: MMP12, LSP1, CEACAM5, HGF, OSM, and KRT19. In various embodiments, the plurality of biomarkers is selected from IL6, LSP1, MDK, MMP12; CEACAM5, IL6, MDK, MMP12, TGFA; HGF, IL6, MDK, MMP12, TGFA; CEACAM5, IL6, MDK, TGFA; IL6, MDK, MMP12, OSM; IL6, MDK, MMP12, TGFA; CEACAM5, IL6, LSP1, MDK, TGFA; HGF, IL6, MDK, MMP12, OSM; HGF, IL6, LSP1, MDK, MMP12; or IL6, KRT19, MDK, MMP12, TGFA. In various embodiments, a performance of the predictive model is characterized by an area under the curve (AUC) of at least 0.74. In various embodiments, a performance of the predictive model is characterized by a true positive rate of at least 30% at a false positive rate of 10%.

In various embodiments, the cancer is lung cancer. In various embodiments, the lung cancer is an adenocarcinoma, an adenosquamous cell cancer, a large cell cancer, a neuroendocrine cancer, a non-small cell lung cancer (NSCLC), a small cell cancer, or a squamous cell cancer. In various embodiments, the cancer is an early stage cancer. In various embodiments, the cancer is stage I and/or stage II lung cancer. In various embodiments, the expression levels of the plurality of biomarkers is determined from a test sample obtained from the subject. In various embodiments, the test sample is a blood or serum sample. In various embodiments, the subject is suspected of having an early stage cancer. In various embodiments, the subject is not suspected of having an early stage cancer.

In various embodiments, obtaining or having obtained the dataset comprises performing an assay to determine the expression levels of the plurality of biomarkers. In various embodiments, the assay is a Proximity Extension Assay (PEA), a xMAP Multiplex Assay, a single molecule array (SIMOA) assay, mass spectrometry based protein or peptide assay, or an aptamer-based assay. In various embodiments, performing the assay comprises contacting a test sample with a plurality of reagents comprising antibodies. In various embodiments, the antibodies comprise one of monoclonal and polyclonal antibodies. In various embodiments, the antibodies comprise both monoclonal and polyclonal antibodies.

Additionally disclosed herein is a method for predicting presence or absence of cancer in a subject comprises: at a computer system having one or more processors, and memory storing one or more programs for execution by the one or more processors: obtaining or having obtained a dataset comprising expression levels of a plurality of biomarkers from the subject, wherein the plurality of biomarkers comprise two or more biomarkers of IL6, TGFA, S100A12, OSM, TFPI2, LSP1, MDK, CXCL9, CLEC4D, HGF, VWA1, CEACAM5, MMP12, KRT19, CASP8, WFDC2, and PLAUR; and generating a prediction of presence or absence of the cancer in the subject by applying a predictive model to the expression levels of the plurality of biomarkers.

In various embodiments, a performance of the predictive model is characterized by an area under the curve (AUC) of at least 0.60, at least 0.61, at least 0.62, at least 0.63, at least 0.64, at least 0.65, at least 0.66, at least 0.67, at least 0.68, at least 0.69, at least 0.70, at least 0.71, at least 0.72, at least 0.73, or at least 0.74. In various embodiments, a performance of the predictive model is characterized by an area under the curve (AUC) of at least 0.60. In various embodiments, a performance of the predictive model is characterized by an area under the curve (AUC) of at least 0.74. In various embodiments, a performance metric of the predictive model is improved in comparison to a model solely incorporating CEACAM5 (e.g., a cancer marker in common use today).

In various embodiments, the plurality of biomarkers comprises IL-6 and at least one more biomarker is selected from the group comprising: TGFA, S100A12, OSM, TFPI2, LSP1, MDK, CXCL9, CLEC4D, HGF, VWA1, CEACAM5, MMP12, KRT19, CASP8, WFDC2, and PLAUR. In various embodiments, the plurality of biomarkers comprise a combination of biomarkers as shown in Table 5. In various embodiments, a performance of the predictive model is characterized by an area under the curve (AUC) of at least 0.60. In various embodiments, a performance of the predictive model is characterized by a true positive rate of at least 30% at a false positive rate of 10%.

In various embodiments, the plurality of biomarkers comprises IL-6 and at least one more biomarker is selected from the group comprising: TGFA, S100A12, OSM, TFPI2, LSP1, MDK, CXCL9, HGF, VWA1, CEACAM5, MMP12, KRT19, CASP8, WFDC2, and PLAUR. In various embodiments, the plurality of biomarkers comprise a combination of biomarkers as shown in Table 5. In various embodiments, a performance of the predictive model is characterized by an area under the curve (AUC) of at least 0.72. In various embodiments, a performance of the predictive model is characterized by a true positive rate of at least 30% at a false positive rate of 10%.

In various embodiments, the plurality of biomarkers comprises IL-6 and at least one more biomarker is selected from the group comprising: TGFA, S100A12, OSM, LSP1, MDK, CXCL9, HGF, CEACAM5, MMP12, KRT19, WFDC2, and PLAUR. In various embodiments, the plurality of biomarkers is selected from IL6, LSP1, MDK, MMP12; CEACAM5, IL6, MDK, MMP12, TGFA; HGF, IL6, MDK, MMP12, TGFA; CEACAM5, IL6, MDK, TGFA; IL6, MDK, MMP12, OSM; IL6, MDK, MMP12, TGFA; CEACAM5, IL6, LSP1, MDK, TGFA; HGF, IL6, MDK, MMP12, OSM; HGF, IL6, LSP1, MDK, MMP12; IL6, KRT19, MDK, MMP12, TGFA; HGF, IL6, LSP1, MDK; IL6, LSP1, MDK; IL6, LSP1, MDK, TGFA; IL6, MDK, TGFA; CXCL9, IL6, LSP1, MDK; CEACAM5, IL6, MDK, OSM, TGFA; CEACAM5, HGF, IL6, MDK, TGFA; CEACAM5, IL6, MDK, OSM; CEACAM5, IL6, MDK, MMP12, OSM; HGF, IL6, LSP1, MDK, TGFA; CEACAM5, IL6, LSP1, MDK; CEACAM5, IL6, MDK, S100A12, TGFA; HGF, IL6, LSP1, MDK, OSM; CEACAM5, HGF, IL6, MDK, OSM; IL6, LSP1, MDK, MMP12, TGFA; IL6, MDK, MMP12, OSM, TGFA; CEACAM5, IL6, MDK, TGFA, WFDC2; CXCL9, IL6, LSP1, MDK, MMP12; IL6, LSP1, MDK, MMP12, OSM; IL6, KRT19, LSP1, MDK, TGFA; IL6, LSP1, MDK, TGFA, WFDC2; CEACAM5, IL6, LSP1, MDK, MMP12; CEACAM5, IL6, MDK, PLAUR, TGFA; HGF, IL6, MDK, TGFA; or IL6, MDK, TGFA, WFDC2. In various embodiments, a performance of the predictive model is characterized by an area under the curve (AUC) of at least 0.73. In various embodiments, a performance of the predictive model is characterized by a true positive rate of at least 30% at a false positive rate of 10%.

In various embodiments, the plurality of biomarkers comprises IL-6 and MDK, and at least one more biomarker is selected from the group comprising: MMP12, LSP1, CEACAM5, HGF, OSM, and KRT19. In various embodiments, the plurality of biomarkers is selected from IL6, LSP1, MDK, MMP12; CEACAM5, IL6, MDK, MMP12, TGFA; HGF, IL6, MDK, MMP12, TGFA; CEACAM5, IL6, MDK, TGFA; IL6, MDK, MMP12, OSM; IL6, MDK, MMP12, TGFA; CEACAM5, IL6, LSP1, MDK, TGFA; HGF, IL6, MDK, MMP12, OSM; HGF, IL6, LSP1, MDK, MMP12; or IL6, KRT19, MDK, MMP12, TGFA. In various embodiments, a performance of the predictive model is characterized by an area under the curve (AUC) of at least 0.74. In various embodiments, a performance of the predictive model is characterized by a true positive rate of at least 30% at a false positive rate of 10%.

In various embodiments, the cancer is lung cancer. In various embodiments, the lung cancer is an adenocarcinoma, an adenosquamous cell cancer, a large cell cancer, a neuroendocrine cancer, a non-small cell lung cancer (NSCLC), a small cell cancer, or a squamous cell cancer. In various embodiments, the cancer is an early stage cancer. In various embodiments, the cancer is stage I and/or stage II lung cancer. In various embodiments, the expression levels of the plurality of biomarkers is determined from a test sample obtained from the subject. In various embodiments, the test sample is a blood or serum sample. In various embodiments, the subject is suspected of having an early stage cancer. In various embodiments, the subject is not suspected of having an early stage cancer.

In various embodiments, obtaining or having obtained the dataset comprises performing an assay to determine the expression levels of the plurality of biomarkers. In various embodiments, the assay is a Proximity Extension Assay (PEA), a xMAP Multiplex Assay, a single molecule array (SIMOA) assay, mass spectrometry based protein or peptide assay, or an aptamer-based assay. In various embodiments, performing the assay comprises contacting a test sample with a plurality of reagents comprising antibodies. In various embodiments, the antibodies comprise one of monoclonal and polyclonal antibodies. In various embodiments, the antibodies comprise both monoclonal and polyclonal antibodies.

Additionally disclosed herein is anon-transitory computer readable medium comprises instructions that, when executed by a processor, cause the processor to: obtain a dataset comprising expression levels of a plurality of biomarkers from the subject, wherein the plurality of biomarkers comprise two or more biomarkers of IL6, TGFA, S100A12, OSM, TFPI2, LSP1, MDK, CXCL9, CLEC4D, HGF, VWA1, CEACAM5, MMP12, KRT19, CASP8, WFDC2, and PLAUR; and generating a prediction of presence or absence of the cancer in the subject by applying a predictive model to the expression levels of the plurality of biomarkers.

In various embodiments, a performance of the predictive model is characterized by an area under the curve (AUC) of at least 0.60, at least 0.61, at least 0.62, at least 0.63, at least 0.64, at least 0.65, at least 0.66, at least 0.67, at least 0.68, at least 0.69, at least 0.70, at least 0.71, at least 0.72, at least 0.73, or at least 0.74. In various embodiments, a performance of the predictive model is characterized by an area under the curve (AUC) of at least 0.60. In various embodiments, a performance of the predictive model is characterized by an area under the curve (AUC) of at least 0.74. In various embodiments, a performance metric of the predictive model is improved in comparison to a model solely incorporating CEACAM5.

In various embodiments, the plurality of biomarkers comprises IL-6 and at least one more biomarker is selected from the group comprising: TGFA, S100A12, OSM, TFPI2, LSP1, MDK, CXCL9, CLEC4D, HGF, VWA1, CEACAM5, MMP12, KRT19, CASP8, WFDC2, and PLAUR. In various embodiments, the plurality of biomarkers comprise a combination of biomarkers as shown in Table 5. In various embodiments, a performance of the predictive model is characterized by an area under the curve (AUC) of at least 0.60. In various embodiments, a performance of the predictive model is characterized by a true positive rate of at least 30% at a false positive rate of 10%.

In various embodiments, the plurality of biomarkers comprises IL-6 and at least one more biomarker is selected from the group comprising: TGFA, S100A12, OSM, TFPI2, LSP1, MDK, CXCL9, HGF, VWA1, CEACAM5, MMP12, KRT19, CASP8, WFDC2, and PLAUR. In various embodiments, the plurality of biomarkers comprise a combination of biomarkers as shown in Table 5. In various embodiments, a performance of the predictive model is characterized by an area under the curve (AUC) of at least 0.72. In various embodiments, a performance of the predictive model is characterized by a true positive rate of at least 30% at a false positive rate of 10%.

In various embodiments, the plurality of biomarkers comprises IL-6 and at least one more biomarker is selected from the group comprising: TGFA, S100A12, OSM, LSP1, MDK, CXCL9, HGF, CEACAM5, MMP12, KRT19, WFDC2, and PLAUR. In various embodiments, the plurality of biomarkers is selected from IL6, LSP1, MDK, MMP12; CEACAM5, IL6, MDK, MMP12, TGFA; HGF, IL6, MDK, MMP12, TGFA; CEACAM5, IL6, MDK, TGFA; IL6, MDK, MMP12, OSM; IL6, MDK, MMP12, TGFA; CEACAM5, IL6, LSP1, MDK, TGFA; HGF, IL6, MDK, MMP12, OSM; HGF, IL6, LSP1, MDK, MMP12; IL6, KRT19, MDK, MMP12, TGFA; HGF, IL6, LSP1, MDK; IL6, LSP1, MDK; IL6, LSP1, MDK, TGFA; IL6, MDK, TGFA; CXCL9, IL6, LSP1, MDK; CEACAM5, IL6, MDK, OSM, TGFA; CEACAM5, HGF, IL6, MDK, TGFA; CEACAM5, IL6, MDK, OSM; CEACAM5, IL6, MDK, MMP12, OSM; HGF, IL6, LSP1, MDK, TGFA; CEACAM5, IL6, LSP1, MDK; CEACAM5, IL6, MDK, S100A12, TGFA; HGF, IL6, LSP1, MDK, OSM; CEACAM5, HGF, IL6, MDK, OSM; IL6, LSP1, MDK, MMP12, TGFA; IL6, MDK, MMP12, OSM, TGFA; CEACAM5, IL6, MDK, TGFA, WFDC2; CXCL9, IL6, LSP1, MDK, MMP12; IL6, LSP1, MDK, MMP12, OSM; IL6, KRT19, LSP1, MDK, TGFA; IL6, LSP1, MDK, TGFA, WFDC2; CEACAM5, IL6, LSP1, MDK, MMP12; CEACAM5, IL6, MDK, PLAUR, TGFA; HGF, IL6, MDK, TGFA; or IL6, MDK, TGFA, WFDC2. In various embodiments, a performance of the predictive model is characterized by an area under the curve (AUC) of at least 0.73. In various embodiments, a performance of the predictive model is characterized by a true positive rate of at least 30% at a false positive rate of 10%.

In various embodiments, the plurality of biomarkers comprises IL-6 and MDK, and at least one more biomarker is selected from the group comprising: MMP12, LSP1, CEACAM5, HGF, OSM, and KRT19. In various embodiments, the plurality of biomarkers is selected from IL6, LSP1, MDK, MMP12; CEACAM5, IL6, MDK, MMP12, TGFA; HGF, IL6, MDK, MMP12, TGFA; CEACAM5, IL6, MDK, TGFA; IL6, MDK, MMP12, OSM; IL6, MDK, MMP12, TGFA; CEACAM5, IL6, LSP1, MDK, TGFA; HGF, IL6, MDK, MMP12, OSM; HGF, IL6, LSP1, MDK, MMP12; or IL6, KRT19, MDK, MMP12, TGFA. In various embodiments, a performance of the predictive model is characterized by an area under the curve (AUC) of at least 0.74. In various embodiments, a performance of the predictive model is characterized by a true positive rate of at least 30% at a false positive rate of 10%.

In various embodiments, the cancer is lung cancer. In various embodiments, the lung cancer is an adenocarcinoma, an adenosquamous cell cancer, a large cell cancer, a neuroendocrine cancer, a non-small cell lung cancer (NSCLC), a small cell cancer, or a squamous cell cancer. In various embodiments, the cancer is an early stage cancer. In various embodiments, the cancer is stage I and/or stage II lung cancer. In various embodiments, the expression levels of the plurality of biomarkers is determined from a test sample obtained from the subject. In various embodiments, the test sample is a blood or serum sample. In various embodiments, the subject is suspected of having an early stage cancer. In various embodiments, the subject is not suspected of having an early stage cancer.

Additionally disclosed herein is a system comprises: a set of reagents used for determining expression levels for a plurality of biomarkers from a test sample from the subject, wherein the plurality of biomarkers comprise two or more biomarkers of IL6, TGFA, S100A12, OSM, TFPI2, LSP1, MDK, CXCL9, CLEC4D, HGF, VWA1, CEACAM5, MMP12, KRT19, CASP8, WFDC2, and PLAUR; an apparatus configured to receive a mixture of one or more reagents in the set and the test sample and to measure the expression levels for the biomarkers from the test sample; and a computer system communicatively coupled to the apparatus to obtain a dataset comprising the expression levels for the plurality of biomarkers from the test sample and to generate a presence or absence of cancer in the subject by applying a predictive model to the expression levels of the plurality of biomarkers.

In various embodiments, a performance of the predictive model is characterized by an area under the curve (AUC) of at least 0.60, at least 0.61, at least 0.62, at least 0.63, at least 0.64, at least 0.65, at least 0.66, at least 0.67, at least 0.68, at least 0.69, at least 0.70, at least 0.71, at least 0.72, at least 0.73, or at least 0.74. In various embodiments, a performance of the predictive model is characterized by an area under the curve (AUC) of at least 0.60. In various embodiments, a performance of the predictive model is characterized by an area under the curve (AUC) of at least 0.74. In various embodiments, a performance metric of the predictive model is improved in comparison to a model solely incorporating CEACAM5.

In various embodiments, the plurality of biomarkers comprises IL-6 and at least one more biomarker is selected from the group comprising: TGFA, S100A12, OSM, TFPI2, LSP1, MDK, CXCL9, CLEC4D, HGF, VWA1, CEACAM5, MMP12, KRT19, CASP8, WFDC2, and PLAUR. In various embodiments, the plurality of biomarkers comprise a combination of biomarkers as shown in Table 5. In various embodiments, a performance of the predictive model is characterized by an area under the curve (AUC) of at least 0.60. In various embodiments, a performance of the predictive model is characterized by a true positive rate of at least 30% at a false positive rate of 10%.

In various embodiments, the plurality of biomarkers comprises IL-6 and at least one more biomarker is selected from the group comprising: TGFA, S100A12, OSM, TFPI2, LSP1, MDK, CXCL9, HGF, VWA1, CEACAM5, MMP12, KRT19, CASP8, WFDC2, and PLAUR. In various embodiments, the plurality of biomarkers comprise a combination of biomarkers as shown in Table 5. In various embodiments, a performance of the predictive model is characterized by an area under the curve (AUC) of at least 0.72. In various embodiments, a performance of the predictive model is characterized by a true positive rate of at least 30% at a false positive rate of 10%.

In various embodiments, the plurality of biomarkers comprises IL-6 and at least one more biomarker is selected from the group comprising: TGFA, S100A12, OSM, LSP1, MDK, CXCL9, HGF, CEACAM5, MMP12, KRT19, WFDC2, and PLAUR. In various embodiments, the plurality of biomarkers is selected from IL6, LSP1, MDK, MMP12; CEACAM5, IL6, MDK, MMP12, TGFA; HGF, IL6, MDK, MMP12, TGFA; CEACAM5, IL6, MDK, TGFA; IL6, MDK, MMP12, OSM; IL6, MDK, MMP12, TGFA; CEACAM5, IL6, LSP1, MDK, TGFA; HGF, IL6, MDK, MMP12, OSM; HGF, IL6, LSP1, MDK, MMP12; IL6, KRT19, MDK, MMP12, TGFA; HGF, IL6, LSP1, MDK; IL6, LSP1, MDK; IL6, LSP1, MDK, TGFA; IL6, MDK, TGFA; CXCL9, IL6, LSP1, MDK; CEACAM5, IL6, MDK, OSM, TGFA; CEACAM5, HGF, IL6, MDK, TGFA; CEACAM5, IL6, MDK, OSM; CEACAM5, IL6, MDK, MMP12, OSM; HGF, IL6, LSP1, MDK, TGFA; CEACAM5, IL6, LSP1, MDK; CEACAM5, IL6, MDK, S100A12, TGFA; HGF, IL6, LSP1, MDK, OSM; CEACAM5, HGF, IL6, MDK, OSM; IL6, LSP1, MDK, MMP12, TGFA; IL6, MDK, MMP12, OSM, TGFA; CEACAM5, IL6, MDK, TGFA, WFDC2; CXCL9, IL6, LSP1, MDK, MMP12; IL6, LSP1, MDK, MMP12, OSM; IL6, KRT19, LSP1, MDK, TGFA; IL6, LSP1, MDK, TGFA, WFDC2; CEACAM5, IL6, LSP1, MDK, MMP12; CEACAM5, IL6, MDK, PLAUR, TGFA; HGF, IL6, MDK, TGFA; or IL6, MDK, TGFA, WFDC2. In various embodiments, a performance of the predictive model is characterized by an area under the curve (AUC) of at least 0.73. In various embodiments, a performance of the predictive model is characterized by a true positive rate of at least 30% at a false positive rate of 10%.

In various embodiments, the plurality of biomarkers comprises IL-6 and MDK, and at least one more biomarker is selected from the group comprising: MMP12, LSP1, CEACAM5, HGF, OSM, and KRT19. In various embodiments, the plurality of biomarkers is selected from IL6, LSP1, MDK, MMP12; CEACAM5, IL6, MDK, MMP12, TGFA; HGF, IL6, MDK, MMP12, TGFA; CEACAM5, IL6, MDK, TGFA; IL6, MDK, MMP12, OSM; IL6, MDK, MMP12, TGFA; CEACAM5, IL6, LSP1, MDK, TGFA; HGF, IL6, MDK, MMP12, OSM; HGF, IL6, LSP1, MDK, MMP12; or IL6, KRT19, MDK, MMP12, TGFA. In various embodiments, a performance of the predictive model is characterized by an area under the curve (AUC) of at least 0.74. In various embodiments, a performance of the predictive model is characterized by a true positive rate of at least 30% at a false positive rate of 10%.

In various embodiments, the cancer is lung cancer. In various embodiments, the lung cancer is an adenocarcinoma, an adenosquamous cell cancer, a large cell cancer, a neuroendocrine cancer, a non-small cell lung cancer (NSCLC), a small cell cancer, or a squamous cell cancer. In various embodiments, the cancer is an early stage cancer. In various embodiments, the cancer is stage I and/or stage II lung cancer. In various embodiments, the expression levels of the plurality of biomarkers is determined from a test sample obtained from the subject. In various embodiments, the test sample is a blood or serum sample. In various embodiments, the subject is suspected of having an early stage cancer. In various embodiments, the subject is not suspected of having an early stage cancer.

Additionally disclosed herein is a kit for predicting presence or absence of cancer in a subject, comprises: a set of reagents for determining expression levels for a plurality of biomarkers from a test sample from the subject, wherein the plurality of biomarkers comprise two or more biomarkers of IL6, TGFA, S100A12, OSM, TFPI2, LSP1, MDK, CXCL9, CLEC4D, HGF, VWA1, CEACAM5, MMP12, KRT19, CASP8, WFDC2, and PLAUR; and instructions for using the set of reagents to determine the expression levels of the plurality of biomarkers from the test sample and to generate a prediction of presence or absence of cancer in the subject by applying a predictive model to the expression levels of the plurality of biomarkers.

In various embodiments, a performance of the predictive model is characterized by an area under the curve (AUC) of at least 0.60, at least 0.61, at least 0.62, at least 0.63, at least 0.64, at least 0.65, at least 0.66, at least 0.67, at least 0.68, at least 0.69, at least 0.70, at least 0.71, at least 0.72, at least 0.73, or at least 0.74. In various embodiments, a performance of the predictive model is characterized by an area under the curve (AUC) of at least 0.60. In various embodiments, a performance of the predictive model is characterized by an area under the curve (AUC) of at least 0.74. In various embodiments, a performance metric of the predictive model is improved in comparison to a model solely incorporating CEACAM5 (e.g., a cancer marker in common use today).

In various embodiments, the plurality of biomarkers comprises IL-6 and at least one more biomarker is selected from the group comprising: TGFA, S100A12, OSM, TFPI2, LSP1, MDK, CXCL9, CLEC4D, HGF, VWA1, CEACAM5, MMP12, KRT19, CASP8, WFDC2, and PLAUR. In various embodiments, the plurality of biomarkers comprise a combination of biomarkers as shown in Table 5. In various embodiments, a performance of the predictive model is characterized by an area under the curve (AUC) of at least 0.60. In various embodiments, a performance of the predictive model is characterized by a true positive rate of at least 30% at a false positive rate of 10%.

In various embodiments, the plurality of biomarkers comprises IL-6 and at least one more biomarker is selected from the group comprising: TGFA, S100A12, OSM, TFPI2, LSP1, MDK, CXCL9, HGF, VWA1, CEACAM5, MMP12, KRT19, CASP8, WFDC2, and PLAUR. In various embodiments, the plurality of biomarkers comprise a combination of biomarkers as shown in Table 5. In various embodiments, a performance of the predictive model is characterized by an area under the curve (AUC) of at least 0.72. In various embodiments, a performance of the predictive model is characterized by a true positive rate of at least 30% at a false positive rate of 10%.

In various embodiments, the plurality of biomarkers comprises IL-6 and at least one more biomarker is selected from the group comprising: TGFA, S100A12, OSM, LSP1, MDK, CXCL9, HGF, CEACAM5, MMP12, KRT19, WFDC2, and PLAUR. In various embodiments, the plurality of biomarkers is selected from IL6, LSP1, MDK, MMP12; CEACAM5, IL6, MDK, MMP12, TGFA; HGF, IL6, MDK, MMP12, TGFA; CEACAM5, IL6, MDK, TGFA; IL6, MDK, MMP12, OSM; IL6, MDK, MMP12, TGFA; CEACAM5, IL6, LSP1, MDK, TGFA; HGF, IL6, MDK, MMP12, OSM; HGF, IL6, LSP1, MDK, MMP12; IL6, KRT19, MDK, MMP12, TGFA; HGF, IL6, LSP1, MDK; IL6, LSP1, MDK; IL6, LSP1, MDK, TGFA; IL6, MDK, TGFA; CXCL9, IL6, LSP1, MDK; CEACAM5, IL6, MDK, OSM, TGFA; CEACAM5, HGF, IL6, MDK, TGFA; CEACAM5, IL6, MDK, OSM; CEACAM5, IL6, MDK, MMP12, OSM; HGF, IL6, LSP1, MDK, TGFA; CEACAM5, IL6, LSP1, MDK; CEACAM5, IL6, MDK, S100A12, TGFA; HGF, IL6, LSP1, MDK, OSM; CEACAM5, HGF, IL6, MDK, OSM; IL6, LSP1, MDK, MMP12, TGFA; IL6, MDK, MMP12, OSM, TGFA; CEACAM5, IL6, MDK, TGFA, WFDC2; CXCL9, IL6, LSP1, MDK, MMP12; IL6, LSP1, MDK, MMP12, OSM; IL6, KRT19, LSP1, MDK, TGFA; IL6, LSP1, MDK, TGFA, WFDC2; CEACAM5, IL6, LSP1, MDK, MMP12; CEACAM5, IL6, MDK, PLAUR, TGFA; HGF, IL6, MDK, TGFA; or IL6, MDK, TGFA, WFDC2. In various embodiments, a performance of the predictive model is characterized by an area under the curve (AUC) of at least 0.73. In various embodiments, a performance of the predictive model is characterized by a true positive rate of at least 30% at a false positive rate of 10%.

In various embodiments, the plurality of biomarkers comprises IL-6 and MDK, and at least one more biomarker is selected from the group comprising: MMP12, LSP1, CEACAM5, HGF, OSM, and KRT19. In various embodiments, the plurality of biomarkers is selected from IL6, LSP1, MDK, MMP12; CEACAM5, IL6, MDK, MMP12, TGFA; HGF, IL6, MDK, MMP12, TGFA; CEACAM5, IL6, MDK, TGFA; IL6, MDK, MMP12, OSM; IL6, MDK, MMP12, TGFA; CEACAM5, IL6, LSP1, MDK, TGFA; HGF, IL6, MDK, MMP12, OSM; HGF, IL6, LSP1, MDK, MMP12; or IL6, KRT19, MDK, MMP12, TGFA. In various embodiments, a performance of the predictive model is characterized by an area under the curve (AUC) of at least 0.74. In various embodiments, a performance of the predictive model is characterized by a true positive rate of at least 30% at a false positive rate of 10%.

In various embodiments, the cancer is lung cancer. In various embodiments, the lung cancer is an adenocarcinoma, an adenosquamous cell cancer, a large cell cancer, a neuroendocrine cancer, a non-small cell lung cancer (NSCLC), a small cell cancer, or a squamous cell cancer. In various embodiments, the cancer is an early stage cancer. In various embodiments, the cancer is stage I and/or stage II lung cancer. In various embodiments, the expression levels of the plurality of biomarkers is determined from a test sample obtained from the subject. In various embodiments, the test sample is a blood or serum sample. In various embodiments, the subject is suspected of having an early stage cancer. In various embodiments, the subject is not suspected of having an early stage cancer.

In various embodiments, the set of reagents is used to perform an assay to determine the expression levels of the plurality of biomarkers. In various embodiments, wherein the assay is a Proximity Extension Assay (PEA), a xMAP Multiplex Assay, a single molecule array (SIMOA) assay, mass spectrometry based protein or peptide assay, or an aptamer-based assay. In various embodiments, performing the assay comprises contacting a test sample with a plurality of reagents comprising antibodies. In various embodiments, the antibodies comprise one of monoclonal and polyclonal antibodies. In various embodiments, the antibodies comprise both monoclonal and polyclonal antibodies.

### BRIEF DESCRIPTION OF THE DRAWINGS

These and other features, aspects, and advantages of the present invention will become better understood with regard to the following description and accompanying drawings.

FIG. 1A depicts an overview of an environment for generating a cancer prediction in a subject via a cancer prediction system, in accordance with an embodiment.

FIG. 1B is an example block diagram of the cancer prediction system, in accordance with an embodiment.

FIG. 2 depicts a flow diagram for predicting cancer in a subject, in accordance with an embodiment.

FIG. 3 illustrates an example computer for implementing the entities shown in FIGS. 1A, 1B, and 2.

FIG. 4 shows univariate analyses of individual biomarkers for distinguishing cancer versus non-cancer groups.

FIG. 5 shows performance of models incorporating various biomarker combinations for predicting presence or absence of cancer (e.g., different stages of cancer) in the form of a receiver operating curve (ROC).

FIG. 6 illustrates analysis of blood from 110 subjects diagnosed with lung cancer, and 125 subjects without lung cancer (control), enriched for older individuals with a history of smoking.

FIG. 7 illustrates disease stage (top panel) and subtype (bottom panel) analyzed from a cohort of blood samples from 110 patients diagnosed with lung cancer.

### DETAILED DESCRIPTION

### I. Definitions

Terms used in the claims and specification are defined as set forth below unless otherwise specified.

The term “subject” encompasses a cell, tissue, or organism, human or non-human, whether in vivo, ex vivo, or in vitro, male or female.

The term “mammal” encompasses both humans and non-humans and includes but is not limited to humans, non-human primates, canines, felines, murines, bovines, equines, and porcines.

The term “sample” can include a single cell or multiple cells or fragments of cells or an aliquot of body fluid, such as a blood sample, taken from a subject, by means including venipuncture, excretion, ejaculation, massage, biopsy, needle aspirate, lavage sample, scraping, surgical incision, or intervention or other means known in the art. Examples of an aliquot of body fluid include amniotic fluid, aqueous humor, bile, lymph, breast milk, interstitial fluid, blood, blood plasma, cerumen (earwax), Cowper's fluid (pre-ejaculatory fluid), chyle, chyme, female ejaculate, menses, mucus, saliva, urine, vomit, tears, vaginal lubrication, sweat, serum, semen, sebum, pus, pleural fluid, cerebrospinal fluid, synovial fluid, intracellular fluid, and vitreous humour.

The terms “marker,” “markers,” “biomarker,” and “biomarkers” encompass, without limitation, lipids, lipoproteins, proteins, cytokines, chemokines, growth factors, peptides, nucleic acids, genes, and oligonucleotides, together with their related complexes, metabolites, mutations, variants, polymorphisms, modifications, fragments, subunits, degradation products, elements, and other analytes or sample-derived measures. A marker can also include mutated proteins, mutated nucleic acids, variations in copy numbers, and/or transcript variants, in circumstances in which such mutations, variations in copy number and/or transcript variants are useful for generating a predictive model, or are useful in predictive models developed using related markers (e.g., non-mutated versions of the proteins or nucleic acids, alternative transcripts, etc.).

The term “antibody” is used in the broadest sense and specifically covers monoclonal antibodies (including full length monoclonal antibodies), polyclonal antibodies, multispecific antibodies (e.g., bispecific antibodies), and antibody fragments that are antigen-binding so long as they exhibit the desired biological activity, e.g., an antibody or an antigen-binding fragment thereof.

“Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”).

The term “biomarker panel” refers to a set biomarkers that are informative for generating a cancer prediction. For example, expression levels of the set of biomarkers in the biomarker panel can be informative for generating a cancer prediction. In various embodiments, a biomarker panel can include two, three, four, five, six, seven, eight, nine, ten eleven, twelve, thirteen, fourteen, fifteen, sixteen, seventeen, eighteen, nineteen, twenty, twenty one, twenty two, twenty three, twenty four, or twenty five biomarkers.

The term “obtaining a dataset associated with a sample” encompasses obtaining a set of data determined from at least one sample. Obtaining a dataset encompasses obtaining a sample and processing the sample to experimentally determine the data. The phrase also encompasses receiving a set of data, e.g., from a third party that has processed the sample to experimentally determine the dataset. Additionally, the phrase encompasses mining data from at least one database or at least one publication or a combination of databases and publications. A dataset can be obtained by one of skill in the art via a variety of known ways including stored on a storage memory.

It must be noted that, as used in the specification, the singular forms “a,” “an” and “the” include plural referents unless the context clearly dictates otherwise.

### II. Overview

Predictive models, as disclosed herein, are useful for distinguishing subjects having a presence or absence of cancer, such as early stage cancer or non-early stage cancer. Example early stage cancer includes stage I and/or stage II cancer. In comparison, non-early stage cancer (e.g., late stage cancer) includes stage III and/or stage IV cancer. In particular embodiments, the early stage cancer is an early stage lung cancer. In particular embodiments, for a subject of interest, predictive models analyze the expression values of two or more biomarkers of a biomarker panel to generate a cancer prediction (e.g., a prediction of a presence or absence of early stage cancer or non-early stage cancer in the subject of interest).

In various embodiments, predictive models disclosed herein can be trained to achieve high sensitivities. Therefore, such high sensitivity predictive models can correctly classify subjects of interest that have a presence of early stage cancer or non-early stage cancer. Such predictive models that achieve high sensitivities may be useful as a general screening tool for identifying subjects of interest who are candidates for undergoing additional analysis (e.g., additional molecular analysis of blood specimens, additional image scanning such as PET or CT scan, or a tissue biopsy) to confirm the results of the predictive models. Put another way, the disclosed predictive models can serve as a high sensitivity, lower specificity screen that identifies a portion of subjects who are candidates for undergoing additional analysis (e.g., higher specificity analysis). This ensures that the high sensitivity, lower specificity screen, which is often cheaper to implement, can be used to analyze a larger number of subjects whereas the additional, higher specificity analysis, which is often more expensive to implement, can be used to analyze the subset of subjects passing the first screen.

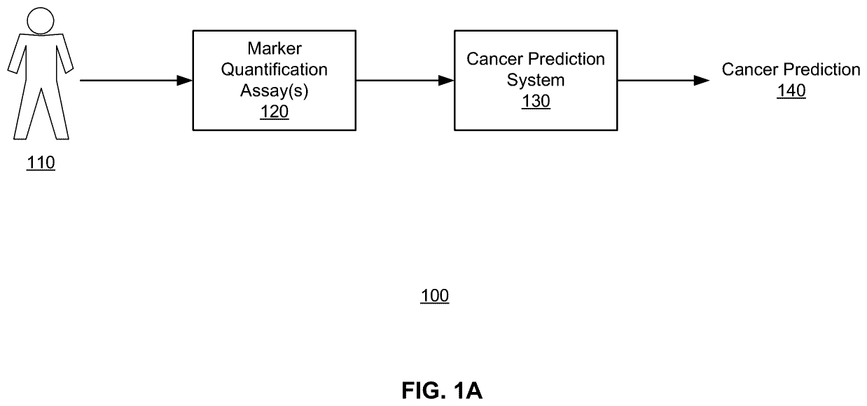

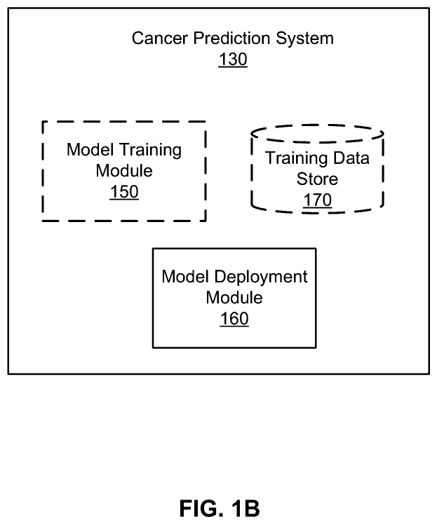

FIG. 1A depicts an overview of a system environment 100 for generating a cancer prediction in a subject via a cancer prediction system 130, in accordance with an embodiment. The system environment 100 provides context in order to introduce a marker quantification assay 120 and a cancer prediction system 130.

In various embodiments, a test sample is obtained from the subject 110. The sample can be obtained by the individual or by a third party, e.g., a medical professional. Examples of medical professionals include physicians, emergency medical technicians, nurses, first responders, psychologists, phlebotomist, medical physics personnel, nurse practitioners, surgeons, dentists, and any other obvious medical professional as would be known to one skilled in the art.

In various embodiments, the subject 110 is suspected of having an early stage cancer or non-early stage cancer. For example, the subject 110 may have exhibited symptoms of early stage cancer or non-early stage cancer. In various embodiments, the subject is not suspected of having an early stage cancer or non-early stage cancer. For example, the subject 110 may be undergoing a standard examination and a test sample is obtained from the subject 110 during the standard examination.

The test sample is tested to determine expression values of one or more markers by performing the marker quantification assay 120. The marker quantification assay 120 determines quantitative expression values of one or more biomarkers from the test sample. The marker quantification assay 120 may be an immunoassay, such as a multi-plex immunoassay, examples of which are described in further detail below. The quantified expression values of the biomarkers are provided to the cancer prediction system 130.

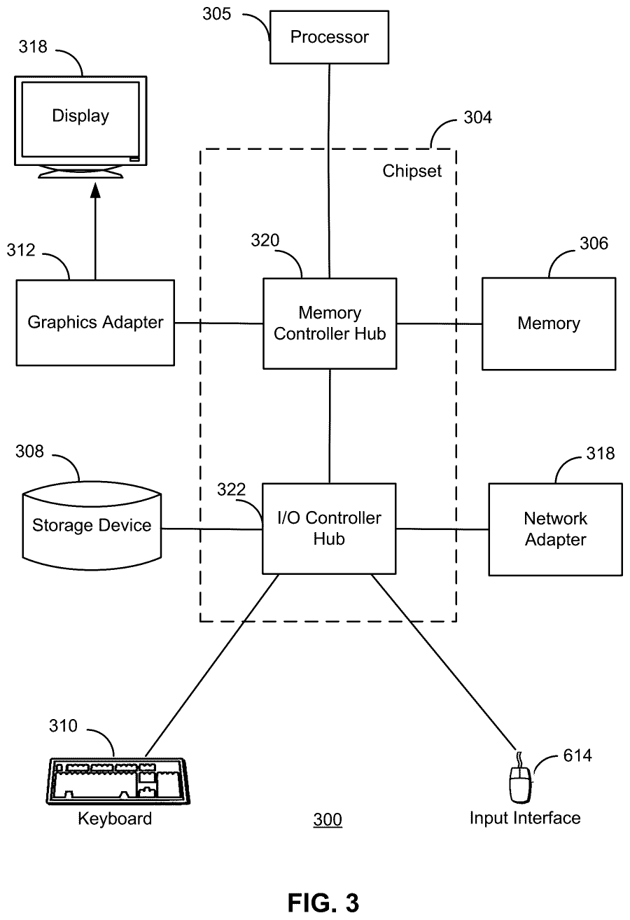

Generally, the cancer prediction system 130 includes one or more computers, embodied as a computer system 300 as discussed below with respect to FIG. 3. Therefore, in various embodiments, the steps described in reference to the cancer prediction system 130 are performed in silico. The cancer prediction system 130 analyzes the received biomarker expression values from the marker quantification assay 120 to generate a cancer prediction 140 (e.g., a presence or absence of cancer) for the subject 110.

In various embodiments, the marker quantification assay 120 and the cancer prediction system 130 can be employed by different parties. For example, a first party performs the marker quantification assay 120 which then provides the results to a second party which deploys the cancer prediction system 130. For example, the first party may be a clinical laboratory that obtains test samples from subjects 110 and performs the assay 120 on the test samples. The second party receives the expression values of biomarkers resulting from the performed assay 120 and analyzes the expression values using the cancer prediction system 130.

FIG. 1B is an example block diagram of the cancer prediction system 130, in accordance with an embodiment. Specifically, the cancer prediction system 130 may include a model training module 150, a model deployment module 160, and a training data store 170.

The components of the cancer prediction system 130 are hereafter described in reference to two phases: 1) a training phase and 2) a deployment phase. More specifically, the training phase refers to the building and training of one or more predictive models based on training data that includes quantitative expression values of biomarkers obtained from individuals that are known to have a presence or absence of cancer. Therefore, during the deployment phase, the predictive model is applied to quantitative biomarker expression values from a test sample obtained from a subject of interest to generate a cancer prediction for the subject of interest.

In some embodiments, the components of the cancer prediction system 130 are applied during one of the training phase and the deployment phase. For example, the model training module 150 and training data store 170 (indicated by the dotted lines in FIG. 1B) are applied during the training phase whereas the model deployment module 160 is applied during the deployment phase. In various embodiments, the components of the cancer prediction system 130 can be performed by different parties depending on whether the components are applied during the training phase or the deployment phase. In such scenarios, the training and deployment of the predictive model are performed by different parties. For example, the model training module 150 and training data store 170 applied during the training phase can be employed by a first party (e.g., to train a predictive model) and the model deployment module 160 applied during the deployment phase can be performed by a second party (e.g., to deploy the predictive model).

### III. Predictive Model

### II.A. Training a Predictive Model

During the training phase, the model training module 150 trains one or more predictive models using training data comprising expression values of biomarkers. In various embodiments, the model training module 150 generates the training data comprising expression values of biomarkers by analyzing biomarker expression values in test samples from individuals known to have a presence or absence of cancer. In various embodiments, the model training module 150 obtains the training data comprising expression values of biomarkers from a third party. The third party may have analyzed test samples to determine the biomarker expression values.

In various embodiments, the training data further comprises reference ground truth values that indicate a cancer status (e.g., presence or absence of cancer) in an individual from whom the expression values of biomarkers were obtained. Example reference ground truth values can be a binary value (e.g., “0” indicating absence of cancer and “1” indicating presence of cancer) or continuous values. Thus, over training iterations, the predictive model is trained (e.g., the parameters are tuned) to minimize a prediction error between a cancer prediction (e.g., presence or absence of cancer) and the reference ground truth values. In various embodiments, the prediction error is calculated based on a loss function, examples of which include a L1 regularization (Lasso Regression) loss function, a L2 regularization (Ridge Regression) loss function, or a combination of L1 and L2 regularization (ElasticNet).

In some embodiments, the model training module 150 retrieves the training data from the training data store 170 and randomly partitions the training data into a training set and a test set. As an example, 80% of the training data may be partitioned into the training set and the other 20% can be partitioned into the test set. Other proportions of training set and test set may be implemented. As such, the training set is used to train predictive models whereas the test set is used to validate the predictive models.

In various embodiments, the predictive model is any one of a regression model (e.g., linear regression, logistic regression, or polynomial regression), decision tree, random forest, support vector machine, Naïve Bayes model, k-means cluster, or neural network (e.g., feed-forward networks, convolutional neural networks (CNN), deep neural networks (DNN), autoencoder neural networks, generative adversarial networks, or recurrent networks (e.g., long short-term memory networks (LSTM), bi-directional recurrent networks, deep bi-directional recurrent networks), or any combination thereof.

The predictive model can be trained using a machine learning implemented method, such as any one of a linear regression algorithm, logistic regression algorithm, decision tree algorithm, support vector machine classification, Naïve Bayes classification, K-Nearest Neighbor classification, random forest algorithm, deep learning algorithm, gradient boosting algorithm, and dimensionality reduction techniques such as manifold learning, principal component analysis, factor analysis, autoencoder regularization, and independent component analysis, or combinations thereof. In various embodiments, the predictive model is trained using supervised learning algorithms, unsupervised learning algorithms, semi-supervised learning algorithms (e.g., partial supervision), weak supervision, transfer, multi-task learning, or any combination thereof.

In various embodiments, the predictive model has one or more parameters, such as hyperparameters or model parameters. Hyperparameters are generally established prior to training. Examples of hyperparameters include the learning rate, depth or leaves of a decision tree, number of hidden layers in a deep neural network, number of clusters in a k-means cluster, penalty in a regression model, and a regularization parameter associated with a cost function. Model parameters are generally adjusted during training. Examples of model parameters include weights associated with nodes in layers of neural network, support vectors in a support vector machine, and coefficients in a regression model. The model parameters of the predictive model are trained (e.g., adjusted) using the training data to improve the predictive capacity of the predictive model.

In various embodiment, the model training module 150 performs a feature selection process to identify the set of biomarkers to be included in the biomarker panel. For example, the model training module 150 performs a sequential forward feature selection based on the expression values of the biomarkers and their importance in predicting the particular output (e.g., presence or absence of cancer). For example, biomarkers that are determined to be highly correlated with a presence or absence of cancer would be deemed highly important are therefore likely to be included in the biomarker panel in comparison to other biomarkers that are not highly correlated with a presence or absence of cancer.

In some embodiments, the importance of each biomarker is determined by using a method including one of random forest (RF), gradient boosting (GBM), extreme gradient boosting (XGB), or LASSO algorithms. For example, if using random forest algorithms, the random forest algorithm may provide, for each biomarker, 1) a mean decrease in model accuracy and/or 2) a mean decrease in a Gini coefficient which is a measure of how much each biomarker contributes to the homogeneity of nodes and leaves in the random forest. In one scenario, the importance of each biomarker is dependent on one or both of the mean decrease in model accuracy and mean decrease in Gini coefficient.

In various embodiments, the model training module 150 trains a predictive model to achieve certain performance metrics. Performance metrics include, but are not limited to, area under a receiver operating characteristic curve (AUC), accuracy, sensitivity, specificity, positive predictive value, true positive rate, true negative rate, false positive rate, false negative rate, negative predictive value, or false discovery rate. As used herein, accuracy refers to the ratio of the sum of true positives and true negatives divided by the sum of all positives and negatives. Sensitivity is used herein as the ratio of true positives divided by the sum of true positives and false negatives. Specificity is used herein as the ratio of true negatives divided by the sum of true negatives and false positives. Positive predictive value is used herein as the ratio of true positives divided by the sum of true positives and false positives. Negative predictive value is used herein as the ratio of true negatives divided by the sum of true negatives and false negatives. True positive rate, as used herein, refers to the rate of correct classification by the model of the cancer status in a subject as positive. True negative rate, as used herein, refers to the rate of correct classification by the model of the cancer status in a subject as negative. False positive rate, as used herein, refers to the rate of incorrect classification by the model of the cancer status in a subject as positive. False negative rate, as used herein, refers to the rate of incorrect classification by the model of the cancer status in a subject as negative. False discovery rate, as used herein, refers to the expected proportion of false discoveries among all discoveries.

In various embodiments, the model training module 150 trains a predictive model which achieves a particular AUC performance metric. In various embodiments, the predictive model achieves an AUC of at least 0.60, at least 0.61, at least 0.62, at least 0.63, at least 0.64, at least 0.65, at least 0.66, at least 0.67, at least 0.68, at least 0.69, at least 0.70, at least 0.71, at least 0.72, at least 0.73, at least 0.74, at least 0.75, at least 0.76, at least 0.77, at least 0.78, at least 0.79, at least 0.80, at least 0.81, at least 0.82, at least 0.83, at least 0.84, at least 0.85, at least 0.86, at least 0.87, at least 0.88, at least 0.89, at least 0.90, at least 0.91, at least 0.92, at least 0.93, at least 0.94, at least 0.95, at least 0.96, at least 0.97, at least 0.98, or at least 0.99. In various embodiments, the predictive model achieves an AUC of at least 0.60. In various embodiments, the predictive model achieves an AUC of at least 0.61. In various embodiments, the predictive model achieves an AUC of at least 0.62. In various embodiments, the predictive model achieves an AUC of at least 0.63. In various embodiments, the predictive model achieves an AUC of at least 0.64. In various embodiments, the predictive model achieves an AUC of at least 0.65. In various embodiments, the predictive model achieves an AUC of at least 0.66. In various embodiments, the predictive model achieves an AUC of at least 0.67. In various embodiments, the predictive model achieves an AUC of at least 0.68. In various embodiments, the predictive model achieves an AUC of at least 0.69. In various embodiments, the predictive model achieves an AUC of at least 0.70. In various embodiments, the predictive model achieves an AUC of at least 0.71. In various embodiments, the predictive model achieves an AUC of at least 0.72. In various embodiments, the predictive model achieves an AUC of at least 0.73. In various embodiments, the predictive model achieves an AUC of at least 0.74. In various embodiments, the predictive model achieves an AUC of at least 0.75. In various embodiments, the predictive model achieves an AUC of at least 0.76. In various embodiments, the predictive model achieves an AUC of at least 0.77. In various embodiments, the predictive model achieves an AUC of at least 0.78. In various embodiments, the predictive model achieves an AUC of at least 0.79. In various embodiments, the predictive model achieves an AUC of at least 0.80. In various embodiments, the predictive model achieves an AUC of at least 0.81. In various embodiments, the predictive model achieves an AUC of at least 0.82. In various embodiments, the predictive model achieves an AUC of at least 0.83. In various embodiments, the predictive model achieves an AUC of at least 0.84. In various embodiments, the predictive model achieves an AUC of at least 0.85. In various embodiments, the predictive model achieves an AUC of at least 0.86. In various embodiments, the predictive model achieves an AUC of at least 0.87. In various embodiments, the predictive model achieves an AUC of at least 0.88. In various embodiments, the predictive model achieves an AUC of at least 0.89. In various embodiments, the predictive model achieves an AUC of at least 0.90. In various embodiments, the predictive model achieves an AUC of at least 0.91. In various embodiments, the predictive model achieves an AUC of at least 0.92. In various embodiments, the predictive model achieves an AUC of at least 0.93. In various embodiments, the predictive model achieves an AUC of at least 0.94. In various embodiments, the predictive model achieves an AUC of at least 0.95. In various embodiments, the predictive model achieves an AUC of at least 0.96. In various embodiments, the predictive model achieves an AUC of at least 0.97. In various embodiments, the predictive model achieves an AUC of at least 0.98. In various embodiments, the predictive module achieves an AUC of at least 0.99.

In various embodiments, the model training module 150 trains a predictive model which achieves a particular accuracy performance metric. In various embodiments, the predictive model achieves an accuracy of at least 0.60, at least 0.61, at least 0.62, at least 0.63, at least 0.64, at least 0.65, at least 0.66, at least 0.67, at least 0.68, at least 0.69, at least 0.70, at least 0.71, at least 0.72, at least 0.73, at least 0.74, at least 0.75, at least 0.76, at least 0.77, at least 0.78, at least 0.79, at least 0.80, at least 0.81, at least 0.82, at least 0.83, at least 0.84, at least 0.85, at least 0.86, at least 0.87, at least 0.88, at least 0.89, at least 0.90, at least 0.91, at least 0.92, at least 0.93, at least 0.94, at least 0.95, at least 0.96, at least 0.97, at least 0.98, or at least 0.99. In various embodiments, the predictive model achieves an accuracy of at least 0.60. In various embodiments, the predictive model achieves an accuracy of at least 0.61. In various embodiments, the predictive model achieves an accuracy of at least 0.62. In various embodiments, the predictive model achieves an accuracy of at least 0.63. In various embodiments, the predictive model achieves an accuracy of at least 0.64. In various embodiments, the predictive model achieves an accuracy of at least 0.65. In various embodiments, the predictive model achieves an accuracy of at least 0.66. In various embodiments, the predictive model achieves an accuracy of at least 0.67. In various embodiments, the predictive model achieves an accuracy of at least 0.68. In various embodiments, the predictive model achieves an accuracy of at least 0.69. In various embodiments, the predictive model achieves an accuracy of at least 0.70. In various embodiments, the predictive model achieves an accuracy of at least 0.71. In various embodiments, the predictive model achieves an accuracy of at least 0.72. In various embodiments, the predictive model achieves an accuracy of at least 0.73. In various embodiments, the predictive model achieves an accuracy of at least 0.74. In various embodiments, the predictive model achieves an accuracy of at least 0.75. In various embodiments, the predictive model achieves an accuracy of at least 0.76. In various embodiments, the predictive model achieves an accuracy of at least 0.77. In various embodiments, the predictive model achieves an accuracy of at least 0.78. In various embodiments, the predictive model achieves an accuracy of at least 0.79. In various embodiments, the predictive model achieves an accuracy of at least 0.80. In various embodiments, the predictive model achieves an accuracy of at least 0.81. In various embodiments, the predictive model achieves an accuracy of at least 0.82. In various embodiments, the predictive model achieves an accuracy of at least 0.83. In various embodiments, the predictive model achieves an accuracy of at least 0.84. In various embodiments, the predictive model achieves an accuracy of at least 0.85. In various embodiments, the predictive model achieves an accuracy of at least 0.86. In various embodiments, the predictive model achieves an accuracy of at least 0.87. In various embodiments, the predictive model achieves an accuracy of at least 0.88. In various embodiments, the predictive model achieves an accuracy of at least 0.89. In various embodiments, the predictive model achieves an accuracy of at least 0.90. In various embodiments, the predictive model achieves an accuracy of at least 0.91. In various embodiments, the predictive model achieves an accuracy of at least 0.92. In various embodiments, the predictive model achieves an accuracy of at least 0.93. In various embodiments, the predictive model achieves an accuracy of at least 0.94. In various embodiments, the predictive model achieves an accuracy of at least 0.95. In various embodiments, the predictive model achieves an accuracy of at least 0.96. In various embodiments, the predictive model achieves an accuracy of at least 0.97. In various embodiments, the predictive model achieves an accuracy of at least 0.98. In various embodiments, the predictive module achieves an accuracy of at least 0.99.

In various embodiments, the model training module 150 trains a predictive model which achieves a true positive rate of at least 0.8 at a false positive rate of 0.25. In various embodiments, the model training module 150 trains a predictive model which achieves a true positive rate of at least 0.81, at least 0.82, at least 0.83, at least 0.84, at least 0.85, at least 0.86, at least 0.87, at least 0.88, at least 0.89, at least 0.90, at least 0.91, at least 0.92, at least 0.93, at least 0.94, at least 0.95, at least 0.96, at least 0.97, at least 0.98, at least 0.99, or 1.0 at a false positive rate of 0.25. In various embodiments, the model training module 150 trains a predictive model which achieves a true positive rate of at least 0.8 at a false positive rate of 0.2. In various embodiments, the model training module 150 trains a predictive model which achieves a true positive rate of at least 0.81, at least 0.82, at least 0.83, at least 0.84, at least 0.85, at least 0.86, at least 0.87, at least 0.88, at least 0.89, at least 0.90, at least 0.91, at least 0.92, at least 0.93, at least 0.94, at least 0.95, at least 0.96, at least 0.97, at least 0.98, at least 0.99, or 1.0 at a false positive rate of 0.2. In various embodiments, the model training module 150 trains a predictive model which achieves a true positive rate of at least 0.8 at a false positive rate of 0.1. In various embodiments, the model training module 150 trains a predictive model which achieves a true positive rate of at least 0.81, at least 0.82, at least 0.83, at least 0.84, at least 0.85, at least 0.86, at least 0.87, at least 0.88, at least 0.89, at least 0.90, at least 0.91, at least 0.92, at least 0.93, at least 0.94, at least 0.95, at least 0.96, at least 0.97, at least 0.98, at least 0.99, or 1.0 at a false positive rate of 0.1.

In various embodiments, the model training module 150 trains a predictive model which achieves a true positive rate of at least 10% to 100% at a false positive rate of 0% to 30%. In various embodiments, the model training module 150 trains a predictive model which achieves a true positive rate of at least 20% to 100% at a false positive rate of 0% to 20%. In various embodiments, the model training module 150 trains a predictive model which achieves a true positive rate of at least 20% to 100% at a false positive rate of 0% to 10%. In various embodiments, the model training module 150 trains a predictive model which achieves a true positive rate of at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% at a false positive rate of 0%, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, or 30%. In various embodiments, the model training module 150 trains a predictive model which achieves a true positive rate of at least 30% at a false positive rate of 10%.

### III.B. Deploying a Predictive Model

During the deployment phase, the model deployment module 160 (as shown in FIG. 1B) analyzes quantitative biomarker expression values from a test sample obtained from a subject of interest by applying a trained predictive model. Generally, the predictive model analyzes the biomarker expression value and outputs a prediction, such as a score informative for determining a presence or absence of cancer in the subject.

In various embodiments, the score represents a combination of the changed expressions of the plurality of biomarkers in the test sample obtained from the subject (e.g., changed expression in comparison to one or more healthy controls). In various embodiments, if all or a majority of the expression values of biomarkers are trending in a particular direction (e.g., upregulation or downregulation in comparison to healthy), then the subject can be deemed as having a presence of cancer. Alternatively, if all or a majority of the expression values of biomarkers are not trending in a particular direction (e.g., not upregulated or downregulated in comparison to healthy), then the subject can be deemed as having an absence of cancer. Table 2 and Table 3 below shows exemplary biomarkers and the median expression values of the biomarkers in cancer samples and in non-cancer samples. For example, referring to the second and third biomarkers in Table 2 (e.g., Complement C3 and Oxidized low-density lipoprotein receptor 1), both of the biomarkers have a higher median expression value in cancer samples in comparison to non-cancer samples. Therefore, if a subject presents with a test sample in which the expression levels of Complement C3 and Oxidized low-density lipoprotein receptor 1 are both upregulated in comparison to a healthy control, the subject can be classified as having a presence of cancer. This methodology can be similarly applied to any of the other biomarkers, or combinations of the other biomarkers, shown in Table 2, Table 3, Table 4, and/or Table 5.

In various embodiments, the score represents an aggregate score of the dysregulated expression of the plurality of biomarkers in the panel. In such embodiments, it is not necessary to know how the expression level of any individual biomarker has changed (relative to healthy control(s)) to classify the subject as having a presence or absence of cancer. Rather, it is the aggregate combination of how the biomarkers of the panel have changed relative to healthy control(s) that are determinative of whether the subject has a presence or absence of cancer. In particular embodiments, the predictive model is constructed such that one or more parameters (e.g., coefficients) are assigned to each biomarker. Here, a parameter may represent the importance of the particular biomarker associated with the parameter in determining the cancer prediction. Thus, the predictive model may more heavily consider the expression level of certain biomarkers (e.g., those associated with parameters of higher values) in comparison to other biomarkers (e.g., those associated with parameters of lower values) when determining the cancer prediction.

In various embodiments, predicting presence of absence of cancer in the subject involves comparing the predicted score outputted by the predictive model to one or more reference scores. As used herein, “reference scores” refer to previously determined scores, such as a “healthy reference score” corresponding to one or more healthy patients or a “cancer reference score” corresponding to one or more cancerous patients. For example, a healthy reference score may correspond to healthy patients, a patient's own baseline at a prior timepoint when the patient did not exhibit cancer activity (e.g., longitudinal analysis), patients clinically diagnosed with cancer but not exhibiting cancer activity (e.g., cancer remission), or a healthy reference threshold score (e.g., a cutoff). As another example, a “cancer reference score” may correspond to patients previously diagnosed with cancer, patients exhibiting cancer activity, or a cancer reference threshold score (e.g., a cutoff). In various embodiments, the threshold score can be derived from a cancer case/non-cancer control ROC curve analysis. The ROC curve can be derived using a logistic regression probability, or any other predictive method that can calculate a score that may be used for classification (e.g., for instance, a neural network).

In various embodiments, a reference score can be a threshold cutoff score with a value between 0 and 1. In various embodiments, the threshold cutoff score is any of 0.001, 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, or 0.95. In particular embodiments, the threshold cutoff score is between 0.5 and 1.0. In particular embodiments, the threshold cutoff score is between 0.6 and 0.8. In particular embodiments, the threshold cutoff score is 0.7.

In various embodiments, predicting presence of absence of cancer in the subject involves determining whether the predicted score outputted by the predictive model is above or below the threshold cutoff score. In particular embodiments, if the predicted score is above the threshold cutoff score, the subject is determined to have a presence of cancer. If the predicted score is below the threshold cutoff score, the subject is determined to have an absence of cancer. In some embodiments, if the predicted score is above the threshold cutoff score, the subject is determined to have an absence of cancer. If the predicted score is below the threshold cutoff score, the subject is determined to have a presence of cancer.

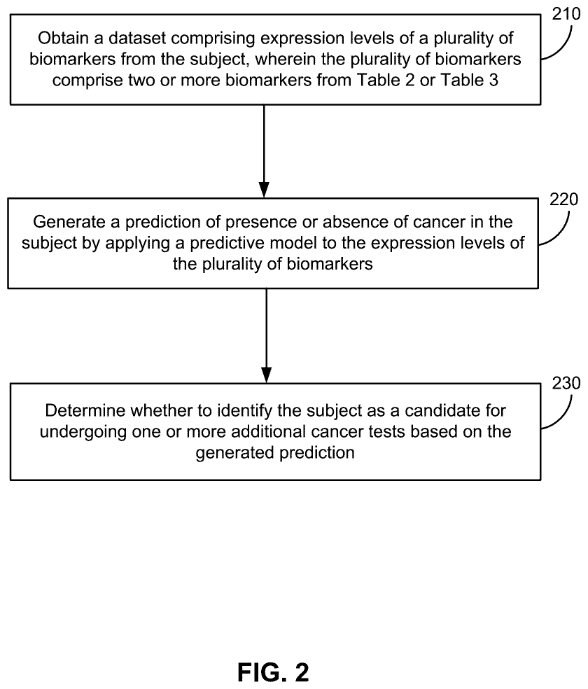

FIG. 2 depicts a flow diagram for generating a cancer prediction for a subject, in accordance with an embodiment. In particular embodiments, the cancer prediction is a presence or absence of cancer in the subject, such as presence of absence of early stage cancer in the subject.

Step 210 involves obtaining a dataset comprising expression levels of a plurality of biomarkers from the subject. In various embodiments, the plurality of biomarkers comprise two or more biomarkers selected from the biomarkers detailed in Table 2 or Table 3.

Step 220 involves generating a cancer prediction (e.g., a prediction of presence or absence of cancer) for the subject by applying a predictive model to the expression levels of the plurality of biomarkers. The predictive model outputs a prediction, such as a score informative for determining a presence or absence of cancer in the subject. In various embodiments, the score outputted by the predictive model is compared to a threshold score to classify the subject as having a presence or absence of cancer.

Step 230 involves determining whether to identify the subject as a candidate for undergoing one or more additional tests based on the generated cancer prediction. In various embodiments, responsive to determining that the subject likely has a presence of cancer, step 230 can involve performing a performing a second analysis to predict presence or absence of the early stage cancer or non-early stage cancer in a subject. In such embodiments, the predictive model at step 220 may be a high sensitivity predictive model that enables the rapid screening out of subjects who do not have cancer with high accuracy. Step 230 may involve a second analysis that further distinguishes the remaining subjects as having a presence or absence of cancer. Here, the second analysis can achieve a higher specificity in comparison to a specificity of the predictive model, thereby enabling the identification of the true positives (e.g., those subjects truly having a presence of cancer). In various embodiments, the one or more additional tests includes one or more of further blood molecular testing, a computerized tomography (CT) scan, a positron emission tomography (PET) scan, or a tissue biopsy. In various embodiments, the one or more additional tests may be sequentially performed depending on the results of the prior test. For example, responsive to determining that the subject likely has a presence of cancer, a CT scan or a PET scan can be performed. If the CT scan or PET scan further confirms a signal indicative of presence of cancer (e.g., presence of a mass in the scan), then a tissue biopsy can be subsequently performed.

### IV. Biomarker Panel and Biomarkers

In various embodiments, generating a cancer prediction involves implementing a univariate biomarker panel. Therefore, the univariate biomarker panel includes one biomarker. In various embodiments, an example univariate biomarker panel can include any one of the biomarkers detailed in Table 2. In other embodiments, generating a cancer prediction involves implementing a multivariate biomarker panel. In such embodiments, the multivariate biomarker panel includes more than one biomarker.

In various embodiments, the multivariate biomarker panel includes two biomarkers. In various embodiments, an example multivariate biomarker panel can include any of the biomarker combinations detailed in Table 4 or Table 5. In various embodiments, an example multivariate biomarker panel can include any of the biomarker combinations detailed in Table 4. In various embodiments, an example multivariate biomarker panel can include any of the biomarker combinations detailed in Table 5. In various embodiments, the multivariate biomarker panel includes 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, or 400 biomarkers. In various embodiments, the multivariate biomarker panel includes at least 2 biomarkers, at least 5 biomarkers, at least 8 biomarkers, at least 10 biomarkers, at least 12 biomarkers, at least 15 biomarkers, at least 16 biomarkers, at least 18 biomarkers, at least 20 biomarkers, at least 21 biomarkers, at least 22 biomarkers, at least 23 biomarkers, at least 24 biomarkers, at least 25 biomarkers, at least 28 biomarkers, at least 30 biomarkers, at least 35 biomarkers, at least 40 biomarkers, at least 45 biomarkers, at least 50 biomarkers, at least 60 biomarkers, at least 70 biomarkers, at least 80 biomarkers, at least 90 biomarkers, at least 100 biomarkers, at least 110 biomarkers, at least 120 biomarkers, at least 130 biomarkers, at least 140 biomarkers, at least 150 biomarkers, at least 175 biomarkers, at least 200 biomarkers, at least 250 biomarkers, at least 300 biomarkers, at least 350 biomarkers, or at least 400 biomarkers.

Example biomarkers included in a biomarker panel can include one or more of, two or more of, three or more of, four or more of, five or more of, six or more of, seven or more of, eight or more of, nine or more of, ten or more of, eleven or more of, twelve or more of, thirteen or more of, fourteen or more of, fifteen or more of, sixteen or more of, seventeen or more of, eighteen or more of, nineteen or more of, twenty or more of, twenty or more of, twenty two or more of, twenty three or more of, twenty four or more of, or twenty five or more of Neurotrophin-3, Complement C3, Oxidized low-density lipoprotein receptor 1, Matrix metalloproteinase-9, Macrophage colony-stimulating factor 1, Oncostatin-M, Tumor necrosis factor receptor superfamily member 1A, WAP four-disulfide core domain protein 2, C-type lectin domain family 5 member A, S-methylmethionine-homocysteine S-methyltransferase BHMT2, Urokinase plasminogen activator surface receptor, Protransforming growth factor alpha, Zinc finger protein GLI2, Neutrophil collagenase, Tumor necrosis factor receptor superfamily member 3, Interleukin-8, Monocyte differentiation antigen CD14, Protein shisa-5, CD59 glycoprotein, Neural proliferation differentiation and control protein 1, C-X-C motif chemokine 9, C-C motif chemokine 23, Collagen alpha-1(IV) chain, Placenta growth factor, Growth/differentiation factor 15, Collagen alpha-1(XVIII) chain, Natural cytotoxicity triggering receptor 3 ligand 1, Stromal cell-derived factor 1, Hepatitis A virus cellular receptor 2, Huntingtin-interacting protein 1-related protein, Retinoid-binding protein 7, Kunitz-type protease inhibitor 1, Latent-transforming growth factor beta-binding protein 2, Calbindin, RNA binding protein fox-1 homolog 3, Occludin, GDNF family receptor alpha-1, Follistatin-related protein 3, Ephrin-A1, Basigin, Leucine-rich alpha-2-glycoprotein, Tumor necrosis factor receptor superfamily member 19L, Fibrinogen alpha chain, Inter-alpha-trypsin inhibitor heavy chain H3, Metalloproteinase inhibitor 1, Tumor necrosis factor receptor superfamily member 1B, Carcinoembryonic antigen-related cell adhesion molecule 8, MAM domain-containing protein 2, Interleukin-6, Folate receptor alpha, Carcinoembryonic antigen-related cell adhesion molecule 5, Osteopontin, Macrophage-capping protein, Galectin-9, NPC intracellular cholesterol transporter 2, Gamma-interferon-inducible lysosomal thiol reductase, Elastin, Macrophage metalloelastase, V-set and immunoglobulin domain-containing protein 4, Nectin-2, Mitotic spindle assembly checkpoint protein MAD1, Tumor necrosis factor receptor superfamily member 27, Tumor necrosis factor receptor superfamily member 10B, Survival of motor neuron-related-splicing factor 30, Prostasin, C-X-C motif chemokine 17, Receptor-type tyrosine-protein phosphatase F, Tumor necrosis factor receptor superfamily member 10A, Cystatin-B, Triggering receptor expressed on myeloid cells 2, Syndecan-1, Desmocollin-2, Nucleoside diphosphate kinase A, Lamin-B2, Cytoskeleton-associated protein 4, Ephrin type-B receptor 4, Layilin, Delta-like protein 1, Bone marrow proteoglycan, Seizure 6-like protein 2, Collectin-12, UL16-binding protein 2, Beta-1,4-galactosyltransferase 1, Hydroxyacylglutathione hydrolase, mitochondrial, Neutrophil gelatinase-associated lipocalin, All-trans retinoic acid-induced differentiation factor, Interleukin-1 receptor antagonist protein, Transcriptional coactivator YAP1, Tumor necrosis factor ligand superfamily member 13, Cystatin-C, Tumor necrosis factor receptor superfamily member 4, C-C motif chemokine 18, DNA-directed RNA polymerases I, II, and III subunit RPABC2, Ephrin type-A receptor 2, Signal-regulatory protein beta-1, Ganglioside GM2 activator, U2 small nuclear ribonucleoprotein B″, Inter-alpha-trypsin inhibitor heavy chain H4, Fibulin-2, Tumor necrosis factor receptor superfamily member 9, Cadherin-2, Interleukin-18-binding protein, Spliceosome-associated protein CWC15 homolog, Ephrin-A4, Glial fibrillary acidic protein, A disintegrin and metalloproteinase with thrombospondin motifs 16, Secretogranin-1, Amphiregulin, C-C motif chemokine 14, Carcinoembryonic antigen-related cell adhesion molecule 6, Ribonuclease pancreatic, Serine protease inhibitor Kazal-type 1, CD302 antigen, Kallikrein-7, Neuropilin-2, Integrin beta-like protein 1, Myeloblastin, Agrin, Regulator of chromosome condensation, Thrombospondin-2, Protein disulfide isomerase CRELD1, EGF-containing fibulin-like extracellular matrix protein 1, Lysosome membrane protein 2, Complement component C9, Coiled-coil-helix-coiled-coil-helix domain-containing protein 10, mitochondrial, EF-hand domain-containing protein D1, Fibrinogen-like protein 1, Interleukin-10 receptor subunit beta, Kallikrein-4, Septin-8, Trefoil factor 3, Cytokine receptor-like factor 1, Collagen alpha-3(VI) chain, Oxygen-dependent coproporphyrinogen-III oxidase, mitochondrial, Disintegrin and metalloproteinase domain-containing protein 8, C4b-binding protein beta chain, C-X-C motif chemokine 16, Leukocyte-associated immunoglobulin-like receptor 1, Scavenger receptor class F member 2, Serpin B8, Interleukin-4 receptor subunit alpha, CD276 antigen, Cadherin-23, Angiopoietin-2, Serine/threonine-protein kinase receptor R3, Cathepsin L2, Polypeptide N-acetylgalactosaminyltransferase 5, E3 SUMO-protein ligase RanBP2, Vasorin, von Willebrand factor A domain-containing protein 1, Ribonuclease K6, Apolipoprotein A-II, Intercellular adhesion molecule 1, Interleukin-2 receptor subunit alpha, Zinc finger and BTB domain-containing protein 17, Oncostatin-M-specific receptor subunit beta, GrpE protein homolog 1, mitochondrial, Insulin-like growth factor-binding protein 4, Vascular cell adhesion protein 1, Azurocidin, Cathepsin D, Ribonuclease T2, Complement component Cq receptor, Sushi domain-containing protein 5, SLAM family member 8, C-C motif chemokine 26, Insulin-like growth factor-binding protein 2, E3 ubiquitin-protein ligase RNF149, Tyrosine-protein kinase Mer, Protein S100-A11, Sushi, nidogen and EGF-like domain-containing protein 1, Carcinoembryonic antigen-related cell adhesion molecule 21, E3 ubiquitin-protein ligase UHRF2, Beta-Ala-His dipeptidase, Nectin-4, Polymeric immunoglobulin receptor, Sprouty-related, EVH1 domain-containing protein 2, Vasoactive intestinal polypeptide receptor 1, Galactoside 3(4)-L-fucosyltransferase and Alpha-(1,3)-fucosyltransferase 5, Protein S100-A12, Tumor necrosis factor receptor superfamily member 111B, Interferon gamma receptor 1, Nucleophosmin, Actin, aortic smooth muscle, Keratin, type I cytoskeletal 19, Sialic acid-binding Ig-like lectin 5, Lysosome-associated membrane glycoprotein 3, CD166 antigen, HLA class II histocompatibility antigen gamma chain, Proline-rich transmembrane protein 3, Integrin alpha-5, Trans-Golgi network integral membrane protein 2, CUB domain-containing protein 1, Creatine kinase B-type, Protein S100-P, Serpin A11, Paired immunoglobulin-like type 2 receptor alpha, Annexin A1, Band 3 anion transport protein, Neutrophil cytosol factor 2, Pentraxin-related protein PTX3, Lymphocyte-specific protein 1, CMRF35-like molecule 8, C-type lectin domain family 7 member A, Lysophosphatidylcholine acyltransferase 2, Neuropilin-1, MICOS complex subunit MIC25, Alpha-1-antichymotrypsin, Tumor necrosis factor receptor superfamily member 21, Dipeptidyl peptidase 1, Leukocyte immunoglobulin-like receptor subfamily B member 4, Nibrin, Complement decay-accelerating factor, Beta-2-microglobulin, Arginase-1, Tumor necrosis factor receptor superfamily member 16, 26S proteasome non-ATPase regulatory subunit 1, Signal recognition particle 14 kDa protein, Integrin beta-6, AMP deaminase 3, CMRF35-like molecule 2, Polycystin-2, Stanniocalcin-2, GTP cyclohydrolase 1 feedback regulatory protein, Peptidoglycan recognition protein 1, Paired immunoglobulin-like type 2 receptor beta, Cadherin-3, Nicotinamide riboside kinase 2, Mothers against decapentaplegic homolog 1, Discoidin, CUB and LCCL domain-containing protein 2, Cysteine-rich motor neuron 1 protein, Heparan-sulfate 6-O-sulfotransferase 2, Tumor necrosis factor receptor superfamily member 8, 1,25-dihydroxyvitamin D(3) 24-hydroxylase, mitochondrial, BH3-interacting domain death agonist, Glutaredoxin-1, Tumor necrosis factor receptor superfamily member 14, Dipeptidase 2, Coagulation factor IX, Prostaglandin-H2 D-isomerase, Complement C2, Erythroid membrane-associated protein, Insulin-like growth factor-binding protein-like 1, Cystatin-SN, Elongin-A, Mucin-13, Interleukin-1 receptor type 1, Protein S100-A3, Phosphoinositide-3-kinase-interacting protein 1, Vascular non-inflammatory molecule 2, Thiopurine S-methyltransferase, Angiopoietin-related protein 3, Asialoglycoprotein receptor 1, Bone morphogenetic protein 4, C-type lectin domain family 4 member D, Basement membrane-specific heparan sulfate proteoglycan core protein, C-C motif chemokine 3, CMRF35-like molecule 1, Collagen alpha-1(XXVIII) chain, C-X-C motif chemokine 10, Glutaminyl-peptide cyclotransferase, TGF-beta receptor type-2, Collagen alpha-1(XXIV) chain, Cadherin-6, CMRF35-like molecule 6, Follistatin, Myosin-binding protein C, fast-type, BTB/POZ domain-containing protein KCTD5, Granulocyte colony-stimulating factor, Interleukin-27, Zinc transporter ZIP14, Interleukin-7, Carbonic anhydrase 1, Torsin-1A-interacting protein 1, Chitinase-3-like protein 1, Protein DGCR6, Tenascin, C-type lectin domain family 4 member G, Colipase, Beta-enolase, Epsin-1, Receptor-type tyrosine-protein phosphatase N2, Pro-adrenomedullin, Leukotriene A-4 hydrolase, Treacle protein, T-cell immunoglobulin and mucin domain-containing protein 4, C-C motif chemokine 28, Kallikrein-11, Kallikrein-6, Lymphatic vessel endothelial hyaluronic acid receptor 1, Protein-glutamine gamma-glutamyltransferase 2, Secreted frizzled-related protein 3, Disintegrin and metalloproteinase domain-containing protein 9, Alpha-hemoglobin-stabilizing protein, C-C motif chemokine 2, Egl nine homolog 1, Macrophage mannose receptor 1, Microtubule-associated tumor suppressor 1, 40S ribosomal protein S10, Tumor-associated calcium signal transducer 2, Serum amyloid A-4 protein, SLIT and NTRK-like protein 6, Citron Rho-interacting kinase, Tumor necrosis factor receptor superfamily member 19, MICOS complex subunit MIC60, Alpha-1-acid glycoprotein 1, Collagen triple helix repeat-containing protein 1, Dyslexia-associated protein KIAA0319, Butyrophilin subfamily 2 member A1, Alpha-1B-glycoprotein, Draxin, Fibroblast growth factor 6, Semaphorin-3F, Stanniocalcin-1, Basal cell adhesion molecule, Chromatin complexes subunit BAP18, C-C motif chemokine 16, Dickkopf-related protein 3, Podocalyxin-like protein 2, von Willebrand factor, Pseudokinase FAM20A, Density-regulated protein, Insulin-like growth factor-binding protein 7, Growth/differentiation factor 8, Enolase-phosphatase E1, Tetraspanin-1, EF-hand calcium-binding domain-containing protein 14, Protein AMBP, Complement C1r subcomponent-like protein, Interleukin-5, Tumor necrosis factor ligand superfamily member 14, Hepatitis A virus cellular receptor 1, Tumor necrosis factor receptor superfamily member 12A, Collagen alpha-1(III) chain, G-patch domain and KOW motifs-containing protein, MANSC domain-containing protein 1, Protein sel-1 homolog 1, Periostin, PDZ domain-containing protein GIPC2, Dual adapter for phosphotyrosine and 3-phosphotyrosine and 3-phosphoinositide, Decorin, Tumor necrosis factor receptor superfamily member 6, Putative oxidoreductase GLYR1, Lipocalin-15, Neurofilament light polypeptide, Ubiquitin carboxyl-terminal hydrolase 28, Chondroadherin, Corticoliberin, Phenazine biosynthesis-like domain-containing protein, Proliferating cell nuclear antigen, Granulocyte-macrophage colony-stimulating factor, Lymphokine-activated killer T-cell-originated protein kinase, Brain-derived neurotrophic factor, Inactive tyrosine-protein kinase transmembrane receptor ROR1, Ficolin-1, Angiopoietin-related protein 4, Protein ZNRD2, Fractalkine, Myosin-7B, NAD kinase, Ras-related protein Rab-44, Tumor necrosis factor receptor superfamily member 11A, Tumor necrosis factor receptor superfamily member 6B, CXADR-like membrane protein, Histone deacetylase 8, Immunoglobulin superfamily member 8, Paralemmin-2, Reversion-inducing cysteine-rich protein with Kazal motifs, C-type lectin domain family 14 member A, Peptidyl-prolyl cis-trans isomerase FKBP1B, Interleukin-13 receptor subunit alpha-1, Protein Wnt-9a, Phospholipid transfer protein C2CD2L, Coiled-coil domain-containing protein 80, Phospholipase A2, membrane associated, U4/U6.U5 tri-snRNP-associated protein 1, Kin of IRRE-like protein 2, C-C motif chemokine 4, Interleukin-18 receptor 1, Neogenin, Leucine-rich repeat transmembrane protein FLRT2, Tissue factor pathway inhibitor 2, Delta(14)-sterol reductase LBR, Immunoglobulin superfamily containing leucine-rich repeat protein 2, Leukocyte cell-derived chemotaxin-2, Pancreatic prohormone, Alpha-1-antitrypsin, Brorin, Protein FAM3C, Porphobilinogen deaminase, Lamin-B1, Brain-specific serine protease 4, Calcitonin gene-related peptide 2, C-C motif chemokine 7, Cathepsin L1, Folate receptor beta, Prosaposin, Semaphorin-7A, N-acetylgalactosaminyltransferase 7, Cytosolic 5′-nucleotidase 1A, Fibroblast growth factor receptor 4, Flavin reductase (NADPH), BPI fold-containing family B member 2, CCN family member 3, G-protein coupled receptor family C group 5 member C, Phosphatidylinositol 4,5-bisphosphate 5-phosphatase A, Fibroblast growth factor receptor 2, CD83 antigen, Scrapie-responsive protein 1, Aldehyde dehydrogenase, dimeric NADP-preferring, Cytokine-like protein 1, Osteoclast-associated immunoglobulin-like receptor, Pleckstrin homology-like domain family B member 1, Tumor necrosis factor ligand superfamily member 11, Appetite-regulating hormone, Ribonucleoside-diphosphate reductase subunit M2, Adhesion G-protein coupled receptor G1, Tyrosine-protein kinase receptor UFO, Carbonic anhydrase 14, Complement factor H, Interleukin-6 receptor subunit alpha, Galectin-3, Spondin-2, Calcyphosin, dCTP pyrophosphatase 1, Macrophage scavenger receptor types I and II, Retinoic acid receptor responder protein 2, Sodium channel protein type 3 subunit alpha, VPS10 domain-containing receptor SorCS2, Secretogranin-2, Beta-crystallin B2, DnaJ homolog subfamily A member 4, Leukocyte immunoglobulin-like receptor subfamily A member 5, Renin, Cochlin, C-type lectin domain family 11 member A, Corticotropin-releasing factor-binding protein, Phenylalanine-tRNA ligase alpha subunit, Nephrin, Melanoma antigen preferentially expressed in tumors, Peroxiredoxin-2, C-X-C motif chemokine 13, Asialoglycoprotein receptor 2, Protein BRICK1, Retinoid-inducible serine carboxypeptidase, Neuroendocrine secretory protein 55, Bcl-2-like protein 15, Uncharacterized protein C9orf40, Immunoglobulin superfamily member 2, Cathepsin Z, Endothelial cell-specific molecule 1, Cadherin-17, Complement C5, Serum paraoxonase/arylesterase 1, Olfactomedin-4, Opticin, Paralemmin-1, Inactive pancreatic lipase-related protein 1, Paxillin, Ras/Rap GTPase-activating protein SynGAP, Beta-microseminoprotein, Hephaestin, Neugrin, Cell growth regulator with EF hand domain protein 1, Leukocyte immunoglobulin-like receptor subfamily B member 2, Neuritin, Branched-chain-amino-acid aminotransferase, mitochondrial, Heterogeneous nuclear ribonucleoprotein U-like protein 1, Early placenta insulin-like peptide, Myeloperoxidase, and Periplakin. Additional details of example biomarkers are detailed below in Table 2 and Table 3. In particular embodiments, biomarkers included in a biomarker panel can include two or more of the biomarkers detailed in Table 2 or Table 3. In particular embodiments, biomarkers included in a biomarker panel can include two or more of the biomarkers detailed in Table 4 or Table 5. In particular embodiments, biomarkers included in a biomarker panel can include the sets of biomarkers detailed in Table 4 or Table 5. In particular embodiments, biomarkers included in a biomarker panel can include any combination of the sets of biomarkers detailed in Table 4 or Table 5.

In various embodiments, the biomarkers of a biomarker panel comprise LTBR and at least a second biomarker. In various embodiments, the second biomarker is either LCN15 or OLRT. In various embodiments, the biomarkers of a biomarker panel comprise LTBR, LCN15, and OLRT.

In various embodiments, the biomarkers of a biomarker panel comprise LTBP2 and at least a second biomarker. In various embodiments, the biomarkers of a biomarker panel comprise TGFA and at least a second biomarker. In various embodiments, the biomarkers of a biomarker panel comprise two or more of GDF15, LAMP3, and OSM. In various embodiments, the biomarkers of a biomarker panel comprise each of GDF15, LAMP3, and OSM.

In various embodiments, the biomarkers of a biomarker panel comprise two or more of BID, COL4A1, NTF3, PPY, and PRSS22. In various embodiments, the biomarkers of a biomarker panel comprise three or more of BID, COL4A1, NTF3, PPY, and PRSS22. In various embodiments, the biomarkers of a biomarker panel comprise four or more of BID, COL4A1, NTF3, PPY, and PRSS22. In various embodiments, the biomarkers of a biomarker panel comprise each of BID, COL4A1, NTF3, PPY, and PRSS22.

In various embodiments, the biomarkers of a biomarker panel comprise two or more of CLPS, LTBR, and MMP9. In various embodiments, the biomarkers of a biomarker panel comprise each of CLPS, LTBR, and MMP9.

In various embodiments, the biomarkers of a biomarker panel comprise two or more of HEPH, ITGBL1, OSM, and SCARF2. In various embodiments, the biomarkers of a biomarker panel comprise three or more of HEPH, ITGBL1, OSM, and SCARF2. In various embodiments, the biomarkers of a biomarker panel comprise each of HEPH, ITGBL1, OSM, and SCARF2.

In various embodiments, the biomarkers of a biomarker panel comprise ITGBL1 and MMP9. In various embodiments, the biomarkers of a biomarker panel comprise two or more of COL4A1, FGFR4, NTF3, and PPY. In various embodiments, the biomarkers of a biomarker panel comprise three or more of COL4A1, FGFR4, NTF3, and PPY. In various embodiments, the biomarkers of a biomarker panel comprise each of COL4A1, FGFR4, NTF3, and PPY.

In various embodiments, the biomarkers of a biomarker panel comprise two or more biomarkers selected from TGFA, S100A12, OSM, TFPI2, LSP1, MDK, CXCL9, CLEC4D, IL6, ALPP, HGF, VWA1, CEACAM5, MMP12, KRT19, CASP8, WFDC2, and PLAUR. In various embodiments, the biomarkers of a biomarker panel comprise two or more biomarkers selected from IL6, TGFA, S100A12, OSM, TFPI2, LSP1, MDK, CXCL9, CLEC4D, HGF, VWA1, CEACAM5, MMP12, KRT19, CASP8, WFDC2, and PLAUR. In various embodiments, the biomarkers of a biomarker panel comprise two or more biomarkers selected from TGFA, S100A12, OSM, TFPI2, LSP1, MDK, CXCL9, CLEC4D, HGF, VWA1, CEACAM5, MMP12, KRT19, CASP8, WFDC2, and PLAUR. In various embodiments, the biomarkers of a biomarker panel comprise IL6. In various embodiments, the biomarkers of a biomarker panel comprise TGFA. In various embodiments, the biomarkers of a biomarker panel comprise S100A12. In various embodiments, the biomarkers of a biomarker panel comprise OSM. In various embodiments, the biomarkers of a biomarker panel comprise TFPI2. In various embodiments, the biomarkers of a biomarker panel comprise LSP1. In various embodiments, the biomarkers of a biomarker panel comprise MDK. In various embodiments, the biomarkers of a biomarker panel comprise CXCL9. In various embodiments, the biomarkers of a biomarker panel comprise CLEC4D. In various embodiments, the biomarkers of a biomarker panel comprise HGF. In various embodiments, the biomarkers of a biomarker panel comprise VWA1. In various embodiments, the biomarkers of a biomarker panel comprise CEACAM5. In various embodiments, the biomarkers of a biomarker panel comprise MMP12. In various embodiments, the biomarkers of a biomarker panel comprise KRT19. In various embodiments, the biomarkers of a biomarker panel comprise CASP8. In various embodiments, the biomarkers of a biomarker panel comprise WFDC2. In various embodiments, the biomarkers of a biomarker panel comprise PLAUR. In various embodiments, the biomarkers of a biomarker panel comprise ALPP.

In various embodiments, the biomarkers of a biomarker panel comprise IL6 and at least one more biomarker selected from TGFA, S100A12, OSM, TFPI2, LSP1, MDK, CXCL9, CLEC4D, HGF, VWA1, CEACAM5, MMP12, KRT19, CASP8, WFDC2, ALPP, and PLAUR. In various embodiments, the biomarkers of a biomarker panel comprise IL6 and at least one more biomarker selected from TGFA, S100A12, OSM, TFPI2, LSP1, MDK, CXCL9, CLEC4D, HGF, VWA1, CEACAM5, MMP12, KRT19, CASP8, WFDC2, ALPP, and PLAUR. In various embodiments, the biomarkers of a biomarker panel comprise TGFA and at least one more biomarker selected from IL6, S100A12, OSM, TFPI2, LSP1, MDK, CXCL9, CLEC4D, HGF, VWA1, CEACAM5, MMP12, KRT19, CASP8, WFDC2, ALPP, and PLAUR. In various embodiments, the biomarkers of a biomarker panel comprise S100A12 and at least one more biomarker selected from IL6, TGFA, OSM, TFPI2, LSP1, MDK, CXCL9, CLEC4D, HGF, VWA1, CEACAM5, MMP12, KRT19, CASP8, WFDC2, ALPP, and PLAUR. In various embodiments, the biomarkers of a biomarker panel comprise OSM and at least one more biomarker selected from IL6, TGFA, S100A12, TFPI2, LSP1, MDK, CXCL9, CLEC4D, HGF, VWA1, CEACAM5, MMP12, KRT19, CASP8, WFDC2, ALPP, and PLAUR. In various embodiments, the biomarkers of a biomarker panel comprise TFPI2 and at least one more biomarker selected from IL6, TGFA, S100A12, OSM, LSP1, MDK, CXCL9, CLEC4D, HGF, VWA1, CEACAM5, MMP12, KRT19, CASP8, WFDC2, ALPP, and PLAUR. In various embodiments, the biomarkers of a biomarker panel comprise LSP1 and at least one more biomarker selected from IL6, TGFA, S100A12, OSM, TFPI2, MDK, CXCL9, CLEC4D, HGF, VWA1, CEACAM5, MMP12, KRT19, CASP8, WFDC2, ALPP, and PLAUR. In various embodiments, the biomarkers of a biomarker panel comprise MDK and at least one more biomarker selected from IL6, TGFA, S100A12, OSM, TFPI2, LSP1, CXCL9, CLEC4D, HGF, VWA1, CEACAM5, MMP12, KRT19, CASP8, WFDC2, ALPP, and PLAUR. In various embodiments, the biomarkers of a biomarker panel comprise CXCL9 and at least one more biomarker selected from IL6, TGFA, S100A12, OSM, TFPI2, LSP1, MDK, CLEC4D, HGF, VWA1, CEACAM5, MMP12, KRT19, CASP8, WFDC2, ALPP, and PLAUR. In various embodiments, the biomarkers of a biomarker panel comprise CLEC4D and at least one more biomarker selected from IL6, TGFA, S100A12, OSM, TFPI2, LSP1, MDK, CXCL9, HGF, VWA1, CEACAM5, MMP12, KRT19, CASP8, WFDC2, ALPP, and PLAUR. In various embodiments, the biomarkers of a biomarker panel comprise HGF and at least one more biomarker selected from IL6, TGFA, S100A12, OSM, TFPI2, LSP1, MDK, CXCL9, CLEC4D, VWA1, CEACAM5, MMP12, KRT19, CASP8, WFDC2, ALPP, and PLAUR. In various embodiments, the biomarkers of a biomarker panel comprise VWA1 and at least one more biomarker selected from IL6, TGFA, S100A12, OSM, TFPI2, LSP1, MDK, CXCL9, CLEC4D, HGF, CEACAM5, MMP12, KRT19, CASP8, WFDC2, ALPP, and PLAUR. In various embodiments, the biomarkers of a biomarker panel comprise CEACAM5 and at least one more biomarker selected from IL6, TGFA, S100A12, OSM, TFPI2, LSP1, MDK, CXCL9, CLEC4D, HGF, VWA1, MMP12, KRT19, CASP8, WFDC2, ALPP, and PLAUR. In various embodiments, the biomarkers of a biomarker panel comprise MMP12 and at least one more biomarker selected from IL6, TGFA, S100A12, OSM, TFPI2, LSP1, MDK, CXCL9, CLEC4D, HGF, VWA1, CEACAM5, KRT19, CASP8, WFDC2, ALPP, and PLAUR. In various embodiments, the biomarkers of a biomarker panel comprise KRT19 and at least one more biomarker selected from IL6, TGFA, S100A12, OSM, TFPI2, LSP1, MDK, CXCL9, CLEC4D, HGF, VWA1, CEACAM5, MMP12, CASP8, WFDC2, ALPP, and PLAUR. In various embodiments, the biomarkers of a biomarker panel comprise CASP8 and at least one more biomarker selected from IL6, TGFA, S100A12, OSM, TFPI2, LSP1, MDK, CXCL9, CLEC4D, HGF, VWA1, CEACAM5, MMP12, KRT19, WFDC2, ALPP, and PLAUR. In various embodiments, the biomarkers of a biomarker panel comprise WFDC2 and at least one more biomarker selected from IL6, TGFA, S100A12, OSM, TFPI2, LSP1, MDK, CXCL9, CLEC4D, HGF, VWA1, CEACAM5, MMP12, KRT19, CASP8, ALPP, and PLAUR. In various embodiments, the biomarkers of a biomarker panel comprise ALPP and at least one more biomarker selected from IL6, TGFA, S100A12, OSM, TFPI2, LSP1, MDK, CXCL9, CLEC4D, HGF, VWA1, CEACAM5, MMP12, KRT19, CASP8, WFDC2, and PLAUR. In various embodiments, the biomarkers of a biomarker panel comprise PLAUR and at least one more biomarker selected from IL6, TGFA, S100A12, OSM, TFPI2, LSP1, MDK, CXCL9, CLEC4D, HGF, VWA1, CEACAM5, MMP12, KRT19, CASP8, ALPP, and WFDC2.

In various embodiments, the biomarkers of a biomarker panel comprise IL6 and at least one more biomarker selected from TGFA, S100A12, OSM, TFPI2, LSP1, MDK, CXCL9, CLEC4D, HGF, VWA1, CEACAM5, MMP12, KRT19, CASP8, WFDC2, and PLAUR. In various embodiments, the biomarkers of a biomarker panel comprise IL6 and at least one more biomarker selected from TGFA, S100A12, OSM, TFPI2, LSP1, MDK, CXCL9, CLEC4D, HGF, VWA1, CEACAM5, MMP12, KRT19, CASP8, WFDC2, and PLAUR. In various embodiments, the biomarkers of a biomarker panel comprise TGFA and at least one more biomarker selected from IL6, S100A12, OSM, TFPI2, LSP1, MDK, CXCL9, CLEC4D, HGF, VWA1, CEACAM5, MMP12, KRT19, CASP8, WFDC2, and PLAUR. In various embodiments, the biomarkers of a biomarker panel comprise S100A12 and at least one more biomarker selected from IL6, TGFA, OSM, TFPI2, LSP1, MDK, CXCL9, CLEC4D, HGF, VWA1, CEACAM5, MMP12, KRT19, CASP8, WFDC2, and PLAUR. In various embodiments, the biomarkers of a biomarker panel comprise OSM and at least one more biomarker selected from IL6, TGFA, S100A12, TFPI2, LSP1, MDK, CXCL9, CLEC4D, HGF, VWA1, CEACAM5, MMP12, KRT19, CASP8, WFDC2, and PLAUR. In various embodiments, the biomarkers of a biomarker panel comprise TFPI2 and at least one more biomarker selected from IL6, TGFA, S100A12, OSM, LSP1, MDK, CXCL9, CLEC4D, HGF, VWA1, CEACAM5, MMP12, KRT19, CASP8, WFDC2, and PLAUR. In various embodiments, the biomarkers of a biomarker panel comprise LSP1 and at least one more biomarker selected from IL6, TGFA, S100A12, OSM, TFPI2, MDK, CXCL9, CLEC4D, HGF, VWA1, CEACAM5, MMP12, KRT19, CASP8, WFDC2, and PLAUR. In various embodiments, the biomarkers of a biomarker panel comprise MDK and at least one more biomarker selected from IL6, TGFA, S100A12, OSM, TFPI2, LSP1, CXCL9, CLEC4D, HGF, VWA1, CEACAM5, MMP12, KRT19, CASP8, WFDC2, and PLAUR. In various embodiments, the biomarkers of a biomarker panel comprise CXCL9 and at least one more biomarker selected from IL6, TGFA, S100A12, OSM, TFPI2, LSP1, MDK, CLEC4D, HGF, VWA1, CEACAM5, MMP12, KRT19, CASP8, WFDC2, and PLAUR. In various embodiments, the biomarkers of a biomarker panel comprise CLEC4D and at least one more biomarker selected from IL6, TGFA, S100A12, OSM, TFPI2, LSP1, MDK, CXCL9, HGF, VWA1, CEACAM5, MMP12, KRT19, CASP8, WFDC2, and PLAUR. In various embodiments, the biomarkers of a biomarker panel comprise HGF and at least one more biomarker selected from IL6, TGFA, S100A12, OSM, TFPI2, LSP1, MDK, CXCL9, CLEC4D, VWA1, CEACAM5, MMP12, KRT19, CASP8, WFDC2, and PLAUR. In various embodiments, the biomarkers of a biomarker panel comprise VWA1 and at least one more biomarker selected from IL6, TGFA, S100A12, OSM, TFPI2, LSP1, MDK, CXCL9, CLEC4D, HGF, CEACAM5, MMP12, KRT19, CASP8, WFDC2, and PLAUR. In various embodiments, the biomarkers of a biomarker panel comprise CEACAM5 and at least one more biomarker selected from IL6, TGFA, S100A12, OSM, TFPI2, LSP1, MDK, CXCL9, CLEC4D, HGF, VWA1, MMP12, KRT19, CASP8, WFDC2, and PLAUR. In various embodiments, the biomarkers of a biomarker panel comprise MMP12 and at least one more biomarker selected from IL6, TGFA, S100A12, OSM, TFPI2, LSP1, MDK, CXCL9, CLEC4D, HGF, VWA1, CEACAM5, KRT19, CASP8, WFDC2, and PLAUR. In various embodiments, the biomarkers of a biomarker panel comprise KRT19 and at least one more biomarker selected from IL6, TGFA, S100A12, OSM, TFPI2, LSP1, MDK, CXCL9, CLEC4D, HGF, VWA1, CEACAM5, MMP12, CASP8, WFDC2, and PLAUR. In various embodiments, the biomarkers of a biomarker panel comprise CASP8 and at least one more biomarker selected from IL6, TGFA, S100A12, OSM, TFPI2, LSP1, MDK, CXCL9, CLEC4D, HGF, VWA1, CEACAM5, MMP12, KRT19, WFDC2, and PLAUR. In various embodiments, the biomarkers of a biomarker panel comprise WFDC2 and at least one more biomarker selected from IL6, TGFA, S100A12, OSM, TFPI2, LSP1, MDK, CXCL9, CLEC4D, HGF, VWA1, CEACAM5, MMP12, KRT19, CASP8, and PLAUR. In various embodiments, the biomarkers of a biomarker panel comprise PLAUR and at least one more biomarker selected from IL6, TGFA, S100A12, OSM, TFPI2, LSP1, MDK, CXCL9, CLEC4D, HGF, VWA1, CEACAM5, MMP12, KRT19, CASP8, and WFDC2.

In various embodiments, the biomarkers of a biomarker panel comprise IL6 and at least one more biomarker selected from TGFA, S100A12, OSM, LSP1, MDK, CXCL9, HGF, CEACAM5, MMP12, KRT19, WFDC2, and PLAUR. In various embodiments, the biomarkers of a biomarker panel comprise IL6 and at least one more biomarker selected from TGFA, S100A12, OSM, LSP1, MDK, CXCL9, HGF, CEACAM5, MMP12, KRT19, WFDC2, and PLAUR. In various embodiments, the biomarkers of a biomarker panel comprise TGFA and at least one more biomarker selected from IL6, S100A12, OSM, LSP1, MDK, CXCL9, HGF, CEACAM5, MMP12, KRT19, WFDC2, and PLAUR. In various embodiments, the biomarkers of a biomarker panel comprise S100A12 and at least one more biomarker selected from IL6, TGFA, OSM, LSP1, MDK, CXCL9, HGF, CEACAM5, MMP12, KRT19, WFDC2, and PLAUR. In various embodiments, the biomarkers of a biomarker panel comprise OSM and at least one more biomarker selected from IL6, TGFA, S100A12, LSP1, MDK, CXCL9, HGF, CEACAM5, MMP12, KRT19, WFDC2, and PLAUR. In various embodiments, the biomarkers of a biomarker panel comprise LSP1 and at least one more biomarker selected from IL6, TGFA, S100A12, OSM, MDK, CXCL9, HGF, CEACAM5, MMP12, KRT19, WFDC2, and PLAUR. In various embodiments, the biomarkers of a biomarker panel comprise MDK and at least one more biomarker selected from IL6, TGFA, S100A12, OSM, LSP1, CXCL9, HGF, CEACAM5, MMP12, KRT19, WFDC2, and PLAUR. In various embodiments, the biomarkers of a biomarker panel comprise CXCL9 and at least one more biomarker selected from IL6, TGFA, S100A12, OSM, LSP1, MDK, HGF, CEACAM5, MMP12, KRT19, WFDC2, and PLAUR. In various embodiments, the biomarkers of a biomarker panel comprise HGF and at least one more biomarker selected from IL6, TGFA, S100A12, OSM, LSP1, MDK, CXCL9, CEACAM5, MMP12, KRT19, WFDC2, and PLAUR. In various embodiments, the biomarkers of a biomarker panel comprise CEACAM5 and at least one more biomarker selected from IL6, TGFA, S100A12, OSM, LSP1, MDK, CXCL9, HGF, MMP12, KRT19, WFDC2, and PLAUR. In various embodiments, the biomarkers of a biomarker panel comprise MMP12 and at least one more biomarker selected from IL6, TGFA, S100A12, OSM, LSP1, MDK, CXCL9, HGF, CEACAM5, KRT19, WFDC2, and PLAUR. In various embodiments, the biomarkers of a biomarker panel comprise KRT19 and at least one more biomarker selected from IL6, TGFA, S100A12, OSM, LSP1, MDK, CXCL9, HGF, CEACAM5, MMP12, WFDC2, and PLAUR. In various embodiments, the biomarkers of a biomarker panel comprise WFDC2 and at least one more biomarker selected from IL6, TGFA, S100A12, OSM, LSP1, MDK, CXCL9, HGF, CEACAM5, MMP12, KRT19, and PLAUR. In various embodiments, the biomarkers of a biomarker panel comprise PLAUR and at least one more biomarker selected from IL6, TGFA, S100A12, OSM, LSP1, MDK, CXCL9, HGF, CEACAM5, MMP12, KRT19, and WFDC2.

In various embodiments, the plurality of biomarkers is selected from IL6, LSP1, MDK, MMP12; CEACAM5, IL6, MDK, MMP12, TGFA; HGF, IL6, MDK, MMP12, TGFA; CEACAM5, IL6, MDK, TGFA; IL6, MDK, MMP12, OSM; IL6, MDK, MMP12, TGFA; CEACAM5, IL6, LSP1, MDK, TGFA; HGF, IL6, MDK, MMP12, OSM; HGF, IL6, LSP1, MDK, MMP12; IL6, KRT19, MDK, MMP12, TGFA; HGF, IL6, LSP1, MDK; IL6, LSP1, MDK; IL6, LSP1, MDK, TGFA; IL6, MDK, TGFA; CXCL9, IL6, LSP1, MDK; CEACAM5, IL6, MDK, OSM, TGFA; CEACAM5, HGF, IL6, MDK, TGFA; CEACAM5, IL6, MDK, OSM; CEACAM5, IL6, MDK, MMP12, OSM; HGF, IL6, LSP1, MDK, TGFA; CEACAM5, IL6, LSP1, MDK; CEACAM5, IL6, MDK, S100A12, TGFA; HGF, IL6, LSP1, MDK, OSM; CEACAM5, HGF, IL6, MDK, OSM; IL6, LSP1, MDK, MMP12, TGFA; IL6, MDK, MMP12, OSM, TGFA; CEACAM5, IL6, MDK, TGFA, WFDC2; CXCL9, IL6, LSP1, MDK, MMP12; IL6, LSP1, MDK, MMP12, OSM; IL6, KRT19, LSP1, MDK, TGFA; IL6, LSP1, MDK, TGFA, WFDC2; CEACAM5, IL6, LSP1, MDK, MMP12; CEACAM5, IL6, MDK, PLAUR, TGFA; HGF, IL6, MDK, TGFA; or IL6, MDK, TGFA, WFDC2. In various embodiments, the plurality of biomarkers comprises IL6, LSP1, MDK, and MMP12. In various embodiments, the plurality of biomarkers comprises CEACAM5, IL6, MDK, MMP12, and TGFA. In various embodiments, the plurality of biomarkers comprises HGF, IL6, MDK, MMP12, and TGFA. In various embodiments, the plurality of biomarkers comprises CEACAM5, IL6, MDK, and TGFA. In various embodiments, the plurality of biomarkers comprises IL6, MDK, MMP12, and OSM. In various embodiments, the plurality of biomarkers comprises IL6, MDK, MMP12, and TGFA. In various embodiments, the plurality of biomarkers comprises CEACAM5, IL6, LSP1, MDK, and TGFA. In various embodiments, the plurality of biomarkers comprises HGF, IL6, MDK, MMP12, and OSM. In various embodiments, the plurality of biomarkers comprises HGF, IL6, LSP1, MDK, and MMP12. In various embodiments, the plurality of biomarkers comprises IL6, KRT19, MDK, MMP12, and TGFA. In various embodiments, the plurality of biomarkers comprises HGF, IL6, LSP1, and MDK. In various embodiments, the plurality of biomarkers comprises IL6, LSP1, and MDK. In various embodiments, the plurality of biomarkers comprises IL6, LSP1, MDK, and TGFA. In various embodiments, the plurality of biomarkers comprises IL6, MDK, and TGFA. In various embodiments, the plurality of biomarkers comprises CXCL9, IL6, LSP1, and MDK. In various embodiments, the plurality of biomarkers comprises CEACAM5, IL6, MDK, OSM, and TGFA. In various embodiments, the plurality of biomarkers comprises CEACAM5, HGF, IL6, MDK, and TGFA. In various embodiments, the plurality of biomarkers comprises CEACAM5, IL6, MDK, and OSM. In various embodiments, the plurality of biomarkers comprises CEACAM5, IL6, MDK, MMP12, and OSM. In various embodiments, the plurality of biomarkers comprises HGF, IL6, LSP1, MDK, and TGFA. In various embodiments, the plurality of biomarkers comprises CEACAM5, IL6, LSP1, and MDK. In various embodiments, the plurality of biomarkers comprises CEACAM5, IL6, MDK, S100A12, and TGFA. In various embodiments, the plurality of biomarkers comprises HGF, IL6, LSP1, MDK, and OSM. In various embodiments, the plurality of biomarkers comprises CEACAM5, HGF, IL6, MDK, and OSM. In various embodiments, the plurality of biomarkers comprises IL6, LSP1, MDK, MMP12, and TGFA. In various embodiments, the plurality of biomarkers comprises IL6, MDK, MMP12, OSM, and TGFA. In various embodiments, the plurality of biomarkers comprises CEACAM5, IL6, MDK, TGFA, and WFDC2. In various embodiments, the plurality of biomarkers comprises CXCL9, IL6, LSP1, MDK, and MMP12. In various embodiments, the plurality of biomarkers comprises IL6, LSP1, MDK, MMP12, and OSM. In various embodiments, the plurality of biomarkers comprises IL6, KRT19, LSP1, MDK, and TGFA. In various embodiments, the plurality of biomarkers comprises IL6, LSP1, MDK, TGFA, and WFDC2. In various embodiments, the plurality of biomarkers comprises CEACAM5, IL6, LSP1, MDK, and MMP12. In various embodiments, the plurality of biomarkers comprises CEACAM5, IL6, MDK, PLAUR, and TGFA. In various embodiments, the plurality of biomarkers comprises HGF, IL6, MDK, and TGFA. In various embodiments, the plurality of biomarkers comprises IL6, MDK, TGFA, and WFDC2.

In various embodiments, the biomarkers of a biomarker panel comprise IL6 and MDK, and at least one more biomarker selected from MMP12, LSP1, CEACAM5, HGF, OSM, and KRT19. In various embodiments, the plurality of biomarkers comprises IL6, LSP1, MDK, and MMP12. In various embodiments, the plurality of biomarkers comprises CEACAM5, IL6, MDK, MMP12, and TGFA. In various embodiments, the plurality of biomarkers comprises HGF, IL6, MDK, MMP12, and TGFA. In various embodiments, the plurality of biomarkers comprises CEACAM5, IL6, MDK, and TGFA. In various embodiments, the plurality of biomarkers comprises IL6, MDK, MMP12, and OSM. In various embodiments, the plurality of biomarkers comprises IL6, MDK, MMP12, and TGFA. In various embodiments, the plurality of biomarkers comprises CEACAM5, IL6, LSP1, MDK, and TGFA. In various embodiments, the plurality of biomarkers comprises HGF, IL6, MDK, MMP12, and OSM. In various embodiments, the plurality of biomarkers comprises HGF, IL6, LSP1, MDK, and MMP12. In various embodiments, the plurality of biomarkers comprises IL6, KRT19, MDK, MMP12, and TGFA.

In various embodiments, the plurality of biomarkers comprise three or more of TGFA, S100A12, OSM, TFPI2, LSP1, MDK, CXCL9, CLEC4D, IL6, ALPP, HGF, VWA1, CEACAM5, MMP12, KRT19, CASP8, WFDC2, and PLAUR. In various embodiments, the plurality of biomarkers comprise four or more, five or more, six or more, seven or more, eight or more, nine or more, ten or more, eleven or more, twelve or more, thirteen or more, fourteen or more, fifteen or more, sixteen or more, or seventeen or more of TGFA, S100A12, OSM, TFPI2, LSP1, MDK, CXCL9, CLEC4D, IL6, ALPP, HGF, VWA1, CEACAM5, MMP12, KRT19, CASP8, WFDC2, and PLAUR. In various embodiments, the plurality of biomarkers comprise each of TGFA, S100A12, OSM, TFPI2, LSP1, MDK, CXCL9, CLEC4D, IL6, ALPP, HGF, VWA1, CEACAM5, MMP12, KRT19, CASP8, WFDC2, and PLAUR. In various embodiments, the plurality of biomarkers consist of TGFA, S100A12, OSM, TFPI2, LSP1, MDK, CXCL9, CLEC4D, IL6, ALPP, HGF, VWA1, CEACAM5, MMP12, KRT19, CASP8, WFDC2, and PLAUR.

In various embodiments, the plurality of biomarkers comprise three or more of TGFA, S100A12, OSM, TFPI2, LSP1, MDK, CXCL9, CLEC4D, IL6, HGF, VWA1, CEACAM5, MMP12, KRT19, CASP8, WFDC2, and PLAUR. In various embodiments, the biomarkers of a biomarker panel comprise IL6, MDK, and TGFA, and at least one more biomarker selected from S100A12, OSM, TFPI2, LSP1, CXCL9, CLEC4D, ALPP, HGF, VWA1, CEACAM5, MMP12, KRT19, CASP8, WFDC2, and PLAUR. In various embodiments, the biomarkers of a biomarker panel comprise IL6, MDK, and S100A12, and at least one more biomarker selected from TGFA, OSM, TFPI2, LSP1, CXCL9, CLEC4D, ALPP, HGF, VWA1, CEACAM5, MMP12, KRT19, CASP8, WFDC2, and PLAUR. In various embodiments, the biomarkers of a biomarker panel comprise IL6, MDK, and OSM, and at least one more biomarker selected from TGFA, S100A12, TFPI2, LSP1, CXCL9, CLEC4D, ALPP, HGF, VWA1, CEACAM5, MMP12, KRT19, CASP8, WFDC2, and PLAUR. In various embodiments, the biomarkers of a biomarker panel comprise IL6, MDK, and TFPI2, and at least one more biomarker selected from TGFA, S100A12, OSM, LSP1, CXCL9, CLEC4D, ALPP, HGF, VWA1, CEACAM5, MMP12, KRT19, CASP8, WFDC2, and PLAUR. In various embodiments, the biomarkers of a biomarker panel comprise IL6, MDK, and LSP1, and at least one more biomarker selected from TGFA, S100A12, OSM, TFPI2, CXCL9, CLEC4D, ALPP, HGF, VWA1, CEACAM5, MMP12, KRT19, CASP8, WFDC2, and PLAUR. In various embodiments, the biomarkers of a biomarker panel comprise IL6, MDK, and CXCL9, and at least one more biomarker selected from TGFA, S100A12, OSM, TFPI2, LSP1, CLEC4D, ALPP, HGF, VWA1, CEACAM5, MMP12, KRT19, CASP8, WFDC2, and PLAUR. In various embodiments, the biomarkers of a biomarker panel comprise IL6, MDK, and CLEC4D, and at least one more biomarker selected from TGFA, S100A12, OSM, TFPI2, LSP1, CXCL9, ALPP, HGF, VWA1, CEACAM5, MMP12, KRT19, CASP8, WFDC2, and PLAUR. In various embodiments, the biomarkers of a biomarker panel comprise IL6, MDK, and ALPP, and at least one more biomarker selected from TGFA, S100A12, OSM, TFPI2, LSP1, CXCL9, CLEC4D, HGF, VWA1, CEACAM5, MMP12, KRT19, CASP8, WFDC2, and PLAUR. In various embodiments, the biomarkers of a biomarker panel comprise IL6, MDK, and HGF, and at least one more biomarker selected from TGFA, S100A12, OSM, TFPI2, LSP1, CXCL9, CLEC4D, ALPP, VWA1, CEACAM5, MMP12, KRT19, CASP8, WFDC2, and PLAUR. In various embodiments, the biomarkers of a biomarker panel comprise IL6, MDK, and VWA1, and at least one more biomarker selected from TGFA, S100A12, OSM, TFPI2, LSP1, CXCL9, CLEC4D, ALPP, HGF, CEACAM5, MMP12, KRT19, CASP8, WFDC2, and PLAUR. In various embodiments, the biomarkers of a biomarker panel comprise IL6, MDK, and CEACAM5, and at least one more biomarker selected from TGFA, S100A12, OSM, TFPI2, LSP1, CXCL9, CLEC4D, ALPP, HGF, VWA1, MMP12, KRT19, CASP8, WFDC2, and PLAUR. In various embodiments, the biomarkers of a biomarker panel comprise IL6, MDK, and MMP12, and at least one more biomarker selected from TGFA, S100A12, OSM, TFPI2, LSP1, CXCL9, CLEC4D, ALPP, HGF, VWA1, CEACAM5, KRT19, CASP8, WFDC2, and PLAUR. In various embodiments, the biomarkers of a biomarker panel comprise IL6, MDK, and KRT19, and at least one more biomarker selected from TGFA, S100A12, OSM, TFPI2, LSP1, CXCL9, CLEC4D, ALPP, HGF, VWA1, CEACAM5, MMP12, CASP8, WFDC2, and PLAUR. In various embodiments, the biomarkers of a biomarker panel comprise IL6, MDK, and CASP8, and at least one more biomarker selected from TGFA, S100A12, OSM, TFPI2, LSP1, CXCL9, CLEC4D, ALPP, HGF, VWA1, CEACAM5, MMP12, KRT19, WFDC2, and PLAUR. In various embodiments, the biomarkers of a biomarker panel comprise IL6, MDK, and WFDC2, and at least one more biomarker selected from TGFA, S100A12, OSM, TFPI2, LSP1, CXCL9, CLEC4D, ALPP, HGF, VWA1, CEACAM5, MMP12, KRT19, CASP8, and PLAUR. In various embodiments, the biomarkers of a biomarker panel comprise IL6, MDK, and PLAUR, and at least one more biomarker selected from TGFA, S100A12, OSM, TFPI2, LSP1, CXCL9, CLEC4D, ALPP, HGF, VWA1, CEACAM5, MMP12, KRT19, CASP8, and WFDC2.

In various embodiments, the plurality of biomarkers comprise four or more, five or more, six or more, seven or more, eight or more, nine or more, ten or more, eleven or more, twelve or more, thirteen or more, fourteen or more, fifteen or more, or sixteen or more of TGFA, S100A12, OSM, TFPI2, LSP1, MDK, CXCL9, CLEC4D, IL6, HGF, VWA1, CEACAM5, MMP12, KRT19, CASP8, WFDC2, and PLAUR. In various embodiments, the plurality of biomarkers comprise each of TGFA, S100A12, OSM, TFPI2, LSP1, MDK, CXCL9, CLEC4D, IL6, HGF, VWA1, CEACAM5, MMP12, KRT19, CASP8, WFDC2, and PLAUR. In various embodiments, the plurality of biomarkers consist of TGFA, S100A12, OSM, TFPI2, LSP1, MDK, CXCL9, CLEC4D, IL6, HGF, VWA1, CEACAM5, MMP12, KRT19, CASP8, WFDC2, and PLAUR.

In various embodiments, the plurality of biomarkers comprises CEACAM5, HGF, IL6, MDK, MMP12, OSM, PLAUR, and TGFA. In various embodiments, the plurality of biomarkers comprises CEACAM5, CXCL9, HGF, IL6, LSP1, MDK, MMP12, and TGFA. In various embodiments, the plurality of biomarkers comprises CEACAM5, HGF, IL6, KRT19, LSP1, MDK, PLAUR, and TGFA. In various embodiments, the plurality of biomarkers comprises CEACAM5, HGF, IL6, LSP1, MDK, OSM, PLAUR, and TGFA. In various embodiments, the plurality of biomarkers comprises CEACAM5, HGF, IL6, LSP1, MDK, MMP12, PLAUR, and TGFA. In various embodiments, the plurality of biomarkers comprises CEACAM5, CXCL9, HGF, IL6, LSP1, MDK, MMP12, PLAUR, S100A12, and TGFA. In various embodiments, the plurality of biomarkers comprises CEACAM5, HGF, IL6, LSP1, MDK, MMP12, OSM, PLAUR, S100A12, and TGFA. In various embodiments, the plurality of biomarkers comprises CEACAM5, CXCL9, HGF, IL6, KRT19, LSP1, MDK, MMP12, TGFA, and WFDC2. In various embodiments, the plurality of biomarkers comprises CEACAM5, CXCL9, HGF, IL6, KRT19, LSP1, MDK, MMP12, PLAUR, and TGFA. In various embodiments, the plurality of biomarkers comprises CEACAM5, HGF, IL6, MDK, MMP12, OSM, PLAUR, S100A12, TGFA, and WFDC2. In various embodiments, the plurality of biomarkers comprises CEACAM5, CXCL9, HGF, IL6, KRT19, LSP1, MDK, MMP12, OSM, PLAUR, S100A12, TFPI2, TGFA, VWA1, and WFDC2. In various embodiments, the plurality of biomarkers comprises CEACAM5, CLEC4D, CXCL9, HGF, IL6, KRT19, LSP1, MDK, MMP12, OSM, PLAUR, S100A12, TFPI2, TGFA, and WFDC2. In various embodiments, the plurality of biomarkers comprises CASP8, CEACAM5, CXCL9, HGF, IL6, KRT19, LSP1, MDK, MMP12, OSM, PLAUR, S100A12, TFPI2, TGFA, and VWA1. In various embodiments, the plurality of biomarkers comprises CASP8, CEACAM5, CXCL9, HGF, IL6, KRT19, LSP1, MDK, MMP12, OSM, PLAUR, TFPI2, TGFA, VWA1, and WFDC2. In various embodiments, the plurality of biomarkers comprises CEACAM5, CLEC4D, CXCL9, HGF, IL6, KRT19, LSP1, MDK, MMP12, OSM, PLAUR, S100A12, TGFA, VWA1, and WFDC2. In various embodiments, the plurality of biomarkers comprises CASP8, CEACAM5, CLEC4D, CXCL9, HGF, IL6, KRT19, LSP1, MDK, MMP12, OSM, PLAUR, S100A12, TFPI2, TGFA, VWA1, and WFDC2.

In various embodiments, the biomarkers of a biomarker panel comprise any combination of biomarkers as shown in Table 5. In various embodiments, the plurality of biomarkers comprises any combination of biomarkers as shown in Table 5.

### V. Assays

As shown in FIG. 1A, the system environment 100 involves implementing a marker quantification assay 120 for evaluating expression levels of one or more biomarkers. Examples of an assay (e.g., marker quantification assay 120) for one or more markers include DNA assays, microarrays, polymerase chain reaction (PCR), RT-PCR, Southern blots, Northern blots, antibody-binding assays, enzyme-linked immunosorbent assays (ELISAs), flow cytometry, protein assays, Western blots, nephelometry, turbidimetry, chromatography, mass spectrometry, immunoassays, including, by way of example, but not limitation, RIA, immunofluorescence, immunochemiluminescence, immunoelectrochemiluminescence, or competitive immunoassays, immunoprecipitation, and the assays described in the Examples section below. The information from the assay can be quantitative and sent to a computer system of the invention. The information can also be qualitative, such as observing patterns or fluorescence, which can be translated into a quantitative measure by a user or automatically by a reader or computer system.

Various immunoassays designed to quantitate markers can be used in screening including multiplex assays (e.g., an assay which simultaneously measures multiple analytes in a single cycle of the assay). Measuring the concentration of a target marker in a sample or fraction thereof can be accomplished by a variety of specific assays. For example, a conventional sandwich type assay can be used in an array, ELISA, RIA, etc. format. Other immunoassays include Ouchterlony plates that provide a simple determination of antibody binding. Additionally, Western blots can be performed on protein gels or protein spots on filters, using a detection system specific for the markers as desired, conveniently using a labeling method.

Protein based analysis, using an antibody that specifically binds to a polypeptide (e.g. marker), can be used to quantify the marker level in a test sample obtained from a subject. In various embodiments, an antibody that binds to a marker can be a monoclonal antibody. In various embodiments, an antibody that binds to a marker can be a polyclonal antibody. In various embodiments, both monoclonal and polyclonal antibodies are used to bind polypeptides for the protein based analysis.

For multiplex analysis of markers, arrays containing one or more marker affinity reagents, e.g. antibodies can be generated. Such an array can be constructed comprising antibodies against markers. Detection can utilize one or a panel of marker affinity reagents, e.g. a panel or cocktail of affinity reagents specific for one, two, three, four, five, six, seven, eight, nine, ten, eleven, twelve, thirteen, fourteen, fifteen, sixteen, seventeen, eighteen, nineteen, twenty, twenty one, or more markers.

In various embodiments, the multiplex assay involves the use of oligonucleotide labeled antibody probes that bind to target biomarkers and allow for subsequent quantification of biomarkers. One example of a multiplex assay that involves oligonucleotide labeled antibody probes is the Proximity Extension Assay (PEA) technology (Olink Proteomics). Briefly, a pair of oligonucleotide labeled antibodies bind to a biomarker, wherein the two oligonucleotide sequences are complementary to one another. Thus, when both antibodies bind to the target biomarker, the oligonucleotide sequences hybridize with one another. Mismatched oligonucleotide sequences (which occurs due to non-specific binding of antibodies or cross-reactivity of antibodies) will not hybridize and therefore, will not result in a readout. Hybridized oligonucleotide sequences undergo nucleic acid extension and amplification, followed by quantification using microfluidic qPCR. The quantified levels correlate to the quantitative expression values of the respective biomarkers. Further details of the Olink Proximity Extension Assay (PEA) is described in Wik, L., et al. (2021). Proximity Extension Assay in Combination with Next-Generation Sequencing for High-throughput Proteome-wide Analysis. Molecular & cellular proteomics: MCP, 20, 100168, which is hereby incorporated by reference in its entirety.

In various embodiments, the multiplex assay involves the use of bead conjugated antibodies (e.g., capture antibodies) that enable the binding and detection of biomarkers. One example of a multiplex assay involving bead conjugated antibodies is Luminex's xMAP® Technology. Here, bead conjugated antibodies are added to the sample along with biotinylated detection antibodies. Both antibodies are specific to the biomarkers of interest and therefore, form an antibody-antigen sandwich. Streptavidin is further added, which binds to the biotinylated detection antibodies and enables detection of the complex. The Luminex 200™ or FlexMap® analyzer are employed to identify and quantify the amount of the biomarker in the sample. In various embodiments, the multiplex assay represents an improvement over Luminex's xMAP® technology, such as the Multi-Analyte Profile (MAP) technology by Myriad Rules Based Medicine (RBM), Inc.

In various embodiments, the multiplex assay involves the use of single molecule array (SIMOA) testing. For example, the assay may use paramagnetic particles coupled with antibodies that exhibit binding specificity to specific protein biomarkers. Detection antibodies are added which bind with the protein biomarkers to form fluorescent products. Thus, immunocomplexes including the paramagnetic bead, bound protein biomarker, and detection antibody are generated. Immunocomplexes are loaded into arrays (e.g., microarrays) in which individual immunocomplexes are separately localized. Next, enzymatic signal amplification occurs and fluorescent imaging is performed to capture the read out from the respective immunocomplexes in the microarray. This enables detection and/or quantification of individual protein biomarkers that were present in the sample. An example of such a multiplex assay is the SIMOA Bead-based assay from Quanterix™.

In various embodiments, the multiplex assay involves performing mass spectrometry based protein/peptide measurements. For example, in one embodiment, nanoparticles are engineered with surface physicochemical properties which enable protein biomarker binding to the surface of the magnetic nanoparticles. Here, a protein corona is formed on the surface of the nanoparticle composed of varying biomarker proteins. Nanoparticles can be synthesized with varying surface physicochemical properties to achieve differing protein coronas. Nanoparticle protein corona purification is performed using a magnet and corona proteins are digested. Mass spectrometry e.g., LC-MS/MS can be performed to determine presence and/or quantity of protein/peptide biomarkers. An example of such a multiplex assay is the Seer Proteograph Assay kit using the SP100 Automation Instrument for analyzing protein biomarkers. Further details of profiling proteomes using nanoparticle protein coronas is described in Blume, J. et al, “Rapid, deep and precise profiling of the plasma proteome with multi-nanoparticle protein corona.” Nat Commun 11, 3662 (2020), which is hereby incorporated by reference in its entirety.

In various embodiments, the multiplex assay involves using an aptamer based approach. For example, the assay can use chemically modified aptamers for detecting and discovering protein biomarkers. For example, modified aptamer reagents are synthesized with a fluorophore, cleavable linker, and biotin molecule. The modified aptamer can bind and capture protein biomarkers, while the biotin molecule binds to a corresponding streptavidin bead. Bound protein biomarkers are further tagged with biotin molecules and the cleavable linker is cleaved to release the protein biomarker—aptamer conjugate from the streptavidin bead. A polyanionic competitor is added to prevent rebinding of non-specific complexes. Protein biomarkers are recaptured on streptavidin beads via the biotin molecule and fluorophores are measured to read out protein biomarker presence/quantity. An example of such a multiplex assay is the SOMAscan® assay. Further details of the SOMAscan® assay is described in Gold, L., et al., (20-10). Aptamer-based multiplexed proteomic technology for biomarker discovery. PloS one, 1(12), e 5004, which is hereby incorporated by reference in its entirety.

In various embodiments, prior to implementation of a marker quantification assay 120 (e.g., a multiplex assay), a sample obtained from a subject can be processed. In various embodiments, processing the sample enables the implementation of the marker quantification assay 120 to more accurately evaluate expression levels of one or more biomarkers in the sample.

In various embodiments, the sample from a subject can be processed to extract biomarkers from the sample. In one embodiment, the sample can undergo phase separation to separate the biomarkers from other portions of the sample. For example, the sample can undergo centrifugation (e.g., pelleting or density gradient centrifugation) to separate larger and/or more dense entities in the sample (e.g., cells and other macromolecules) from the biomarkers. Other examples include filtration (e.g., ultrafiltration) to phase separate the biomarkers from other portions of the sample.

In various embodiments, the sample from a subject can be processed to produce a sub-sample with a fraction of biomarkers that were in the sample. In various embodiments, producing a fraction of biomarkers can involve performing a protein fractionation procedure. One example of protein fractionation procedures include chromatography (e.g., gel filtration, ion exchange, hydrophobic chromatography, or affinity chromatography). In particular embodiments, the protein fractionation procedure involves affinity purification or immunoprecipitation where biomarkers are bound by specific antibodies. Such antibodies can be immobilized on a support, such as a magnetic particle or nanoparticle or a plate.

In various embodiments, the sample from the subject is processed to extract biomarkers from the sample and further processed to produce a sub-sample with a fraction of extracted biomarkers. Altogether, this enables a purified sub-sample of biomarkers that are of particular interest. Thus, implementing an assay (e.g., an immunoassay) for evaluating expression levels of the biomarkers of particular interest can be more accurate and of higher quality. In various embodiments, the biomarkers of particular interest can be biomarkers of a biomarker panel, embodiments of which are described herein. In various embodiments, the biomarkers include the biomarkers shown in Table 2, and Table 3, and combinations of biomarkers shown in Table 4, and Table 5.

### VI. Example Cancers

Methods described herein involve implementing biomarker panels for generating a cancer prediction, such as a prediction of presence or absence of cancer (e.g., early stage cancer or non-early stage cancer). In various embodiments, the biomarker panels described herein are implemented to predict presence or absence of a cancer, such as a lung cancer. In various embodiments, the biomarker panels described herein are implemented to generate a prediction informative for early detection of a cancer, such as an early stage lung cancer or non-early stage lung cancer.

In various embodiments, the cancer is a lung cancer. In some embodiments, the lung cancer is an adenocarcinoma, an adenosquamous cell cancer, a large cell cancer, a neuroendocrine cancer, a non-small cell lung cancer (NSCLC), a small cell cancer, or a squamous cell cancer. In some embodiments, the lung cancer is an adenocarcinoma. In some embodiments, the lung cancer is an adenosquamous cell cancer. In some embodiments, the lung cancer is a large cell cancer. In some embodiments, the lung cancer is a neuroendocrine cancer. In some embodiments, the lung cancer is a non-small cell lung cancer (NSCLC). In some embodiments, the lung cancer is a small cell cancer. In some embodiments, the lung cancer is a squamous cell cancer.

In various embodiments, biomarker panels described herein generate a cancer prediction for a particular stage of lung cancer, such as a stage 0, stage 1, stage 2, stage 3, or stage 4 lung cancer. In particular embodiments, biomarker panels disclosed herein are useful for generating a cancer prediction informative for early detection of lung cancer, such as early detection of the lung cancer while the lung cancer is a stage 0, stage 1, stage 2. In various embodiments, biomarker panels described herein generate a cancer prediction for a particular subtype of lung cancer, including any one of adenocarcinoma, squamous lung cancer, neuroendocrine, small cell lung cancer, non-small cell lung cancer, large cell lung cancer, or adenosquamous carcinoma.

In various embodiments, any method, non-transitory computer readable medium, system, or kit provided herein optionally comprises administering a treatment to the subject. In various embodiments, the treatment comprises a surgery, a chemotherapy, a radiation therapy, a targeted therapy, or any combination thereof. In various embodiments, the treatment comprises a surgery. In various embodiments, the treatment comprises a chemotherapy. In various embodiments, the treatment comprises a radiation therapy. In various embodiments, the treatment comprises a targeted therapy.

In various embodiments, the methods disclosed herein optionally comprise administering a treatment to the subject. In various embodiments, the non-transitory computer readable medium disclosed herein optionally comprises administering a treatment to the subject. In various embodiments, the systems disclosed herein optionally comprise administering a treatment to the subject. In various embodiments, the kits disclosed herein optionally comprise administering a treatment to the subject. In various embodiments, the treatment comprises a surgery, a chemotherapy, a radiation therapy, a targeted therapy, or any combination thereof. In various embodiments, the treatment comprises a surgery. In various embodiments, the treatment comprises a chemotherapy. In various embodiments, the treatment comprises a radiation therapy. In various embodiments, the treatment comprises a targeted therapy.

In various embodiments, the methods disclosed herein optionally comprise administering a treatment to the subject, wherein the treatment comprises a surgery, a chemotherapy, a radiation therapy, a targeted therapy, immunotherapy, or any combination thereof. In various embodiments, the non-transitory computer readable medium disclosed herein optionally comprises administering a treatment to the subject, wherein the treatment comprises a surgery, a chemotherapy, a radiation therapy, a targeted therapy, immunotherapy, or any combination thereof. In various embodiments, the systems disclosed herein optionally comprise administering a treatment to the subject, wherein the treatment comprises a surgery, a chemotherapy, a radiation therapy, a targeted therapy, immunotherapy, or any combination thereof. In various embodiments, the kits disclosed herein optionally comprise administering a treatment to the subject, wherein the treatment comprises a surgery, a chemotherapy, a radiation therapy, a targeted therapy, immunotherapy, or any combination thereof.

### VII. Computer Implementation

The methods disclosed herein, such as the methods of generating a prediction of cancer in a subject, are, in some embodiments, performed on one or more computers. For example, the building and deployment of a predictive model to analyze expression levels of a plurality of biomarkers, and database storage can be implemented in hardware or software, or a combination of both. In one embodiment of the invention, a machine-readable storage medium is provided, the medium comprising a data storage material encoded with machine readable data which, when using a machine programmed with instructions for using said data, is capable of displaying any of the datasets and execution and results of a predictive model of this invention. Such data can be used for a variety of purposes, such as patient monitoring, treatment considerations, and the like. The invention can be implemented in computer programs executing on programmable computers, comprising a processor, a data storage system (including volatile and non-volatile memory and/or storage elements), a graphics adapter, a pointing device, a network adapter, at least one input device, and at least one output device. Program code may be applied to input data to perform the functions described above and generate output information. The output information is applied to one or more output devices, in known fashion. The computer can be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

FIG. 3 illustrates an example computer 300 for implementing the entities shown in FIGS. 1A, 1B, and 2. The computer 300 includes at least one processor 302 coupled to a chipset 304. The chipset 304 includes a memory controller hub 320 and an input/output (I/O) controller hub 322. A memory 306 and a graphics adapter 312 are coupled to the memory controller hub 320, and a display 318 is coupled to the graphics adapter 312. A storage device 308, an input device 314, and network adapter 316 are coupled to the I/O controller hub 322. Other embodiments of the computer 300 have different architectures.

The storage device 308 is a non-transitory computer-readable storage medium such as a hard drive, compact disk read-only memory (CD-ROM), DVD, or a solid-state memory device. The memory 306 holds instructions and data used by the processor 302. The input device 314 is a touch-screen interface, a mouse, track ball, or other type of pointing device, a keyboard, or some combination thereof, and is used to input data into the computer 300. In some embodiments, the computer 300 may be configured to receive input (e.g., commands) from the input device 314 via gestures from the user. The graphics adapter 312 displays images and other information on the display 318. The network adapter 316 couples the computer 300 to one or more computer networks.

The computer 300 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 308, loaded into the memory 306, and executed by the processor 302.

The types of computers 300 used by the entities of FIG. 1A can vary depending upon the embodiment and the processing power required by the entity. For example, the can run in a single computer 300 or multiple computers 300 communicating with each other through a network such as in a server farm. The computers 300 can lack some of the components described above, such as graphics adapters 312, and displays 318.

### VIII. Kit Implementation

Also disclosed herein are kits for generating a cancer prediction (e.g., a prediction of presence or absence of cancer in a subject). Such kits can include reagents for detecting expression levels of one or biomarkers and instructions for generating the cancer prediction based on the detected expression levels.

In various embodiments, the detection reagents can be provided as part of a kit. Thus, the invention further provides kits for detecting the presence of a panel of biomarkers of interest in a biological test sample. A kit can comprise a set of reagents for generating a dataset via at least one protein detection assay (e.g., a multiplex assay such as a Proximity Extension Assay (PEA)) that analyzes the test sample from the subject. In various embodiments, the set of reagents enable detection of quantitative expression levels of any of the biomarkers detailed in Table 2. In particular embodiments, the set of reagents enable detection of quantitative expression levels of any of the biomarker combinations detailed in Table 3. In particular embodiments, the set of reagents enable detection of quantitative expression levels of any of the biomarker combinations detailed in Table 4. In particular embodiments, the set of reagents enable detection of quantitative expression levels of any of the biomarker combinations detailed in Table 5. In certain aspects, the reagents include one or more antibodies that bind to one or more of the markers. The antibodies may be monoclonal antibodies, polyclonal antibodies, or both monoclonal and polyclonal antibodies. In some aspects, the reagents can include reagents for performing an ELISA including buffers and detection agents.

A kit can include instructions for use of a set of reagents. For example, a kit can include instructions for performing at least one biomarker detection assay such as an immunoassay (e.g., a multiplex assay such as a Proximity Extension Assay (PEA)), a protein-binding assay, an antibody-based assay, an antigen-binding protein-based assay, a protein-based array, an enzyme-linked immunosorbent assay (ELISA), flow cytometry, a protein array, a blot, a Western blot, nephelometry, turbidimetry, chromatography, mass spectrometry, enzymatic activity, proximity extension assay, and an immunoassay selected from RIA, immunofluorescence, immunochemiluminescence, immunoelectrochemiluminescence, immunoelectrophoretic, a competitive immunoassay, and immunoprecipitation.

In various embodiments, the kits include instructions for practicing the methods disclosed herein (e.g., methods for training or deploying a predictive model to analyze biomarker expression levels to generate a cancer prediction). These instructions can be present in the subject kits in a variety of forms, one or more of which can be present in the kit. One form in which these instructions can be present is as printed information on a suitable medium or substrate, e.g., a piece or pieces of paper on which the information is printed, in the packaging of the kit, in a package insert, etc. Yet another means would be a computer readable medium, e.g., diskette, CD, hard-drive, network data storage, etc., on which the information has been recorded. Yet another means that can be present is a website address which can be used via the internet to access the information at a removed site. Any convenient means can be present in the kits.

### IX. Systems

Further disclosed herein are system for analyzing quantitative expression levels of biomarkers for generating a cancer prediction (e.g., a prediction of presence or absence of cancer in a subject). In various embodiments, such a system can include a set of reagents for detecting expression levels of biomarkers in the biomarker panel, an apparatus configured to receive a mixture of the set of reagents and a test sample obtained from a subject to measure the expression levels of the biomarkers, and a computer system communicatively coupled to the apparatus to obtain the measured expression levels and to implement the predictive model to analyze the expression levels to generate a cancer prediction (e.g., a prediction of presence or absence of cancer in the subject).

The set of reagents enable the detection of quantitative expression levels of the biomarkers in the biomarker panel. In various embodiments, the set of reagents involve reagents used to perform an assay, such as an assay or immunoassay as described above. For example, the reagents include one or more antibodies that bind to one or more of the biomarkers. The antibodies may be monoclonal antibodies, polyclonal antibodies, or both monoclonal and polyclonal antibodies. As another example, the reagents can include reagents for performing ELISA including buffers and detection agents.

The apparatus is configured to detect expression levels of biomarkers in a mixture of a reagent and test sample. For example, the apparatus can determine quantitative expression levels of biomarkers through an immunologic assay or assay for nucleic acid detection. The mixture of the reagent and test sample may be presented to the apparatus through various conduits, examples of which include wells of a well plate (e.g., 96 well plate), a vial, a tube, and integrated fluidic circuits. As such, the apparatus may have an opening (e.g., a slot, a cavity, an opening, a sliding tray) that can receive the container including the reagent test sample mixture and perform a reading to generate quantitative expression values of biomarkers. Examples of an apparatus include a plate reader (e.g., a luminescent plate reader, absorbance plate reader, fluorescence plate reader), a spectrometer, and a spectrophotometer.

The computer system, such as example computer 300 described in FIG. 3, communicates with the apparatus to receive the quantitative expression values of biomarkers. The computer system implements, in silico, a predictive model to analyze the quantitative expression values of the biomarkers to generate a cancer prediction (e.g., presence or absence of cancer in a subject).

### X. Additional Embodiments

In various embodiments, disclosed herein is a method for predicting presence or absence of cancer in a subject, the method comprising: obtaining or having obtained a dataset comprising expression levels of a plurality of biomarkers from the subject, wherein the plurality of biomarkers comprise two or more biomarkers of NTF3, C3, OLR1, MMP9, CSF1, OSM, TNFRSF1A, WFDC2, CLEC5A, BHMT2, PLAUR, TGFA, GLI2, MMP8, LTBR, CXCL8, CD14, SHISA5, CD59, NPDC1, CXCL9, CCL23, COL4A1, PGF, GDF15, COL18A1, NCR3LG1, CXCL12, HAVCR2, HIP1R, RBP7, SPINT1, LTBP2, CALB1, RBFOX3, OCLN, GFRA1, FSTL3, EFNA1, BSG, LRG1, RELT, FGA, ITIH3, TIMP1, TNFRSF1B, CEACAM8, MAMDC2, IL6, FOLR1, CEACAM5, SPP1, CAPG, LGALS9, NPC2, IFI30, ELN, MMP12, VSIG4, NECTIN2, MAD1L1, EDA2R, TNFRSF10B, SMNDC1, PRSS8, CXCL17, PTPRF, TNFRSF10A, CSTB, TREM2, SDC1, DSC2, NME1, LMNB2, CKAP4, EPHB4, LAYN, DLL1, PRG2, SEZ6L2, COLEC12, ULBP2, B4GALT1, HAGH, LCN2, ATRAID, IL1RN, YAP1, TNFSF13, CST3, TNFRSF4, CCL18, POLR2F, EPHA2, SIRPB1, GM2A, SNRPB2, ITIH4, FBLN2, TNFRSF9, CDH2, IL18BP, CWC15, EFNA4, GFAP, ADAMTS16, CHGB, AREG, CCL14, CEACAM6, RNASE1, SPINK1, CD302, KLK7, NRP2, ITGBL1, PRTN3, AGRN, RCC1, THBS2, CRELD1, EFEMP1, SCARB2, C9, CHCHD10, EFHD1, FGL1, IL10RB, KLK4, SEPTIN8, TFF3, CRLF1, COL6A3, CPOX, ADAM8, C4BPB, CXCL16, LAIR1, SCARF2, SERPINB8, IL4R, CD276, CDH23, ANGPT2, ACVRL1, CTSV, GALNT5, RANBP2, VASN, VWA1, RNASE6, APOA2, ICAM1, IL2RA, ZBTB17, OSMR, GRPEL1, IGFBP4, VCAM1, AZU1, CTSD, RNASET2, CD93, SUSD5, SLAMF8, CCL26, IGFBP2, RNF149, MERTK, S100A11, SNED1, CEACAM21, UHRF2, CNDP1, NECTIN4, PIGR, SPRED2, VIPR1, FUT3_FUT5, S100A12, TNFRSF11B, IFNGR1, NPM1, ACTA2, KRT19, SIGLEC5, LAMP3, ALCAM, CD74, PRRT3, ITGA5, TGOLN2, CDCP1, CKB, S100P, SERPINA11, PILRA, NXA1, SLC4A1, NCF2, PTX3, LSP1, CD300A, CLEC7A, LPCAT2, NRP1, CHCHD6, SERPINA3, TNFRSF21, CTSC, LILRB4, NBN, CD55, B2M, ARG1, NGFR, PSMD1, SRP14, ITGB6, AMPD3, CD300E, PKD2, STC2, GCHFR, PGLYRP1, PILRB, CDH3, NMRK2, SMAD1, DCBLD2, CRIM1, HS6ST2, TNFRSF8, CYP24A1, BID, GLRX, TNFRSF14, DPEP2, F9, PTGDS, C2, ERMAP, IGFBPL1, CST1, ELOA, MUC13, IL1R1, S100A3, PIK3IP1, VNN2, TPMT, ANGPTL3, ASGR1, BMP4, CLEC4D, HSPG2, CCL3, CD300LF, COL28A1, CXCL10, QPCT, TGFBR2, COL24A1, CDH6, CD300C, FST, MYBPC2, KCTD5, CSF3, EBI3_IL27, SLC39A14, IL7, CA1, TOR1AIP1, CHI3L1, DGCR6, TNC, CLEC4G, CLPS, ENO3, EPN1, PTPRN2, ADM, LTA4H, TCOF1, TIMD4, CCL28, KLK11, KLK6, LYVE1, TGM2, FRZB, ADAM9, AHSP, CCL2, EGLN1, MRC1, MTUS1, RPS10, TACSTD2, SAA4, SLITRK6, CIT, TNFRSF19, IMMT, ORM1, CTHRC1, KIAA0319, BTN2A1, A1BG, DRAXIN, FGF6, SEMA3F, STC1, BCAM, BAP18, CCL16, DKK3, PODXL2, VWF, FAM20A, DENR, IGFBP7, MSTN, ENOPH1, TSPAN1, EFCAB14, AMBP, C1RL, IL5, TNFSF14, HAVCR1, TNFRSF12A, COL3A1, GPKOW, MANSC1, SEL1L, POSTN, GIPC2, DAPP1, DCN, FAS, GLYR1, LCN15, NEFL, USP28, CHAD, CRH, PBLD, PCNA, CSF2, PBK, BDNF, ROR1, FCN1, ANGPTL4, ZNRD2, CX3CL1, MYH7B, NADK, RAB44, TNFRSF11A, TNFRSF6B, CLMP, HDAC8, IGSF8, PALM2, RECK, CLEC14A, FKBP1B, IL13RA1, WNT9A, C2CD2L, CCDC80, PLA2G2A, SART1, KIRREL2, CCL4, IL18R1, NEO1, FLRT2, TFPI2, LBR, ISLR2, LECT2, PPY, SERPINA1, VWC2, FAM3C, HMBS, LMNB1, PRSS22, CALCB, CCL7, CTSL, FOLR2, PSAP, SEMA7A, GALNT7, NT5C1A, FGFR4, MICB_MICA, BLVRB, BPIFB2, CCN3, GPRC5C, INPP5J, FGFR2, CD83, SCRG1, ALDH3A1, CYTL1, OSCAR, PHLDB1, TNFSF11, GHRL, RRM2, ADGRG1, AXL, CA14, CFH, IL6R, LGALS3, SPON2, CAPS, DCTPP1, MSR1, RARRES2, SCN3A, SORCS2, SCG2, CRYBB2, DNAJA4, LILRA5, REN, COCH, CLEC11A, CRHBP, FARSA, NPHS1, PRAME, PRDX2, CXCL13, ASGR2, BRK1, SCPEP1, GNAS, BCL2L15, C9orf40, CD101, CGB3_CGB5_CGB8, CTSZ, ESM1, CDH17, C5, PON1, OLFM4, OPTC, PALM, PNLIPRP1, PXN, SYNGAP1, MSMB, HEPH, NGRN, CGREF1, LILRB2, NRN1, BCAT2, HNRNPUL1, INSL4, MPO, and PPL; and generating a prediction of presence or absence of the cancer in the subject by applying a predictive model to the expression levels of the plurality of biomarkers.

In various embodiments, a performance of the predictive model is characterized by an area under the curve (AUC) of at least 0.75. In various embodiments, a performance of the predictive model is characterized by an area under the curve (AUC) of at least 0.80. In various embodiments, a performance of the predictive model is characterized by an area under the curve (AUC) of at least 0.85. In various embodiments, a performance of the predictive model is characterized by an area under the curve (AUC) of at least 0.86. In various embodiments, a performance metric of the predictive model is improved in comparison to a model solely incorporating CEA (e.g., a cancer marker in common use today).

In various embodiments, the plurality of biomarkers comprise LTBR and at least a second biomarker. In various embodiments, the second biomarker is either LCN15 or OLR1. In various embodiments, the plurality of biomarkers comprise LTBR, LCN15, and OLR1. In various embodiments, a performance of the predictive model is characterized by an area under the curve (AUC) of at least 0.90. In various embodiments, a performance of the predictive model is characterized by a true positive rate of at least 0.8 at a false positive rate of 0.2. In various embodiments, a performance of the predictive model is characterized by a true positive rate of at least 0.9 at a false positive rate of 0.25.

In various embodiments, the plurality of biomarkers comprise LTBP2 and at least a second biomarker. In various embodiments, the plurality of biomarkers comprise TGFA and at least a second biomarker. In various embodiments, the plurality of biomarkers comprise two or more of GDF15, LAMP3, and OSM. In various embodiments, the plurality of biomarkers comprise each of GDF15, LAMP3, and OSM. In various embodiments, a performance of the predictive model is characterized by an area under the curve (AUC) of at least 0.90. In various embodiments, a performance of the predictive model is characterized by a true positive rate of at least 0.8 at a false positive rate of 0.2.

In various embodiments, the plurality of biomarkers comprise two or more of BID, COL4A1, NTF3, PPY, and PRSS22. In various embodiments, the plurality of biomarkers comprise three or more of BID, COL4A1, NTF3, PPY, and PRSS22. In various embodiments, the plurality of biomarkers comprise four or more of BID, COL4A1, NTF3, PPY, and PRSS22. In various embodiments, the plurality of biomarkers comprise each of BID, COL4A1, NTF3, PPY, and PRSS22. In various embodiments, a performance of the predictive model is characterized by an area under the curve (AUC) of at least 0.95. In various embodiments, a performance of the predictive model is characterized by a true positive rate of at least 0.9 at a false positive rate of 0.1.

In various embodiments, the plurality of biomarkers comprise HAVCR2 and OSM. In various embodiments, a performance of the predictive model is characterized by an accuracy of at least 0.85.

In various embodiments, the plurality of biomarkers comprise two or more of CLPS, LTBR, and MMP9. In various embodiments, the plurality of biomarkers comprise each of CLPS, LTBR, and MMP9. In various embodiments, a performance of the predictive model is characterized by an area under the curve (AUC) of at least 0.95. In various embodiments, a performance of the predictive model is characterized by a true positive rate of at least 0.8 at a false positive rate of 0.1.

In various embodiments, the plurality of biomarkers comprise two or more of HEPH, ITGBL1, OSM, and SCARF2. In various embodiments, the plurality of biomarkers comprise three or more of HEPH, ITGBL1, OSM, and SCARF2. In various embodiments, the plurality of biomarkers comprise each of HEPH, ITGBL1, OSM, and SCARF2. In various embodiments, a performance of the predictive model is characterized by an area under the curve (AUC) of at least 0.95. In various embodiments, a performance of the predictive model is characterized by a true positive rate of at least 0.8 at a false positive rate of 0.2. In various embodiments, the plurality of biomarkers comprise ITGBL1 and MMP9. In various embodiments, a performance of the predictive model is characterized by an area under the curve (AUC) of at least 0.90. In various embodiments, a performance of the predictive model is characterized by a true positive rate of at least 0.8 at a false positive rate of 0.2.

In various embodiments, the plurality of biomarkers comprise two or more of COL4A1, FGFR4, NTF3, and PPY. In various embodiments, the plurality of biomarkers comprise three or more of COL4A1, FGFR4, NTF3, and PPY. In various embodiments, the plurality of biomarkers comprise each of COL4A1, FGFR4, NTF3, and PPY. In various embodiments, a performance of the predictive model is characterized by an area under the curve (AUC) of at least 0.95. In various embodiments, a performance of the predictive model is characterized by a true positive rate of at least 0.9 at a false positive rate of 0.1.

In various embodiments, the cancer is lung cancer. In various embodiments, the cancer is an early stage cancer. In various embodiments, the cancer is stage I and/or stage II lung cancer. In various embodiments, the expression levels of the plurality of biomarkers is determined from a test sample obtained from the subject. In various embodiments, the test sample is a blood or serum sample. In various embodiments, the subject is suspected of having an early stage cancer. In various embodiments, the subject is not suspected of having an early stage cancer.

In various embodiments, obtaining or having obtained the dataset comprises performing an assay to determine the expression levels of the plurality of biomarkers. In various embodiments, the assay is a Proximity Extension Assay (PEA), a xMAP Multiplex Assay, a single molecule array (SIMOA) assay, mass spectrometry based protein or peptide assay, or an aptamer-based assay. In various embodiments, performing the assay comprises contacting a test sample with a plurality of reagents comprising antibodies. In various embodiments, the antibodies comprise one of monoclonal and polyclonal antibodies. In various embodiments, the antibodies comprise both monoclonal and polyclonal antibodies.

In various embodiments, methods disclosed herein comprise: responsive to generating a prediction of presence of the early stage cancer in the subject, performing a second analysis to predict presence or absence of the early stage cancer in a subject. In various embodiments, the second analysis achieves a higher specificity in comparison to a specificity of the predictive model. In various embodiments, performing the second analysis comprises performing one or more of CT scan, PET scan, or a tissue biopsy.

In various embodiments, disclosed herein is a non-transitory computer readable medium comprising instructions that, when executed by a processor, cause the processor to: obtain a dataset comprising expression levels of a plurality of biomarkers from the subject, wherein the plurality of biomarkers comprise two or more biomarkers of NTF3, C3, OLR1, MMP9, CSF1, OSM, TNFRSF1A, WFDC2, CLEC5A, BHMT2, PLAUR, TGFA, GLI2, MMP8, LTBR, CXCL8, CD14, SHISA5, CD59, NPDC1, CXCL9, CCL23, COL4A1, PGF, GDF15, COL18A1, NCR3LG1, CXCL12, HAVCR2, HIP1R, RBP7, SPINT1, LTBP2, CALB1, RBFOX3, OCLN, GFRA1, FSTL3, EFNA1, BSG, LRG1, RELT, FGA, ITIH3, TIMP1, TNFRSF1B, CEACAM8, MAMDC2, IL6, FOLR1, CEACAM5, SPP1, CAPG, LGALS9, NPC2, IFI30, ELN, MMP12, VSIG4, NECTIN2, MAD1L1, EDA2R, TNFRSF10B, SMNDC1, PRSS8, CXCL17, PTPRF, TNFRSF10A, CSTB, TREM2, SDC1, DSC2, NME1, LMNB2, CKAP4, EPHB4, LAYN, DLL1, PRG2, SEZ6L2, COLEC12, ULBP2, B4GALT1, HAGH, LCN2, ATRAID, IL1RN, YAP1, TNFSF13, CST3, TNFRSF4, CCL18, POLR2F, EPHA2, SIRPB1, GM2A, SNRPB2, ITIH4, FBLN2, TNFRSF9, CDH2, IL18BP, CWC15, EFNA4, GFAP, ADAMTS16, CHGB, AREG, CCL14, CEACAM6, RNASE1, SPINK1, CD302, KLK7, NRP2, ITGBL1, PRTN3, AGRN, RCC1, THBS2, CRELD1, EFEMP1, SCARB2, C9, CHCHD10, EFHD1, FGL1, IL10RB, KLK4, SEPTIN8, TFF3, CRLF1, COL6A3, CPOX, ADAM8, C4BPB, CXCL16, LAIR1, SCARF2, SERPINB8, IL4R, CD276, CDH23, ANGPT2, ACVRL1, CTSV, GALNT5, RANBP2, VASN, VWA1, RNASE6, APOA2, ICAM1, IL2RA, ZBTB17, OSMR, GRPEL1, IGFBP4, VCAM1, AZU1, CTSD, RNASET2, CD93, SUSD5, SLAMF8, CCL26, IGFBP2, RNF149, MERTK, S100A11, SNED1, CEACAM21, UHRF2, CNDP1, NECTIN4, PIGR, SPRED2, VIPR1, FUT3_FUT5, S100A12, TNFRSF11B, IFNGR1, NPM1, ACTA2, KRT19, SIGLEC5, LAMP3, ALCAM, CD74, PRRT3, ITGA5, TGOLN2, CDCP1, CKB, S100P, SERPINA11, PILRA, NXA1, SLC4A1, NCF2, PTX3, LSP1, CD300A, CLEC7A, LPCAT2, NRP1, CHCHD6, SERPINA3, TNFRSF21, CTSC, LILRB4, NBN, CD55, B2M, ARG1, NGFR, PSMD1, SRP14, ITGB6, AMPD3, CD300E, PKD2, STC2, GCHFR, PGLYRP1, PILRB, CDH3, NMRK2, SMAD1, DCBLD2, CRIM1, HS6ST2, TNFRSF8, CYP24A1, BID, GLRX, TNFRSF14, DPEP2, F9, PTGDS, C2, ERMAP, IGFBPL1, CST1, ELOA, MUC13, IL1R1, S100A3, PIK3IP1, VNN2, TPMT, ANGPTL3, ASGR1, BMP4, CLEC4D, HSPG2, CCL3, CD300LF, COL28A1, CXCL10, QPCT, TGFBR2, COL24A1, CDH6, CD300C, FST, MYBPC2, KCTD5, CSF3, EBI3_IL27, SLC39A14, IL7, CA1, TOR1AIP1, CHI3L1, DGCR6, TNC, CLEC4G, CLPS, ENO3, EPN1, PTPRN2, ADM, LTA4H, TCOF1, TIMD4, CCL28, KLK11, KLK6, LYVE1, TGM2, FRZB, ADAM9, AHSP, CCL2, EGLN1, MRC1, MTUS1, RPS10, TACSTD2, SAA4, SLITRK6, CIT, TNFRSF19, IMMT, ORM1, CTHRC1, KIAA0319, BTN2A1, A1BG, DRAXIN, FGF6, SEMA3F, STC1, BCAM, BAP18, CCL16, DKK3, PODXL2, VWF, FAM20A, DENR, IGFBP7, MSTN, ENOPH1, TSPAN1, EFCAB14, AMBP, C1RL, IL5, TNFSF14, HAVCR1, TNFRSF12A, COL3A1, GPKOW, MANSC1, SEL1L, POSTN, GIPC2, DAPP1, DCN, FAS, GLYR1, LCN15, NEFL, USP28, CHAD, CRH, PBLD, PCNA, CSF2, PBK, BDNF, ROR1, FCN1, ANGPTL4, ZNRD2, CX3CL1, MYH7B, NADK, RAB44, TNFRSF11A, TNFRSF6B, CLMP, HDAC8, IGSF8, PALM2, RECK, CLEC14A, FKBP1B, IL13RA1, WNT9A, C2CD2L, CCDC80, PLA2G2A, SART1, KIRREL2, CCL4, IL18R1, NEO1, FLRT2, TFPI2, LBR, ISLR2, LECT2, PPY, SERPINA1, VWC2, FAM3C, HMBS, LMNB1, PRSS22, CALCB, CCL7, CTSL, FOLR2, PSAP, SEMA7A, GALNT7, NT5C1A, FGFR4, MICB_MICA, BLVRB, BPIFB2, CCN3, GPRC5C, INPP5J, FGFR2, CD83, SCRG1, ALDH3A1, CYTL1, OSCAR, PHLDB1, TNFSF11, GHRL, RRM2, ADGRG1, AXL, CA14, CFH, IL6R, LGALS3, SPON2, CAPS, DCTPP1, MSR1, RARRES2, SCN3A, SORCS2, SCG2, CRYBB2, DNAJA4, LILRA5, REN, COCH, CLEC11A, CRHBP, FARSA, NPHS1, PRAME, PRDX2, CXCL13, ASGR2, BRK1, SCPEP1, GNAS, BCL2L15, C9orf40, CD101, CGB3_CGB5_CGB8, CTSZ, ESM1, CDH17, C5, PON1, OLFM4, OPTC, PALM, PNLIPRP1, PXN, SYNGAP1, MSMB, HEPH, NGRN, CGREF1, LILRB2, NRN1, BCAT2, HNRNPUL1, INSL4, MPO, and PPL; and generate a prediction of presence or absence of the cancer in the subject by applying a predictive model to the expression levels of the plurality of biomarkers.

In various embodiments, a performance of the predictive model is characterized by an area under the curve (AUC) of at least 0.75. In various embodiments, a performance of the predictive model is characterized by an area under the curve (AUC) of at least 0.80. In various embodiments, a performance of the predictive model is characterized by an area under the curve (AUC) of at least 0.85. In various embodiments, a performance of the predictive model is characterized by an area under the curve (AUC) of at least 0.86. In various embodiments, a performance metric of the predictive model is improved in comparison to a model solely incorporating CEA.

In various embodiments, the plurality of biomarkers comprise LTBR and at least a second biomarker. In various embodiments, the second biomarker is either LCN15 or OLR1. In various embodiments, the plurality of biomarkers comprise LTBR, LCN15, and OLR1. In various embodiments, a performance of the predictive model is characterized by an area under the curve (AUC) of at least 0.90. In various embodiments, a performance of the predictive model is characterized by a true positive rate of at least 0.8 at a false positive rate of 0.2. In various embodiments, a performance of the predictive model is characterized by a true positive rate of at least 0.9 at a false positive rate of 0.25.

In various embodiments, the plurality of biomarkers comprise LTBP2 and at least a second biomarker. In various embodiments, the plurality of biomarkers comprise TGFA and at least a second biomarker. In various embodiments, the plurality of biomarkers comprise two or more of GDF15, LAMP3, and OSM. In various embodiments, the plurality of biomarkers comprise each of GDF15, LAMP3, and OSM. In various embodiments, a performance of the predictive model is characterized by an area under the curve (AUC) of at least 0.90. In various embodiments, a performance of the predictive model is characterized by a true positive rate of at least 0.8 at a false positive rate of 0.2.

In various embodiments, the plurality of biomarkers comprise two or more of BID, COL4A1, NTF3, PPY, and PRSS22. In various embodiments, the plurality of biomarkers comprise three or more of BID, COL4A1, NTF3, PPY, and PRSS22. In various embodiments, the plurality of biomarkers comprise four or more of BID, COL4A1, NTF3, PPY, and PRSS22. In various embodiments, the plurality of biomarkers comprise each of BID, COL4A1, NTF3, PPY, and PRSS22. In various embodiments, a performance of the predictive model is characterized by an area under the curve (AUC) of at least 0.95. In various embodiments, a performance of the predictive model is characterized by a true positive rate of at least 0.9 at a false positive rate of 0.1.

In various embodiments, the plurality of biomarkers comprise HAVCR2 and OSM. In various embodiments, a performance of the predictive model is characterized by an accuracy of at least 0.85.

In various embodiments, the plurality of biomarkers comprise two or more of CLPS, LTBR, and MMP9. In various embodiments, the plurality of biomarkers comprise each of CLPS, LTBR, and MMP9. In various embodiments, a performance of the predictive model is characterized by an area under the curve (AUC) of at least 0.95. In various embodiments, a performance of the predictive model is characterized by a true positive rate of at least 0.8 at a false positive rate of 0.1.

In various embodiments, the plurality of biomarkers comprise two or more of HEPH, ITGBL1, OSM, and SCARF2. In various embodiments, the plurality of biomarkers comprise three or more of HEPH, ITGBL1, OSM, and SCARF2. In various embodiments, the plurality of biomarkers comprise each of HEPH, ITGBL1, OSM, and SCARF2. In various embodiments, a performance of the predictive model is characterized by an area under the curve (AUC) of at least 0.95. In various embodiments, a performance of the predictive model is characterized by a true positive rate of at least 0.8 at a false positive rate of 0.2. In various embodiments, the plurality of biomarkers comprise ITGBL1 and MMP9. In various embodiments, a performance of the predictive model is characterized by an area under the curve (AUC) of at least 0.90. In various embodiments, a performance of the predictive model is characterized by a true positive rate of at least 0.8 at a false positive rate of 0.2.

In various embodiments, the plurality of biomarkers comprise two or more of COL4A1, FGFR4, NTF3, and PPY. In various embodiments, the plurality of biomarkers comprise three or more of COL4A1, FGFR4, NTF3, and PPY. In various embodiments, the plurality of biomarkers comprise each of COL4A1, FGFR4, NTF3, and PPY. In various embodiments, a performance of the predictive model is characterized by an area under the curve (AUC) of at least 0.95. In various embodiments, a performance of the predictive model is characterized by a true positive rate of at least 0.9 at a false positive rate of 0.1.

In various embodiments, the cancer is lung cancer. In various embodiments, the cancer is an early stage cancer. In various embodiments, the cancer is stage I and/or stage II lung cancer. In various embodiments, the expression levels of the plurality of biomarkers are determined from a test sample obtained from the subject. In various embodiments, the test sample is a blood or serum sample. In various embodiments, the subject is suspected of having an early stage cancer. In various embodiments, the subject is not suspected of having an early stage cancer.

In various embodiments, non-transitory computer readable media disclosed herein further comprise instructions that, when executed by a processor, cause the processor to: responsive to the generation of a prediction of presence of the early stage cancer in the subject, perform a second analysis to predict presence or absence of the early stage cancer in a subject. In various embodiments, the second analysis achieves a higher specificity in comparison to a specificity of the predictive model.

In various embodiments, disclosed herein is a system comprising: a set of reagents used for determining expression levels for a plurality of biomarkers from a test sample from the subject, wherein the plurality of biomarkers comprise two or more biomarkers of NTF3, C3, OLR1, MMP9, CSF1, OSM, TNFRSF1A, WFDC2, CLEC5A, BHMT2, PLAUR, TGFA, GLI2, MMP8, LTBR, CXCL8, CD14, SHISA5, CD59, NPDC1, CXCL9, CCL23, COL4A1, PGF, GDF15, COL18A1, NCR3LG1, CXCL12, HAVCR2, HIP1R, RBP7, SPINT1, LTBP2, CALB1, RBFOX3, OCLN, GFRA1, FSTL3, EFNA1, BSG, LRG1, RELT, FGA, ITIH3, TIMP1, TNFRSF1B, CEACAM8, MAMDC2, IL6, FOLR1, CEACAM5, SPP1, CAPG, LGALS9, NPC2, IFI30, ELN, MMP12, VSIG4, NECTIN2, MAD1L1, EDA2R, TNFRSF10B, SMNDC1, PRSS8, CXCL17, PTPRF, TNFRSF10A, CSTB, TREM2, SDC1, DSC2, NME1, LMNB2, CKAP4, EPHB4, LAYN, DLL1, PRG2, SEZ6L2, COLEC12, ULBP2, B4GALT1, HAGH, LCN2, ATRAID, IL1RN, YAP1, TNFSF13, CST3, TNFRSF4, CCL18, POLR2F, EPHA2, SIRPB1, GM2A, SNRPB2, ITIH4, FBLN2, TNFRSF9, CDH2, IL18BP, CWC15, EFNA4, GFAP, ADAMTS16, CHGB, AREG, CCL14, CEACAM6, RNASE1, SPINK1, CD302, KLK7, NRP2, ITGBL1, PRTN3, AGRN, RCC1, THBS2, CRELD1, EFEMP1, SCARB2, C9, CHCHD10, EFHD1, FGL1, IL10RB, KLK4, SEPTIN8, TFF3, CRLF1, COL6A3, CPOX, ADAM8, C4BPB, CXCL16, LAIR1, SCARF2, SERPINB8, IL4R, CD276, CDH23, ANGPT2, ACVRL1, CTSV, GALNT5, RANBP2, VASN, VWA1, RNASE6, APOA2, ICAM1, IL2RA, ZBTB17, OSMR, GRPEL1, IGFBP4, VCAM1, AZU1, CTSD, RNASET2, CD93, SUSD5, SLAMF8, CCL26, IGFBP2, RNF149, MERTK, S100A11, SNED1, CEACAM21, UHRF2, CNDP1, NECTIN4, PIGR, SPRED2, VIPR1, FUT3_FUT5, S100A12, TNFRSF11B, IFNGR1, NPM1, ACTA2, KRT19, SIGLEC5, LAMP3, ALCAM, CD74, PRRT3, ITGA5, TGOLN2, CDCP1, CKB, S100P, SERPINA11, PILRA, NXA1, SLC4A1, NCF2, PTX3, LSP1, CD300A, CLEC7A, LPCAT2, NRP1, CHCHD6, SERPINA3, TNFRSF21, CTSC, LILRB4, NBN, CD55, B2M, ARG1, NGFR, PSMD1, SRP14, ITGB6, AMPD3, CD300E, PKD2, STC2, GCHFR, PGLYRP1, PILRB, CDH3, NMRK2, SMAD1, DCBLD2, CRIM1, HS6ST2, TNFRSF8, CYP24A1, BID, GLRX, TNFRSF14, DPEP2, F9, PTGDS, C2, ERMAP, IGFBPL1, CST1, ELOA, MUC13, IL1R1, S100A3, PIK3IP1, VNN2, TPMT, ANGPTL3, ASGR1, BMP4, CLEC4D, HSPG2, CCL3, CD300LF, COL28A1, CXCL10, QPCT, TGFBR2, COL24A1, CDH6, CD300C, FST, MYBPC2, KCTD5, CSF3, EBI3_IL27, SLC39A14, IL7, CA1, TOR1AIP1, CHI3L1, DGCR6, TNC, CLEC4G, CLPS, ENO3, EPN1, PTPRN2, ADM, LTA4H, TCOF1, TIMD4, CCL28, KLK11, KLK6, LYVE1, TGM2, FRZB, ADAM9, AHSP, CCL2, EGLN1, MRC1, MTUS1, RPS10, TACSTD2, SAA4, SLITRK6, CIT, TNFRSF19, IMMT, ORM1, CTHRC1, KIAA0319, BTN2A1, A1BG, DRAXIN, FGF6, SEMA3F, STC1, BCAM, BAP18, CCL16, DKK3, PODXL2, VWF, FAM20A, DENR, IGFBP7, MSTN, ENOPH1, TSPAN1, EFCAB14, AMBP, C1RL, IL5, TNFSF14, HAVCR1, TNFRSF12A, COL3A1, GPKOW, MANSC1, SEL1L, POSTN, GIPC2, DAPP1, DCN, FAS, GLYR1, LCN15, NEFL, USP28, CHAD, CRH, PBLD, PCNA, CSF2, PBK, BDNF, ROR1, FCN1, ANGPTL4, ZNRD2, CX3CL1, MYH7B, NADK, RAB44, TNFRSF11A, TNFRSF6B, CLMP, HDAC8, IGSF8, PALM2, RECK, CLEC14A, FKBP1B, IL13RA1, WNT9A, C2CD2L, CCDC80, PLA2G2A, SART1, KIRREL2, CCL4, IL18R1, NEO1, FLRT2, TFPI2, LBR, ISLR2, LECT2, PPY, SERPINA1, VWC2, FAM3C, HMBS, LMNB1, PRSS22, CALCB, CCL7, CTSL, FOLR2, PSAP, SEMA7A, GALNT7, NT5C1A, FGFR4, MICB_MICA, BLVRB, BPIFB2, CCN3, GPRC5C, INPP5J, FGFR2, CD83, SCRG1, ALDH3A1, CYTL1, OSCAR, PHLDB1, TNFSF11, GHRL, RRM2, ADGRG1, AXL, CA14, CFH, IL6R, LGALS3, SPON2, CAPS, DCTPP1, MSR1, RARRES2, SCN3A, SORCS2, SCG2, CRYBB2, DNAJA4, LILRA5, REN, COCH, CLEC11A, CRHBP, FARSA, NPHS1, PRAME, PRDX2, CXCL13, ASGR2, BRK1, SCPEP1, GNAS, BCL2L15, C9orf40, CD101, CGB3 CGB5 CGB8, CTSZ, ESM1, CDH17, C5, PON1, OLFM4, OPTC, PALM, PNLIPRP1, PXN, SYNGAP1, MSMB, HEPH, NGRN, CGREF1, LILRB2, NRN1, BCAT2, HNRNPUL1, INSL4, MPO, and PPL; an apparatus configured to receive a mixture of one or more reagents in the set and the test sample and to measure the expression levels for the biomarkers from the test sample; and a computer system communicatively coupled to the apparatus to obtain a dataset comprising the expression levels for the plurality of biomarkers from the test sample and to generate a presence or absence of cancer in the subject by applying a predictive model to the expression levels of the plurality of biomarkers.

In various embodiments, a performance of the predictive model is characterized by an area under the curve (AUC) of at least 0.75. In various embodiments, a performance of the predictive model is characterized by an area under the curve (AUC) of at least 0.80.

In various embodiments, a performance of the predictive model is characterized by an area under the curve (AUC) of at least 0.75. In various embodiments, a performance of the predictive model is characterized by an area under the curve (AUC) of at least 0.80. In various embodiments, a performance of the predictive model is characterized by an area under the curve (AUC) of at least 0.85. In various embodiments, a performance of the predictive model is characterized by an area under the curve (AUC) of at least 0.86. In various embodiments, a performance metric of the predictive model is improved in comparison to a model solely incorporating CEA.

In various embodiments, the plurality of biomarkers comprise LTBR and at least a second biomarker. In various embodiments, the second biomarker is either LCN15 or OLR1. In various embodiments, the plurality of biomarkers comprise LTBR, LCN15, and OLR1. In various embodiments, a performance of the predictive model is characterized by an area under the curve (AUC) of at least 0.90. In various embodiments, a performance of the predictive model is characterized by a true positive rate of at least 0.8 at a false positive rate of 0.2. In various embodiments, a performance of the predictive model is characterized by a true positive rate of at least 0.9 at a false positive rate of 0.25.

In various embodiments, the plurality of biomarkers comprise LTBP2 and at least a second biomarker. In various embodiments, the plurality of biomarkers comprise TGFA and at least a second biomarker. In various embodiments, the plurality of biomarkers comprise two or more of GDF15, LAMP3, and OSM. In various embodiments, the plurality of biomarkers comprise each of GDF15, LAMP3, and OSM. In various embodiments, a performance of the predictive model is characterized by an area under the curve (AUC) of at least 0.90. In various embodiments, a performance of the predictive model is characterized by a true positive rate of at least 0.8 at a false positive rate of 0.2.

In various embodiments, the plurality of biomarkers comprise two or more of BID, COL4A1, NTF3, PPY, and PRSS22. In various embodiments, the plurality of biomarkers comprise three or more of BID, COL4A1, NTF3, PPY, and PRSS22. In various embodiments, the plurality of biomarkers comprise four or more of BID, COL4A1, NTF3, PPY, and PRSS22. In various embodiments, the plurality of biomarkers comprise each of BID, COL4A1, NTF3, PPY, and PRSS22. In various embodiments, a performance of the predictive model is characterized by an area under the curve (AUC) of at least 0.95. In various embodiments, a performance of the predictive model is characterized by a true positive rate of at least 0.9 at a false positive rate of 0.1.

In various embodiments, the plurality of biomarkers comprise HAVCR2 and OSM. In various embodiments, a performance of the predictive model is characterized by an accuracy of at least 0.85.

In various embodiments, the plurality of biomarkers comprise two or more of CLPS, LTBR, and MMP9. In various embodiments, the plurality of biomarkers comprise each of CLPS, LTBR, and MMP9. In various embodiments, a performance of the predictive model is characterized by an area under the curve (AUC) of at least 0.95. In various embodiments, a performance of the predictive model is characterized by a true positive rate of at least 0.8 at a false positive rate of 0.1.

In various embodiments, the plurality of biomarkers comprise two or more of HEPH, ITGBL1, OSM, and SCARF2. In various embodiments, the plurality of biomarkers comprise three or more of HEPH, ITGBL1, OSM, and SCARF2. In various embodiments, the plurality of biomarkers comprise each of HEPH, ITGBL1, OSM, and SCARF2. In various embodiments, a performance of the predictive model is characterized by an area under the curve (AUC) of at least 0.95. In various embodiments, a performance of the predictive model is characterized by a true positive rate of at least 0.8 at a false positive rate of 0.2. In various embodiments, the plurality of biomarkers comprise ITGBL1 and MMP9. In various embodiments, a performance of the predictive model is characterized by an area under the curve (AUC) of at least 0.90. In various embodiments, a performance of the predictive model is characterized by a true positive rate of at least 0.8 at a false positive rate of 0.2.

In various embodiments, the plurality of biomarkers comprise two or more of COL4A1, FGFR4, NTF3, and PPY. In various embodiments, the plurality of biomarkers comprise three or more of COL4A1, FGFR4, NTF3, and PPY. In various embodiments, the plurality of biomarkers comprise each of COL4A1, FGFR4, NTF3, and PPY. In various embodiments, a performance of the predictive model is characterized by an area under the curve (AUC) of at least 0.95. In various embodiments, a performance of the predictive model is characterized by a true positive rate of at least 0.9 at a false positive rate of 0.1.

In various embodiments, the cancer is lung cancer. In various embodiments, the cancer is an early stage cancer. In various embodiments, the cancer is stage I and/or stage II lung cancer. In various embodiments, the expression levels of the plurality of biomarkers are determined from a test sample obtained from the subject. In various embodiments, the test sample is a blood or serum sample. In various embodiments, the subject is suspected of having an early stage cancer. In various embodiments, the subject is not suspected of having an early stage cancer.

In various embodiments, the computer system is further configured to: responsive to the generation of a prediction of presence of the early stage cancer in the subject, perform a second analysis to predict presence or absence of the early stage cancer in a subject. In various embodiments, the second analysis achieves a higher specificity in comparison to a specificity of the predictive model.

In various embodiments, disclosed herein is a kit for predicting presence or absence of cancer in a subject, the kit comprising: a set of reagents for determining expression levels for a plurality of biomarkers from a test sample from the subject, wherein the plurality of biomarkers comprise two or more biomarkers of NTF3, C3, OLR1, MMP9, CSF1, OSM, TNFRSF1A, WFDC2, CLEC5A, BHMT2, PLAUR, TGFA, GLI2, MMP8, LTBR, CXCL8, CD14, SHISA5, CD59, NPDC1, CXCL9, CCL23, COL4A1, PGF, GDF15, COL18A1, NCR3LG1, CXCL12, HAVCR2, HIP1R, RBP7, SPINT1, LTBP2, CALB1, RBFOX3, OCLN, GFRA1, FSTL3, EFNA1, BSG, LRG1, RELT, FGA, ITIH3, TIMP1, TNFRSF1B, CEACAM8, MAMDC2, IL6, FOLR1, CEACAM5, SPP1, CAPG, LGALS9, NPC2, IF130, ELN, MMP12, VSIG4, NECTIN2, MAD1L1, EDA2R, TNFRSF10B, SMNDC1, PRSS8, CXCL17, PTPRF, TNFRSF10A, CSTB, TREM2, SDC1, DSC2, NME1, LMNB2, CKAP4, EPHB4, LAYN, DLL1, PRG2, SEZ6L2, COLEC12, ULBP2, B4GALT1, HAGH, LCN2, ATRAID, IL1RN, YAP1, TNFSF13, CST3, TNFRSF4, CCL18, POLR2F, EPHA2, SIRPB1, GM2A, SNRPB2, ITIH4, FBLN2, TNFRSF9, CDH2, IL18BP, CWC15, EFNA4, GFAP, ADAMTS16, CHGB, AREG, CCL14, CEACAM6, RNASE1, SPINK1, CD302, KLK7, NRP2, ITGBL1, PRTN3, AGRN, RCC1, THBS2, CRELD1, EFEMP1, SCARB2, C9, CHCHD10, EFHD1, FGL1, IL10RB, KLK4, SEPTIN8, TFF3, CRLF1, COL6A3, CPOX, ADAM8, C4BPB, CXCL16, LAIR1, SCARF2, SERPINB8, IL4R, CD276, CDH23, ANGPT2, ACVRL1, CTSV, GALNT5, RANBP2, VASN, VWA1, RNASE6, APOA2, ICAM1, IL2RA, ZBTB17, OSMR, GRPEL1, IGFBP4, VCAM1, AZU1, CTSD, RNASET2, CD93, SUSD5, SLAMF8, CCL26, IGFBP2, RNF149, MERTK, S100A11, SNED1, CEACAM21, UHRF2, CNDP1, NECTIN4, PIGR, SPRED2, VIPR1, FUT3_FUT5, S100A12, TNFRSF11B, IFNGR1, NPM1, ACTA2, KRT19, SIGLEC5, LAMP3, ALCAM, CD74, PRRT3, ITGA5, TGOLN2, CDCP1, CKB, S100P, SERPINA11, PILRA, NXA1, SLC4A1, NCF2, PTX3, LSP1, CD300A, CLEC7A, LPCAT2, NRP1, CHCHD6, SERPINA3, TNFRSF21, CTSC, LILRB4, NBN, CD55, B2M, ARG1, NGFR, PSMD1, SRP14, ITGB6, AMPD3, CD300E, PKD2, STC2, GCHFR, PGLYRP1, PILRB, CDH3, NMRK2, SMAD1, DCBLD2, CRIM1, HS6ST2, TNFRSF8, CYP24A1, BID, GLRX, TNFRSF14, DPEP2, F9, PTGDS, C2, ERMAP, IGFBPL1, CST1, ELOA, MUC13, IL1R1, S100A3, PIK3IP1, VNN2, TPMT, ANGPTL3, ASGR1, BMP4, CLEC4D, HSPG2, CCL3, CD300LF, COL28A1, CXCL10, QPCT, TGFBR2, COL24A1, CDH6, CD300C, FST, MYBPC2, KCTD5, CSF3, EBI3_IL27, SLC39A14, IL7, CA1, TOR1AIP1, CHI3L1, DGCR6, TNC, CLEC4G, CLPS, ENO3, EPN1, PTPRN2, ADM, LTA4H, TCOF1, TIMD4, CCL28, KLK11, KLK6, LYVE1, TGM2, FRZB, ADAM9, AHSP, CCL2, EGLN1, MRC1, MTUS1, RPS10, TACSTD2, SAA4, SLITRK6, CIT, TNFRSF19, IMMT, ORM1, CTHRC1, KIAA0319, BTN2A1, A1BG, DRAXIN, FGF6, SEMA3F, STC1, BCAM, BAP18, CCL16, DKK3, PODXL2, VWF, FAM20A, DENR, IGFBP7, MSTN, ENOPH1, TSPAN1, EFCAB14, AMBP, C1RL, IL5, TNFSF14, HAVCR1, TNFRSF12A, COL3A1, GPKOW, MANSC1, SEL1L, POSTN, GIPC2, DAPP1, DCN, FAS, GLYR1, LCN15, NEFL, USP28, CHAD, CRH, PBLD, PCNA, CSF2, PBK, BDNF, ROR1, FCN1, ANGPTL4, ZNRD2, CX3CL1, MYH7B, NADK, RAB44, TNFRSF11A, TNFRSF6B, CLMP, HDAC8, IGSF8, PALM2, RECK, CLEC14A, FKBP1B, IL13RA1, WNT9A, C2CD2L, CCDC80, PLA2G2A, SART1, KIRREL2, CCL4, IL18R1, NEO1, FLRT2, TFPI2, LBR, ISLR2, LECT2, PPY, SERPINA1, VWC2, FAM3C, HMBS, LMNB1, PRSS22, CALCB, CCL7, CTSL, FOLR2, PSAP, SEMA7A, GALNT7, NT5C1A, FGFR4, MICB_MICA, BLVRB, BPIFB2, CCN3, GPRC5C, INPP5J, FGFR2, CD83, SCRG1, ALDH3A1, CYTL1, OSCAR, PHLDB1, TNFSF11, GHRL, RRM2, ADGRG1, AXL, CA14, CFH, IL6R, LGALS3, SPON2, CAPS, DCTPP1, MSR1, RARRES2, SCN3A, SORCS2, SCG2, CRYBB2, DNAJA4, LILRA5, REN, COCH, CLEC11A, CRHBP, FARSA, NPHS1, PRAME, PRDX2, CXCL13, ASGR2, BRK1, SCPEP1, GNAS, BCL2L15, C9orf40, CD101, CGB3_CGB5_CGB8, CTSZ, ESM1, CDH17, C5, PON1, OLFM4, OPTC, PALM, PNLIPRP1, PXN, SYNGAP1, MSMB, HEPH, NGRN, CGREF1, LILRB2, NRN1, BCAT2, HNRNPUL1, INSL4, MPO, and PPL; and instructions for using the set of reagents to determine the expression levels of the plurality of biomarkers from the test sample and to generate a prediction of presence or absence of cancer in the subject by applying a predictive model to the expression levels of the plurality of biomarkers.

In various embodiments, a performance of the predictive model is characterized by an area under the curve (AUC) of at least 0.75. In various embodiments, a performance of the predictive model is characterized by an area under the curve (AUC) of at least 0.80. In various embodiments, a performance of the predictive model is characterized by an area under the curve (AUC) of at least 0.85. In various embodiments, a performance of the predictive model is characterized by an area under the curve (AUC) of at least 0.86. In various embodiments, a performance metric of the predictive model is improved in comparison to a model solely incorporating CEA.

In various embodiments, the plurality of biomarkers comprise LTBR and at least a second biomarker. In various embodiments, the second biomarker is either LCN15 or OLR1. In various embodiments, the plurality of biomarkers comprise LTBR, LCN15, and OLR1. In various embodiments, a performance of the predictive model is characterized by an area under the curve (AUC) of at least 0.90. In various embodiments, a performance of the predictive model is characterized by a true positive rate of at least 0.8 at a false positive rate of 0.2. In various embodiments, a performance of the predictive model is characterized by a true positive rate of at least 0.9 at a false positive rate of 0.25.

In various embodiments, the plurality of biomarkers comprise LTBP2 and at least a second biomarker. In various embodiments, the plurality of biomarkers comprise TGFA and at least a second biomarker. In various embodiments, the plurality of biomarkers comprise two or more of GDF15, LAMP3, and OSM. In various embodiments, the plurality of biomarkers comprise each of GDF15, LAMP3, and OSM. In various embodiments, a performance of the predictive model is characterized by an area under the curve (AUC) of at least 0.90. In various embodiments, a performance of the predictive model is characterized by a true positive rate of at least 0.8 at a false positive rate of 0.2.

In various embodiments, the plurality of biomarkers comprise two or more of BID, COL4A1, NTF3, PPY, and PRSS22. In various embodiments, the plurality of biomarkers comprise three or more of BID, COL4A1, NTF3, PPY, and PRSS22. In various embodiments, the plurality of biomarkers comprise four or more of BID, COL4A1, NTF3, PPY, and PRSS22. In various embodiments, the plurality of biomarkers comprise each of BID, COL4A1, NTF3, PPY, and PRSS22. In various embodiments, a performance of the predictive model is characterized by an area under the curve (AUC) of at least 0.95. In various embodiments, a performance of the predictive model is characterized by a true positive rate of at least 0.9 at a false positive rate of 0.1.

In various embodiments, the plurality of biomarkers comprise HAVCR2 and OSM. In various embodiments, a performance of the predictive model is characterized by an accuracy of at least 0.85.

In various embodiments, the plurality of biomarkers comprise two or more of CLPS, LTBR, and MMP9. In various embodiments, the plurality of biomarkers comprise each of CLPS, LTBR, and MMP9. In various embodiments, a performance of the predictive model is characterized by an area under the curve (AUC) of at least 0.95. In various embodiments, a performance of the predictive model is characterized by a true positive rate of at least 0.8 at a false positive rate of 0.1.

In various embodiments, the plurality of biomarkers comprise two or more of HEPH, ITGBL1, OSM, and SCARF2. In various embodiments, the plurality of biomarkers comprise three or more of HEPH, ITGBL1, OSM, and SCARF2. In various embodiments, the plurality of biomarkers comprise each of HEPH, ITGBL1, OSM, and SCARF2. In various embodiments, a performance of the predictive model is characterized by an area under the curve (AUC) of at least 0.95. In various embodiments, a performance of the predictive model is characterized by a true positive rate of at least 0.8 at a false positive rate of 0.2. In various embodiments, the plurality of biomarkers comprise ITGBL1 and MMP9. In various embodiments, a performance of the predictive model is characterized by an area under the curve (AUC) of at least 0.90. In various embodiments, a performance of the predictive model is characterized by a true positive rate of at least 0.8 at a false positive rate of 0.2.

In various embodiments, the plurality of biomarkers comprise two or more of COL4A1, FGFR4, NTF3, and PPY. In various embodiments, the plurality of biomarkers comprise three or more of COL4A1, FGFR4, NTF3, and PPY. In various embodiments, the plurality of biomarkers comprise each of COL4A1, FGFR4, NTF3, and PPY. In various embodiments, a performance of the predictive model is characterized by an area under the curve (AUC) of at least 0.95. In various embodiments, a performance of the predictive model is characterized by a true positive rate of at least 0.9 at a false positive rate of 0.1. In various embodiments, the cancer is lung cancer. In various embodiments, the cancer is an early stage cancer. In various embodiments, the cancer is stage I and/or stage II lung cancer.

In various embodiments, the test sample is a blood or serum sample. In various embodiments, the subject is suspected of having an early stage cancer. In various embodiments, the subject is not suspected of having an early stage cancer. In various embodiments, the set of reagents is used to perform an assay to determine the expression levels of the plurality of biomarkers. In various embodiments, wherein the assay is a Proximity Extension Assay (PEA), a xMAP Multiplex Assay, a single molecule array (SIMOA) assay, mass spectrometry based protein or peptide assay, or an aptamer-based assay. In various embodiments, performing the assay comprises contacting a test sample with a plurality of reagents comprising antibodies. In various embodiments, the antibodies comprise one of monoclonal and polyclonal antibodies. In various embodiments, the antibodies comprise both monoclonal and polyclonal antibodies.

In various embodiments, kits disclosed herein further comprise instructions for performing a second analysis to predict presence or absence of the early stage cancer in a subject. In various embodiments, the second analysis achieves a higher specificity in comparison to a specificity of the predictive model.

### EXAMPLES

Below are examples of specific embodiments for carrying out the present invention. The examples are offered for illustrative purposes only and are not intended to limit the scope of the present invention in any way. Efforts have been made to ensure accuracy with respect to numbers used, but some experimental error and deviation should be allowed for.

### Example 1: Human Clinical Studies and Sample Analysis

Human lung cancer samples and human non-cancer control samples were obtained for analysis of biomarker expression levels. For each subject, a plasma sample was obtained.

Blood samples were collected into Cell Free Blood Collection Tubes (Streck). Plasma and leukocyte fractions were prepared. Plasma was prepared with a single spin protocol, 1600 g for 10 min at room temperature. Plasma was then aliquoted into 2 mL cryovials. One of these aliquots was then provided to Olink® for performing protein biomarker assays (e.g., Proximity Extension Assay (PEA)).

The breakdown of the subjects from whom the samples were obtained is shown in Table 1 (total N, age, and smoking history).

Of the 34 subjects with known cancer, the cancer stage distribution was as follows:
Stage 1: 10 subjects (29%)
Stage 2: 2 subjects (6%)
Stage 3: 12 subjects (35%)
Stage 4: 9 subjects (27%)
Undetermined: 1 subject (3%)

Of the 34 subjects with known cancer, the cancer subtype distribution was as follows:
Adenocarcinoma: 14 subjects (41%)
Squamous: 11 subjects (32%)
Neuroendocrine: 3 subjects (9%)
Small cell lung cancer: 1 subject (3%)
Non-small cell lung cancer: 1 subject (3%)
Large cell: 1 subject (3%)
Adenosquamous: 1 subject (3%)
Undetermined: 2 subjects (3%)

### Example 2: Univariate Analysis

Univariate analyses were conducted to identify potential biomarkers that distinguished cancer samples and non-cancer samples. These potential biomarkers were then considered for inclusion in a multivariate biomarker panel.

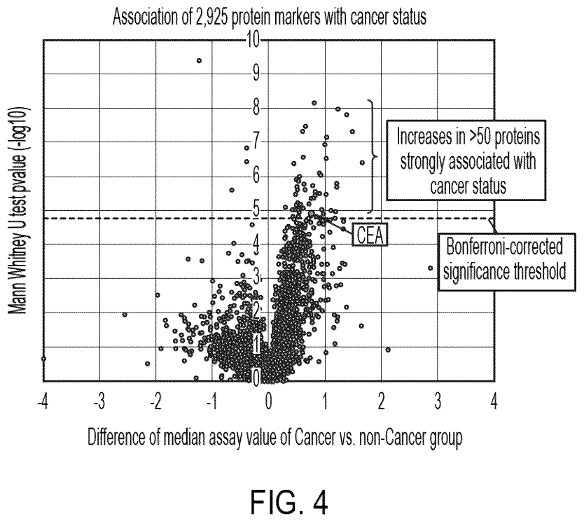

Specifically, for each individual biomarker, the assay value of the biomarker in cancer samples and the assay value of the biomarker in non-cancer samples were determined. For a particular biomarker, the larger the difference between the two sets of assay values, the more likely the biomarker is a strong indicator for lung cancer. Reference is now made to FIG. 4, which shows univariate analyses of individual biomarkers (e.g., 2,925 protein biomarkers) for distinguishing cancer versus non-cancer groups. Here, the x-axis shows the difference of median assay values of the biomarker in cancer samples versus non-cancer samples. The y-axis shows the transformed Mann Whitney test p-value (e.g., expressed as −log(pvalue)). Furthermore, FIG. 4 identifies carcinoembryonic antigen (CEA), which is an established biomarker known to be associated with cancer. Here, FIG. 4 shows the presence of multiple protein biomarkers that are more strongly associated with cancer status in comparison to the known CEA biomarker. Additionally, Table 2 identifies the top 473 protein biomarkers identified via the univariate analyses. Here, the identified 473 biomarkers were included as they satisfied an FDR 5% p-value cut off of 0.008060. The identified 473 biomarkers were further analyzed, as described in the further Examples below.

### Example 3: Biomarker Pair Analysis

Biomarker pairs were analyzed for their ability to predict cancer status. In this example, the paired analysis was conducted on a 355 protein subset of the previously identified 473 protein biomarkers. Here, the biomarkers of the 355 protein subset had positive associations with cancer (Median difference >0 as shown in Table 2) and used dilution level 1:100 or less on the Olink platform (i.e., excluding very high abundance proteins).

For each biomarker pair, a logistic regression model was trained to distinguish between cancer and non-cancerous status based on the expression values of the biomarkers of the biomarker pair. The logistic regression model had the standard form with an intercept term and a parameter for each of the two biomarkers. No interaction term was included. Scikit-learn library was used with the newton-cg solver and no penalty. Logistic regression models underwent evaluation through 5-fold cross-validation.

Top performing biomarker pairs (e.g., with an accuracy above ˜0.75) are shown in Table 4. In total, Table 4 includes 6372 biomarker pairs selected from the 355 protein subset. Altogether, this establishes that two biomarkers (which were individually identified as positively associated with cancer through the univariate analysis described above) can be combined as a panel for predicting lung cancer status.

### Example 4: Additional Biomarker Combination Analysis

Biomarker combinations (e.g., two biomarker combinations, three biomarker combinations, four biomarker combinations, five biomarker combinations, eight biomarker combinations, ten biomarker combinations, fifteen biomarker combinations, and seventeen biomarker combinations) were analyzed for their ability to predict lung cancer status. Biomarker combinations were selected from 17 biomarkers of: TGFA, S100A12, OSM, TFPI2, LSP1, MDK, CXCL9, CLEC4D, IL6, HGF, VWA1, CEACAM5, MMP12, KRT19, CASP8, WFDC2, and PLAUR. These 17 biomarkers had positive associations with cancer (Median difference >0 as shown in Table 3).

Specifically, the 17 biomarkers were identified by analyzing circulating protein level data from 235 of study subjects, including 110 cancer patients and 125 non-cancer controls. In brief, plasma samples were prepared on site and sent for analysis (e.g., to Olink) in 96 well plates. Plasma samples were stored at all times before plating at −80 C. During plating both the thawing of frozen plasma and the plating itself occurred on wet ice. Each sample was plated using 100 μL of plasma and the plated samples were refrozen at −80 C and shipped on dry ice. The Olink Proximity Extension Assay (PEA) was conducted to determine expression levels of various biomarkers, including the 17 biomarkers described above. Further details of the Olink Proximity Extension Assay (PEA) is described in Wik, L., et al. (2021). Proximity Extension Assay in Combination with Next-Generation Sequencing for High-throughput Proteome-wide Analysis. Molecular & cellular proteomics: MCP, 20, 100168, which is hereby incorporated by reference in its entirety

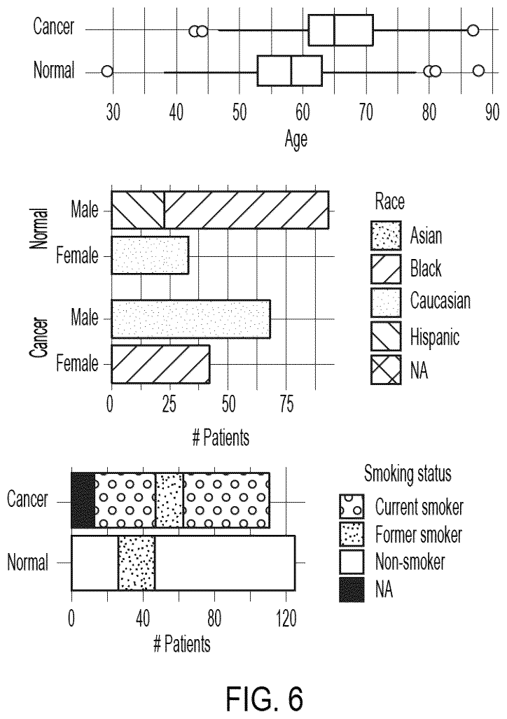

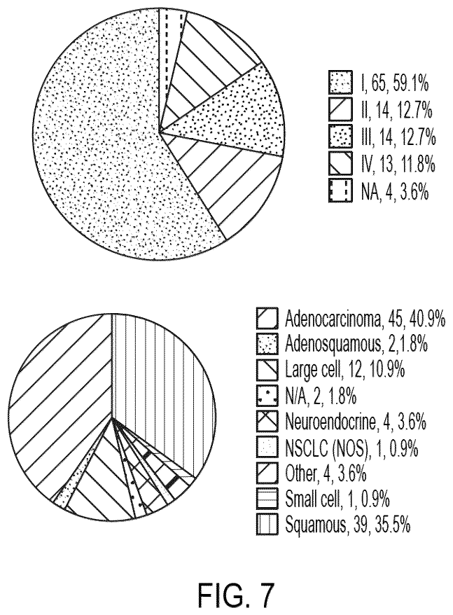

Demographic and tumor properties distribution of these subjects are shown in FIG. 6 and FIG. 7. 18 biomarkers were significantly associated with cancer status in the cohort at FDR<0.05. Notably, 17 of the 18 were positively associated with cancer status. One additional protein (ALPP) was associated with cancer status in the cohort (FDR<0.05) but in the opposite direction.

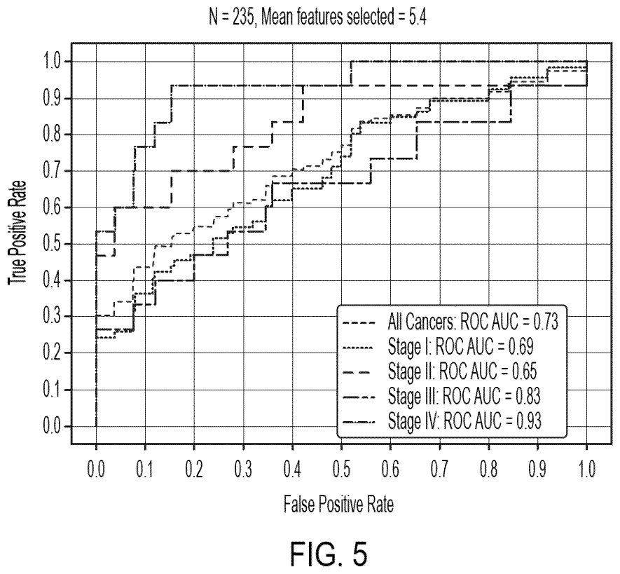

For each biomarker combination, a support vector machine (SVM) classifier, with a radial basis function kernel and regularization parameter C=0.1, was trained to distinguish between cancer and non-cancerous status based on the expression values of the biomarkers of the biomarker combination. Forward feature selection with 5-fold cross-validation resulted in models with an average of approximately 5 features selected, achieving an overall cross-validated ROC AUC of 0.73 across all stages of cancers (FIG. 5). Notably, the models in this example achieved the best performance for late stage cancers (e.g., AUC=0.93 for stage IV cancer and AUC=0.83 for stage III cancer). The models remained predictive for early stage cancers (e.g., AUC=0.69 for stage I cancer and AUC=0.65 for stage II cancer).

Next, performance of all SVM models with a radial basis function kernel and a regularization parameter C=0.1 was evaluated and included between 1 to 5 of the 17 protein markers. All combinations of markers with AUC equal to or greater than 0.6 are shown in Table 5. In total, Table 5 includes 7960 biomarker combinations selected from the 17 protein subset. Altogether, this establishes that combining two or more of these biomarkers (which were individually identified as positively associated with cancer through the univariate analysis described above) represents biomarker panel(s) for predicting lung cancer status.

Lengthy table referenced here
US20250014761A1-20250109-T00001
Please refer to the end of the specification for access instructions.

Lengthy table referenced here
US20250014761A1-20250109-T00002
Please refer to the end of the specification for access instructions.

Lengthy table referenced here
US20250014761A1-20250109-T00003
Please refer to the end of the specification for access instructions.

Lengthy table referenced here
US20250014761A1-20250109-T00004
Please refer to the end of the specification for access instructions.

Lengthy table referenced here
US20250014761A1-20250109-T00005
Please refer to the end of the specification for access instructions.

LENGTHY TABLES
The patent application contains a lengthy table section. A copy of the table is available in electronic form from the USPTO web site (https://seqdata.uspto.gov/?pageRequest=docDetail&DocID=US20250014761A1). An electronic copy of the table will also be available from the USPTO upon request and payment of the fee set forth in 37 CFR 1.19(b)(3).

1. A method for predicting presence or absence of cancer in a subject, the method comprising:
obtaining or having obtained a dataset comprising expression levels of a plurality of biomarkers from the subject, wherein the plurality of biomarkers comprises at two or more biomarkers selected from: IL6, TGFA, S100A12, OSM, TFPI2, LSP1, MDK, CXCL9, CLEC4D, HGF, VWA1, CEACAM5, MMP12, KRT19, CASP8, WFDC2, and PLAUR; and
generating a prediction of presence or absence of the cancer in the subject by applying a predictive model to the expression levels of the plurality of biomarkers.

2. The method of claim 1, wherein a performance of the predictive model is characterized by an area under the curve (AUC) of at least 0.60, at least 0.61, at least 0.62, at least 0.63, at least 0.64, at least 0.65, at least 0.66, at least 0.67, at least 0.68, at least 0.69, at least 0.70, at least 0.71, at least 0.72, at least 0.73, or at least 0.74.

3. The method of any one of claims 1-2, wherein the performance of the predictive model is characterized by an area under the curve (AUC) of at least 0.60.

4. The method of any one of claims 1-3, wherein the performance of the predictive model is characterized by an area under the curve (AUC) of at least 0.74.

5. The method of any one of claims 1-4, wherein a performance metric of the predictive model is improved in comparison to a model solely incorporating CEACAM5.

6. The method of any one of claims 1-5, wherein the predictive model comprises a support vector machine (SVM) classifier.

7. The method of any one of claims 1-6, wherein the plurality of biomarkers comprises IL6 and at least one more biomarker.

8. The method of claim 7, wherein the at least one more biomarker is selected from the group comprising: TGFA, S100A12, OSM, TFPI2, LSP1, MDK, CXCL9, CLEC4D, HGF, VWA1, CEACAM5, MMP12, KRT19, CASP8, WFDC2, and PLAUR.

9. The method of any one of claims 7-8, wherein the plurality of biomarkers is selected from a combination of biomarkers as shown in Table 5.

10. The method of any one of claims 7-9, wherein a performance of the predictive model is characterized by an area under the curve (AUC) of at least 0.60.

11. The method of any one of claims 7-10, wherein a performance of the predictive model is characterized by a true positive rate of at least 30% at a false positive rate of 10%.

12. The method of any one of claims 1-6, wherein the plurality of biomarkers comprises IL6 and at least one more biomarker selected from the group comprising: TGFA, S100A12, OSM, TFPI2, LSP1, MDK, CXCL9, HGF, VWA1, CEACAM5, MMP12, KRT19, CASP8, WFDC2, and PLAUR.

13. The method of claim 12, wherein the plurality of biomarkers is selected from a combination of biomarkers as shown in Table 5.

14. The method of any one of claims 12-13, wherein a performance of the predictive model is characterized by an area under the curve (AUC) of at least 0.72.

15. The method of any one of claims 12-14, wherein a performance of the predictive model is characterized by a true positive rate of at least 30% at a false positive rate of 10%.

16. The method of any one of claims 1-6, wherein the plurality of biomarkers comprises IL6 and at least one more biomarker selected from the group comprising: TGFA, S100A12, OSM, LSP1, MDK, CXCL9, HGF, CEACAM5, MMP12, KRT19, WFDC2, and PLAUR.

17. The method of claim 16, wherein the plurality of biomarkers is selected from the group comprising:
a. IL6, LSP1, MDK, MMP12;
b. CEACAM5, IL6, MDK, MMP12, TGFA;
c. HGF, IL6, MDK, MMP12, TGFA;
d. CEACAM5, IL6, MDK, TGFA;
e. IL6, MDK, MMP12, OSM;
f. IL6, MDK, MMP12, TGFA;
g. CEACAM5, IL6, LSP1, MDK, TGFA;
h. HGF, IL6, MDK, MMP12, OSM;
i. HGF, IL6, LSP1, MDK, MMP12;
j. IL6, KRT19, MDK, MMP12, TGFA;
k. HGF, IL6, LSP1, MDK;
l. IL6, LSP1, MDK;
m. IL6, LSP1, MDK, TGFA;
n. IL6, MDK, TGFA;
o. CXCL9, IL6, LSP1, MDK;
p. CEACAM5, IL6, MDK, OSM, TGFA;
q. CEACAM5, HGF, IL6, MDK, TGFA;
r. CEACAM5, IL6, MDK, OSM;
s. CEACAM5, IL6, MDK, MMP12, OSM;
t. HGF, IL6, LSP1, MDK, TGFA;
u. CEACAM5, IL6, LSP1, MDK;
v. CEACAM5, IL6, MDK, S100A12, TGFA;
w. HGF, IL6, LSP1, MDK, OSM;
x. CEACAM5, HGF, IL6, MDK, OSM;
y. IL6, LSP1, MDK, MMP12, TGFA;
z. IL6, MDK, MMP12, OSM, TGFA;
aa. CEACAM5, IL6, MDK, TGFA, WFDC2;
bb. CXCL9, IL6, LSP1, MDK, MMP12;
cc. IL6, LSP1, MDK, MMP12, OSM;
dd. IL6, KRT19, LSP1, MDK, TGFA;
ee. IL6, LSP1, MDK, TGFA, WFDC2;
ff. CEACAM5, IL6, LSP1, MDK, MMP12;
gg. CEACAM5, IL6, MDK, PLAUR, TGFA;
hh. HGF, IL6, MDK, TGFA; or
ii. IL6, MDK, TGFA, WFDC2.

18. The method of any one of claims 16-17, wherein a performance of the predictive model is characterized by an area under the curve (AUC) of at least 0.73.

19. The method of any one of claims 16-18, wherein a performance of the predictive model is characterized by a true positive rate of at least 30% at a false positive rate of 10%.

20. The method of any one of claims 1-6, wherein the plurality of biomarkers comprises IL6 and MDK and at least one more biomarker.

21. The method of claim 20, wherein the at least one more biomarker is selected from the group comprising: MMP12, LSP1, CEACAM5, HGF, OSM, and KRT19.

22. The method of any one of claims 20-21, wherein the plurality of biomarkers is selected from:
a. IL6, LSP1, MDK, MMP12;
b. CEACAM5, IL6, MDK, MMP12, TGFA;
c. HGF, IL6, MDK, MMP12, TGFA;
d. CEACAM5, IL6, MDK, TGFA;
e. IL6, MDK, MMP12, OSM;
f. IL6, MDK, MMP12, TGFA;
g. CEACAM5, IL6, LSP1, MDK, TGFA;
h. HGF, IL6, MDK, MMP12, OSM;
i. HGF, IL6, LSP1, MDK, MMP12; or
j. IL6, KRT19, MDK, MMP12, TGFA.

23. The method of any one of claims 20-22, wherein a performance of the predictive model is characterized by an area under the curve (AUC) of at least 0.74.

24. The method of any one of claims 20-23, wherein a performance of the predictive model is characterized by a true positive rate of at least 30% at a false positive rate of 10%.

25. The method of any one of claims 1-24, wherein the cancer is lung cancer.

26. The method of any one of claims 1-25, wherein the lung cancer is an adenocarcinoma, an adenosquamous cell cancer, a large cell cancer, a neuroendocrine cancer, a non-small cell lung cancer (NSCLC), a small cell cancer, or a squamous cell cancer.

27. The method of any one of claims 1-26, wherein the cancer is an early stage cancer.

28. The method of any one of claims 1-27, wherein the cancer is stage I, stage II, stage III, and/or stage IV lung cancer.

29. The method of any one of claims 1-28, wherein the expression levels of the plurality of biomarkers are determined from a test sample obtained from the subject.

30. The method of claim 29, wherein the test sample is a blood or serum sample.

31. The method of claim 29 or 30, wherein the subject is suspected of having an early stage cancer.

32. The method of claim 29 or 30, wherein the subject is not suspected of having an early stage cancer.

33. The method of any one of claims 1-32, wherein obtaining or having obtained the dataset comprises performing an assay to determine the expression levels of the plurality of biomarkers.

34. The method of claim 33, wherein the assay is a Proximity Extension Assay (PEA), a xMAP Multiplex Assay, a single molecule array (SIMOA) assay, mass spectrometry based protein or peptide assay, or an aptamer-based assay.

35. The method of claim 33 or 34, wherein performing the assay comprises contacting a test sample with a plurality of reagents comprising antibodies.

36. The method of claim 35, wherein the antibodies comprise one of monoclonal and polyclonal antibodies.

37. The method of claim 35, wherein the antibodies comprise both monoclonal and polyclonal antibodies.

38. The method of claim 1, wherein the method further comprises administering a treatment to the subject.

39. The method of claim 38, wherein the treatment comprises a surgery, a chemotherapy, a radiation therapy, a targeted therapy, immunotherapy, or any combination thereof.

40. A method for predicting presence or absence of a cancer in a subject, the method comprising:
at a computer system having one or more processors, and memory storing one or more programs for execution by the one or more processors:
a. obtaining, in electronic format, a dataset comprising expression levels of a plurality of biomarker from the subject, wherein the plurality of biomarkers comprises two or more biomarkers selected from: IL6, TGFA, S100A12, OSM, TFPI2, LSP1, MDK, CXCL9, CLEC4D, HGF, VWA1, CEACAM5, MMP12, KRT19, CASP8, WFDC2, and PLAUR; and
b. generating a prediction of presence or absence of the cancer in the subject by applying a predictive model to the expression levels of the plurality of biomarkers.

41. The method of claim 40, wherein a performance of the predictive model is characterized by an area under the curve (AUC) of at least 0.60, at least 0.61, at least 0.62, at least 0.63, at least 0.64, at least 0.65, at least 0.66, at least 0.67, at least 0.68, at least 0.69, at least 0.70, at least 0.71, at least 0.72, at least 0.73, or at least 0.74.

42. The method of any one of claims 40-41, wherein the performance of the predictive model is characterized by an area under the curve (AUC) of at least 0.60.

43. The method of any one of claims 40-42, wherein the performance of the predictive model is characterized by an area under the curve (AUC) of at least 0.74.

44. The method of any one of claims 40-43, wherein a performance metric of the predictive model is improved in comparison to a model solely incorporating CEACAM5.

45. The method of any one of claims 40-44, wherein the predictive model comprises a support vector machine (SVM) classifier.

46. The method of any one of claims 40-45, wherein the plurality of biomarkers comprises IL6 and at least one more biomarker.

47. The method of claim 46, wherein the at least one more biomarker is selected from the group comprising: TGFA, S100A12, OSM, TFPI2, LSP1, MDK, CXCL9, CLEC4D, HGF, VWA1, CEACAM5, MMP12, KRT19, CASP8, WFDC2, and PLAUR.

48. The method of any one of claims 46-47, wherein the plurality of biomarkers is selected from a combination of biomarkers as shown in Table 5.

49. The method of any one of claims 46-48, wherein a performance of the predictive model is characterized by an area under the curve (AUC) of at least 0.60.

50. The method of any one of claims 46-49, wherein a performance of the predictive model is characterized by a true positive rate of at least 30% at a false positive rate of 10%.

51. The method of any one of claims 40-45, wherein the plurality of biomarkers comprises IL6 and at least one more biomarker selected from the group comprising: TGFA, S100A12, OSM, TFPI2, LSP1, MDK, CXCL9, HGF, VWA1, CEACAM5, MMP12, KRT19, CASP8, WFDC2, and PLAUR.

52. The method of claim 51, wherein the plurality of biomarkers is selected from a combination of biomarkers as shown in Table 5.

53. The method of any one of claims 51-52, wherein a performance of the predictive model is characterized by an area under the curve (AUC) of at least 0.72.

54. The method of any one of claims 51-53, wherein a performance of the predictive model is characterized by a true positive rate of at least 30% at a false positive rate of 10%.

55. The method of any one of claims 40-45, wherein the plurality of biomarkers comprises IL6 and at least one more biomarker selected from the group comprising: TGFA, S100A12, OSM, LSP1, MDK, CXCL9, HGF, CEACAM5, MMP12, KRT19, WFDC2, and PLAUR.

56. The method of claim 55, wherein the plurality of biomarkers is selected from the group comprising:
a. IL6, LSP1, MDK, MMP12;
b. CEACAM5, IL6, MDK, MMP12, TGFA;
c. HGF, IL6, MDK, MMP12, TGFA;
d. CEACAM5, IL6, MDK, TGFA;
e. IL6, MDK, MMP12, OSM;
f. IL6, MDK, MMP12, TGFA;
g. CEACAM5, IL6, LSP1, MDK, TGFA;
h. HGF, IL6, MDK, MMP12, OSM;
i. HGF, IL6, LSP1, MDK, MMP12;
j. IL6, KRT19, MDK, MMP12, TGFA;
k. HGF, IL6, LSP1, MDK;
l. IL6, LSP1, MDK;
m. IL6, LSP1, MDK, TGFA;
n. IL6, MDK, TGFA;
o. CXCL9, IL6, LSP1, MDK;
p. CEACAM5, IL6, MDK, OSM, TGFA;
q. CEACAM5, HGF, IL6, MDK, TGFA;
r. CEACAM5, IL6, MDK, OSM;
s. CEACAM5, IL6, MDK, MMP12, OSM;
t. HGF, IL6, LSP1, MDK, TGFA;
u. CEACAM5, IL6, LSP1, MDK;
v. CEACAM5, IL6, MDK, S100A12, TGFA;
w. HGF, IL6, LSP1, MDK, OSM;
x. CEACAM5, HGF, IL6, MDK, OSM;
y. IL6, LSP1, MDK, MMP12, TGFA;
z. IL6, MDK, MMP12, OSM, TGFA;
aa. CEACAM5, IL6, MDK, TGFA, WFDC2;
bb. CXCL9, IL6, LSP1, MDK, MMP12;
cc. IL6, LSP1, MDK, MMP12, OSM;
dd. IL6, KRT19, LSP1, MDK, TGFA;
ee. IL6, LSP1, MDK, TGFA, WFDC2;
ff. CEACAM5, IL6, LSP1, MDK, MMP12;
gg. CEACAM5, IL6, MDK, PLAUR, TGFA;
hh. HGF, IL6, MDK, TGFA; or
ii. IL6, MDK, TGFA, WFDC2.

57. The method of any one of claims 55-56, wherein a performance of the predictive model is characterized by an area under the curve (AUC) of at least 0.73.

58. The method of any one of claims 55-57, wherein a performance of the predictive model is characterized by a true positive rate of at least 30% at a false positive rate of 10%.

59. The method of any one of claims 40-45, wherein the plurality of biomarkers comprises IL6 and MDK, and at least one more biomarker.

60. The method of claim 59, wherein the at least one more biomarker is selected from the group comprising: MMP12, LSP1, CEACAM5, HGF, OSM, and KRT19.

61. The method of any one of claims 59-60, wherein the plurality of biomarkers is selected from:
a. IL6, LSP1, MDK, MMP12;
b. CEACAM5, IL6, MDK, MMP12, TGFA;
c. HGF, IL6, MDK, MMP12, TGFA;
d. CEACAM5, IL6, MDK, TGFA;
e. IL6, MDK, MMP12, OSM;
f. IL6, MDK, MMP12, TGFA;
g. CEACAM5, IL6, LSP1, MDK, TGFA;
h. HGF, IL6, MDK, MMP12, OSM;
i. HGF, IL6, LSP1, MDK, MMP12; or
j. IL6, KRT19, MDK, MMP12, TGFA.

62. The method of any one of claims 59-61, wherein a performance of the predictive model is characterized by an area under the curve (AUC) of at least 0.74.

63. The method of any one of claims 59-62, wherein a performance of the predictive model is characterized by a true positive rate of at least 30% at a false positive rate of 10%.

64. The method of any one of claims 40-63, wherein the cancer is lung cancer.

65. The method of any one of claims 40-64, wherein the lung cancer is an adenocarcinoma, an adenosquamous cell cancer, a large cell cancer, a neuroendocrine cancer, a non-small cell lung cancer (NSCLC), a small cell cancer, or a squamous cell cancer.

66. The method of any one of claims 40-65, wherein the cancer is an early stage cancer.

67. The method of any one of claims 40-66, wherein the cancer is stage I, stage II, stage III, and/or stage IV lung cancer.

68. The method of any one of claims 40-67, wherein the expression levels of the plurality of biomarkers is determined from a test sample obtained from the subject.

69. The method of claim 68, wherein the test sample is a blood or serum sample.

70. The method of claim 68 or 69, wherein the subject is suspected of having an early stage cancer.

71. The method of claim 68 or 69, wherein the subject is not suspected of having an early stage cancer.

72. A non-transitory computer readable medium comprising instructions that, when executed by a processor, cause the processor to:
obtain a dataset comprising expression levels of a plurality of biomarkers from the subject, wherein the plurality of biomarkers comprises two or more biomarkers selected from: IL6, TGFA, S100A12, OSM, TFPI2, LSP1, MDK, CXCL9, CLEC4D, HGF, VWA1, CEACAM5, MMP12, KRT19, CASP8, WFDC2, and PLAUR; and
generate a prediction of presence or absence of the cancer in the subject by applying a predictive model to the expression levels of the plurality of biomarkers.

73. The non-transitory computer readable medium of claim 72, wherein a performance of the predictive model is characterized by an area under the curve (AUC) of at least 0.60, at least 0.61, at least 0.62, at least 0.63, at least 0.64, at least 0.65, at least 0.66, at least 0.67, at least 0.68, at least 0.69, at least 0.70, at least 0.71, at least 0.72, at least 0.73, or at least 0.74.

74. The non-transitory computer readable medium of any one of claims 72-73, wherein the performance of the predictive model is characterized by an area under the curve (AUC) of at least 0.60.

75. The non-transitory computer readable medium of any one of claims 72-74, wherein the performance of the predictive model is characterized by an area under the curve (AUC) of at least 0.74.

76. The non-transitory computer readable medium of any one of claims 72-75, wherein a performance metric of the predictive model is improved in comparison to a model solely incorporating CEACAM5.

77. The non-transitory computer readable medium of any one of claims 72-76, wherein the predictive model comprises a support vector machine (SVM) classifier.

78. The non-transitory computer readable medium of any one of claims 72-77, wherein the plurality of biomarkers comprises IL6 and at least one more biomarker.

79. The non-transitory computer readable medium of claim 78, wherein the at least one more biomarker is selected from the group comprising: TGFA, S100A12, OSM, TFPI2, LSP1, MDK, CXCL9, CLEC4D, HGF, VWA1, CEACAM5, MMP12, KRT19, CASP8, WFDC2, and PLAUR.

80. The non-transitory computer readable medium of any one of claims 78-79, wherein the plurality of biomarkers is selected from a combination of biomarkers as shown in Table 5.

81. The non-transitory computer readable medium of any one of claims 78-80, wherein a performance of the predictive model is characterized by an area under the curve (AUC) of at least 0.60.

82. The non-transitory computer readable medium of any one of claims 78-81, wherein a performance of the predictive model is characterized by a true positive rate of at least 30% at a false positive rate of 10%.

83. The non-transitory computer readable medium of any one of claims 72-77, wherein the plurality of biomarkers comprises IL6 and at least one more biomarker selected from the group comprising: TGFA, S100A12, OSM, TFPI2, LSP1, MDK, CXCL9, HGF, VWA1, CEACAM5, MMP12, KRT19, CASP8, WFDC2, and PLAUR.

84. The non-transitory computer readable medium of claim 83, wherein the plurality of biomarkers is selected from a combination of biomarkers as shown in Table 5.

85. The non-transitory computer readable medium of any one of claims 83-84, wherein a performance of the predictive model is characterized by an area under the curve (AUC) of at least 0.72.

86. The non-transitory computer readable medium of any one of claims 83-85, wherein a performance of the predictive model is characterized by a true positive rate of at least 30% at a false positive rate of 10%.

87. The non-transitory computer readable medium of any one of claims 72-77, wherein the plurality of biomarkers comprises IL6 and at least one more biomarker selected from the group comprising: TGFA, S100A12, OSM, LSP1, MDK, CXCL9, HGF, CEACAM5, MMP12, KRT19, WFDC2, and PLAUR.

88. The non-transitory computer readable medium of claim 87, wherein the plurality of biomarkers is selected from the group comprising:
a. IL6, LSP1, MDK, MMP12;
b. CEACAM5, IL6, MDK, MMP12, TGFA;
c. HGF, IL6, MDK, MMP12, TGFA;
d. CEACAM5, IL6, MDK, TGFA;
e. IL6, MDK, MMP12, OSM;
f. IL6, MDK, MMP12, TGFA;
g. CEACAM5, IL6, LSP1, MDK, TGFA;
h. HGF, IL6, MDK, MMP12, OSM;
i. HGF, IL6, LSP1, MDK, MMP12;
j. IL6, KRT19, MDK, MMP12, TGFA;
k. HGF, IL6, LSP1, MDK;
l. IL6, LSP1, MDK;
m. IL6, LSP1, MDK, TGFA;
n. IL6, MDK, TGFA;
o. CXCL9, IL6, LSP1, MDK;
p. CEACAM5, IL6, MDK, OSM, TGFA;
q. CEACAM5, HGF, IL6, MDK, TGFA;
r. CEACAM5, IL6, MDK, OSM;
s. CEACAM5, IL6, MDK, MMP12, OSM;
t. HGF, IL6, LSP1, MDK, TGFA;
u. CEACAM5, IL6, LSP1, MDK;
v. CEACAM5, IL6, MDK, S100A12, TGFA;
w. HGF, IL6, LSP1, MDK, OSM;
x. CEACAM5, HGF, IL6, MDK, OSM;
y. IL6, LSP1, MDK, MMP12, TGFA;
z. IL6, MDK, MMP12, OSM, TGFA;
aa. CEACAM5, IL6, MDK, TGFA, WFDC2;
bb. CXCL9, IL6, LSP1, MDK, MMP12;
cc. IL6, LSP1, MDK, MMP12, OSM;
dd. IL6, KRT19, LSP1, MDK, TGFA;
ee. IL6, LSP1, MDK, TGFA, WFDC2;
ff. CEACAM5, IL6, LSP1, MDK, MMP12;
gg. CEACAM5, IL6, MDK, PLAUR, TGFA;
hh. HGF, IL6, MDK, TGFA; or
ii. IL6, MDK, TGFA, WFDC2.

89. The non-transitory computer readable medium of any one of claims 87-88, wherein a performance of the predictive model is characterized by an area under the curve (AUC) of at least 0.73.

90. The non-transitory computer readable medium of any one of claims 87-89, wherein a performance of the predictive model is characterized by a true positive rate of at least 30% at a false positive rate of 10%.

91. The non-transitory computer readable medium of any one of claims 72-77, wherein the plurality of biomarkers comprises IL6 and MDK, and at least one more biomarker.

92. The non-transitory computer readable medium of claim 91, wherein the at least one more biomarker is selected from the group comprising: MMP12, LSP1, CEACAM5, HGF, OSM, and KRT19.

93. The non-transitory computer readable medium of any one of claims 91-92, wherein the plurality of biomarkers is selected from:
a. IL6, LSP1, MDK, MMP12;
b. CEACAM5, IL6, MDK, MMP12, TGFA;
c. HGF, IL6, MDK, MMP12, TGFA;
d. CEACAM5, IL6, MDK, TGFA;
e. IL6, MDK, MMP12, OSM;
f. IL6, MDK, MMP12, TGFA;
g. CEACAM5, IL6, LSP1, MDK, TGFA;
h. HGF, IL6, MDK, MMP12, OSM;
i. HGF, IL6, LSP1, MDK, MMP12; or
j. IL6, KRT19, MDK, MMP12, TGFA.

94. The non-transitory computer readable medium of any one of claims 91-93, wherein a performance of the predictive model is characterized by an area under the curve (AUC) of at least 0.74.

95. The non-transitory computer readable medium of any one of claims 91-94, wherein a performance of the predictive model is characterized by a true positive rate of at least 30% at a false positive rate of 10%.

96. The non-transitory computer readable medium of any one of claims 72-95, wherein the cancer is lung cancer.

97. The non-transitory computer readable medium of any one of claims 72-96, wherein the lung cancer is an adenocarcinoma, an adenosquamous cell cancer, a large cell cancer, a neuroendocrine cancer, a non-small cell lung cancer (NSCLC), a small cell cancer, or a squamous cell cancer.

98. The non-transitory computer readable medium of any one of claims 72-97, wherein the cancer is an early stage cancer.

99. The non-transitory computer readable medium of any one of claims 72-98, wherein the cancer is stage I, stage II, stage III, and/or stage IV lung cancer.

100. The non-transitory computer readable medium of any one of claims 72-99, wherein the expression levels of the plurality of biomarkers is determined from a test sample obtained from the subject.

101. The non-transitory computer readable medium of claim 100, wherein the test sample is a blood or serum sample.

102. The non-transitory computer readable medium of claim 100 or 101, wherein the subject is suspected of having an early stage cancer.

103. The non-transitory computer readable medium of claim 100 or 101, wherein the subject is not suspected of having an early stage cancer.

104. A system comprising:
a set of reagents used for determining expression levels for a plurality of biomarkers from a test sample from the subject, wherein the plurality of biomarkers comprises two or more biomarkers selected from: IL6, TGFA, S100A12, OSM, TFPI2, LSP1, MDK, CXCL9, CLEC4D, HGF, VWA1, CEACAM5, MMP12, KRT19, CASP8, WFDC2, and PLAUR;
an apparatus configured to receive a mixture of one or more reagents in the set and the test sample and to measure the expression levels for the biomarkers from the test sample; and
a computer system communicatively coupled to the apparatus to obtain a dataset comprising the expression levels for the plurality of biomarkers from the test sample and to generate a presence or absence of cancer in the subject by applying a predictive model to the expression levels of the plurality of biomarkers.

105. The system of claim 104, wherein a performance of the predictive model is characterized by an area under the curve (AUC) of at least 0.60, at least 0.61, at least 0.62, at least 0.63, at least 0.64, at least 0.65, at least 0.66, at least 0.67, at least 0.68, at least 0.69, at least 0.70, at least 0.71, at least 0.72, at least 0.73, or at least 0.74.

106. The system of any one of claims 104-105, wherein a performance of the predictive model is characterized by an area under the curve (AUC) of at least 0.60.

107. The system of any one of claims 104-106, wherein a performance of the predictive model is characterized by an area under the curve (AUC) of at least 0.74.

108. The system of any one of claims 104-107, wherein a performance metric of the predictive model is improved in comparison to a model solely incorporating CEA.

109. The system of any one of claims 104-108, wherein the predictive model comprises a support vector machine (SVM) classifier.

110. The system of any one of claims 104-109, wherein the plurality of biomarkers comprises IL6 and at least one more biomarker.

111. The system of claim 110, wherein the at least one more biomarker is selected from the group comprising: TGFA, S100A12, OSM, TFPI2, LSP1, MDK, CXCL9, CLEC4D, HGF, VWA1, CEACAM5, MMP12, KRT19, CASP8, WFDC2, and PLAUR.

112. The system of any one of claims 110-111, wherein the plurality of biomarkers is selected from a combination of biomarkers as shown in Table 5.

113. The system of any one of claims 110-112, wherein a performance of the predictive model is characterized by an area under the curve (AUC) of at least 0.60.

114. The system of any one of claims 110-113, wherein a performance of the predictive model is characterized by a true positive rate of at least 30% at a false positive rate of 10%.

115. The system of any one of claims 104-109, wherein the plurality of biomarkers comprises IL6 and at least one more biomarker selected from the group comprising: TGFA, S100A12, OSM, TFPI2, LSP1, MDK, CXCL9, HGF, VWA1, CEACAM5, MMP12, KRT19, CASP8, WFDC2, and PLAUR.

116. The system of claim 115, wherein the plurality of biomarkers is selected from a combination of biomarkers as shown in Table 5.

117. The system of any one of claims 115-116, wherein a performance of the predictive model is characterized by an area under the curve (AUC) of at least 0.72.

118. The system of any one of claims 115-117, wherein a performance of the predictive model is characterized by a true positive rate of at least 30% at a false positive rate of 10%.

119. The system of any one of claims 104-109, wherein the plurality of biomarkers comprises IL6 and at least one more biomarker selected from the group comprising: TGFA, S100A12, OSM, LSP1, MDK, CXCL9, HGF, CEACAM5, MMP12, KRT19, WFDC2, and PLAUR.

120. The system of claim 119, wherein the plurality of biomarkers is selected from the group comprising:
a. IL6, LSP1, MDK, MMP12;
b. CEACAM5, IL6, MDK, MMP12, TGFA;
c. HGF, IL6, MDK, MMP12, TGFA;
d. CEACAM5, IL6, MDK, TGFA;
e. IL6, MDK, MMP12, OSM;
f. IL6, MDK, MMP12, TGFA;
g. CEACAM5, IL6, LSP1, MDK, TGFA;
h. HGF, IL6, MDK, MMP12, OSM;
i. HGF, IL6, LSP1, MDK, MMP12;
j. IL6, KRT19, MDK, MMP12, TGFA;
k. HGF, IL6, LSP1, MDK;
l. IL6, LSP1, MDK;
m. IL6, LSP1, MDK, TGFA;
n. IL6, MDK, TGFA;
o. CXCL9, IL6, LSP1, MDK;
p. CEACAM5, IL6, MDK, OSM, TGFA;
q. CEACAM5, HGF, IL6, MDK, TGFA;
r. CEACAM5, IL6, MDK, OSM;
s. CEACAM5, IL6, MDK, MMP12, OSM;
t. HGF, IL6, LSP1, MDK, TGFA;
u. CEACAM5, IL6, LSP1, MDK;
v. CEACAM5, IL6, MDK, S100A12, TGFA;
w. HGF, IL6, LSP1, MDK, OSM;
x. CEACAM5, HGF, IL6, MDK, OSM;
y. IL6, LSP1, MDK, MMP12, TGFA;
z. IL6, MDK, MMP12, OSM, TGFA;
aa. CEACAM5, IL6, MDK, TGFA, WFDC2;
bb. CXCL9, IL6, LSP1, MDK, MMP12;
cc. IL6, LSP1, MDK, MMP12, OSM;
dd. IL6, KRT19, LSP1, MDK, TGFA;
ee. IL6, LSP1, MDK, TGFA, WFDC2;
ff. CEACAM5, IL6, LSP1, MDK, MMP12;
gg. CEACAM5, IL6, MDK, PLAUR, TGFA;
hh. HGF, IL6, MDK, TGFA; or
ii. IL6, MDK, TGFA, WFDC2.

121. The system of any one of claims 119-120, wherein a performance of the predictive model is characterized by an area under the curve (AUC) of at least 0.73.

122. The system of any one of claims 119-121, wherein a performance of the predictive model is characterized by a true positive rate of at least 30% at a false positive rate of 10%

123. The system of any one of claims 104-109, wherein the plurality of biomarkers comprises IL6 and MDK, and at least one more biomarker.

124. The system of claim 123, wherein the at least one more biomarker is selected from the group comprising: MMP12, LSP1, CEACAM5, HGF, OSM, and KRT19.

125. The system of any one of claims 123-124, wherein the plurality of biomarkers is selected from:
a. IL6, LSP1, MDK, MMP12;
b. CEACAM5, IL6, MDK, MMP12, TGFA;
c. HGF, IL6, MDK, MMP12, TGFA;
d. CEACAM5, IL6, MDK, TGFA;
e. IL6, MDK, MMP12, OSM;
f. IL6, MDK, MMP12, TGFA;
g. CEACAM5, IL6, LSP1, MDK, TGFA;
h. HGF, IL6, MDK, MMP12, OSM;
i. HGF, IL6, LSP1, MDK, MMP12; or
j. IL6, KRT19, MDK, MMP12, TGFA.

126. The system of any one of claims 123-125, wherein a performance of the predictive model is characterized by an area under the curve (AUC) of at least 0.74.

127. The system of any one of claims 123-126, wherein a performance of the predictive model is characterized by a true positive rate of at least 30% at a false positive rate of 10%.

128. The system of any one of claims 104-127, wherein the cancer is lung cancer.

129. The system of any one of claims 104-128, wherein the lung cancer is an adenocarcinoma, an adenosquamous cell cancer, a large cell cancer, a neuroendocrine cancer, a non-small cell lung cancer (NSCLC), a small cell cancer, or a squamous cell cancer.

130. The system of any one of claims 104-129, wherein the cancer is an early stage cancer.

131. The system of any one of claims 104-130, wherein the cancer is stage I, stage II, stage III, and/or stage IV lung cancer.

132. The system of any one of claims 104-131, wherein the expression levels of the plurality of biomarkers is determined from a test sample obtained from the subject.

133. The system of claim 132, wherein the test sample is a blood or serum sample.

134. The system of claim 132 or 133, wherein the subject is suspected of having an early stage cancer.

135. The system of claim 132 or 133, wherein the subject is not suspected of having an early stage cancer.

136. A kit for predicting presence or absence of cancer in a subject, the kit comprising:
a set of reagents for determining expression levels for a plurality of biomarkers from a test sample from the subject, wherein the plurality of biomarkers comprises two or more biomarkers selected from: IL6, TGFA, S100A12, OSM, TFPI2, LSP1, MDK, CXCL9, CLEC4D, HGF, VWA1, CEACAM5, MMP12, KRT19, CASP8, WFDC2, and PLAUR; and
instructions for using the set of reagents to determine the expression levels of the plurality of biomarkers from the test sample and to generate a prediction of presence or absence of cancer in the subject by applying a predictive model to the expression levels of the plurality of biomarkers.

137. The kit of claim 136, wherein a performance of the predictive model is characterized by an area under the curve (AUC) of at least 0.60, at least 0.61, at least 0.62, at least 0.63, at least 0.64, at least 0.65, at least 0.66, at least 0.67, at least 0.68, at least 0.69, at least 0.70, at least 0.71, at least 0.72, at least 0.73, or at least 0.74.

138. The kit of any one of claims 136-137, wherein the performance of the predictive model is characterized by an area under the curve (AUC) of at least 0.60.

139. The kit of any one of claims 136-138, wherein the performance of the predictive model is characterized by an area under the curve (AUC) of at least 0.74.

140. The kit of any one of claims 136-139, wherein a performance metric of the predictive model is improved in comparison to a model solely incorporating CEACAM5.

141. The kit of any one of claims 136-140, wherein the predictive model comprises a support vector machine (SVM) classifier.

142. The kit of any one of claims 136-141, wherein the plurality of biomarkers comprises IL6 and at least one more biomarker.

143. The kit of claim 142, wherein the at least one more biomarker is selected from the group comprising: TGFA, S100A12, OSM, TFPI2, LSP1, MDK, CXCL9, CLEC4D, HGF, VWA1, CEACAM5, MMP12, KRT19, CASP8, WFDC2, and PLAUR.

144. The kit of any one of claims 141-143, wherein the plurality of biomarkers is selected from a combination of biomarkers as shown in Table 5.

145. The kit of any one of claims 141-144, wherein a performance of the predictive model is characterized by an area under the curve (AUC) of at least 0.60.

146. The kit of any one of claims 141-145, wherein a performance of the predictive model is characterized by a true positive rate of at least 30% at a false positive rate of 10%.

147. The kit of any one of claims 136-141, wherein the plurality of biomarkers comprises IL6 and at least one more biomarker selected from the group comprising: TGFA, S100A12, OSM, TFPI2, LSP1, MDK, CXCL9, HGF, VWA1, CEACAM5, MMP12, KRT19, CASP8, WFDC2, and PLAUR.

148. The kit of claim 147, wherein the plurality of biomarkers is selected from a combination of biomarkers as shown in Table 5.

149. The kit of any one of claims 147-148, wherein a performance of the predictive model is characterized by an area under the curve (AUC) of at least 0.72.

150. The kit of any one of claims 147-149, wherein a performance of the predictive model is characterized by a true positive rate of at least 30% at a false positive rate of 10%.

151. The kit of any one of claims 136-141, wherein the plurality of biomarkers comprises IL6 and at least one more biomarker selected from the group comprising: TGFA, S100A12, OSM, LSP1, MDK, CXCL9, HGF, CEACAM5, MMP12, KRT19, WFDC2, and PLAUR.

152. The kit of claim 151, wherein the plurality of biomarkers is selected from the group comprising:
a. IL6, LSP1, MDK, MMP12;
b. CEACAM5, IL6, MDK, MMP12, TGFA;
c. HGF, IL6, MDK, MMP12, TGFA;
d. CEACAM5, IL6, MDK, TGFA;
e. IL6, MDK, MMP12, OSM;
f. IL6, MDK, MMP12, TGFA;
g. CEACAM5, IL6, LSP1, MDK, TGFA;
h. HGF, IL6, MDK, MMP12, OSM;
i. HGF, IL6, LSP1, MDK, MMP12;
j. IL6, KRT19, MDK, MMP12, TGFA;
k. HGF, IL6, LSP1, MDK;
l. IL6, LSP1, MDK;
m. IL6, LSP1, MDK, TGFA;
n. IL6, MDK, TGFA;
o. CXCL9, IL6, LSP1, MDK;
p. CEACAM5, IL6, MDK, OSM, TGFA;
q. CEACAM5, HGF, IL6, MDK, TGFA;
r. CEACAM5, IL6, MDK, OSM;
s. CEACAM5, IL6, MDK, MMP12, OSM;
t. HGF, IL6, LSP1, MDK, TGFA;
u. CEACAM5, IL6, LSP1, MDK;
v. CEACAM5, IL6, MDK, S100A12, TGFA;
w. HGF, IL6, LSP1, MDK, OSM;
x. CEACAM5, HGF, IL6, MDK, OSM;
y. IL6, LSP1, MDK, MMP12, TGFA;
z. IL6, MDK, MMP12, OSM, TGFA;
aa. CEACAM5, IL6, MDK, TGFA, WFDC2;
bb. CXCL9, IL6, LSP1, MDK, MMP12;
cc. IL6, LSP1, MDK, MMP12, OSM;
dd. IL6, KRT19, LSP1, MDK, TGFA;
ee. IL6, LSP1, MDK, TGFA, WFDC2;
ff. CEACAM5, IL6, LSP1, MDK, MMP12;
gg. CEACAM5, IL6, MDK, PLAUR, TGFA;
hh. HGF, IL6, MDK, TGFA; or
ii. IL6, MDK, TGFA, WFDC2.

153. The kit of any one of claims 151-152, wherein a performance of the predictive model is characterized by an area under the curve (AUC) of at least 0.73.

154. The kit of any one of claims 151-153, wherein a performance of the predictive model is characterized by a true positive rate of at least 30% at a false positive rate of 10%.

155. The kit of any one of claims 136-141, wherein the plurality of biomarkers comprises IL6 and MDK, and at least one more biomarker.

156. The kit of claim 155, wherein the at least one more biomarker is selected from the group comprising: MMP12, LSP1, CEACAM5, HGF, OSM, and KRT19.

157. The kit of any one of claims 155-156, wherein the plurality of biomarkers is selected from:
a. IL6, LSP1, MDK, MMP12;
b. CEACAM5, IL6, MDK, MMP12, TGFA;
c. HGF, IL6, MDK, MMP12, TGFA;
d. CEACAM5, IL6, MDK, TGFA;
e. IL6, MDK, MMP12, OSM;
f. IL6, MDK, MMP12, TGFA;
g. CEACAM5, IL6, LSP1, MDK, TGFA;
h. HGF, IL6, MDK, MMP12, OSM;
i. HGF, IL6, LSP1, MDK, MMP12; or
j. IL6, KRT19, MDK, MMP12, TGFA.

158. The kit of any one of claims 155-157, wherein a performance of the predictive model is characterized by an area under the curve (AUC) of at least 0.74.

159. The kit of any one of claims 155-158, wherein a performance of the predictive model is characterized by a true positive rate of at least 30% at a false positive rate of 10%

160. The kit of any one of claims 136-159, wherein the cancer is lung cancer.

161. The kit of any one of claims 136-160, wherein the lung cancer is an adenocarcinoma, an adenosquamous cell cancer, a large cell cancer, a neuroendocrine cancer, a non-small cell lung cancer (NSCLC), a small cell cancer, or a squamous cell cancer.

162. The kit of any one of claims 136-161, wherein the cancer is an early stage cancer.

163. The kit of any one of claims 136-162, wherein the cancer is stage I, stage II, stage III, and/or stage IV lung cancer.

164. The kit of any one of claims 136-163, wherein the expression levels of the plurality of biomarkers is determined from a test sample obtained from the subject.

165. The kit of claim 164, wherein the test sample is a blood or serum sample.

166. The kit of claim 164 or 165, wherein the subject is suspected of having an early stage cancer.

167. The kit of claim 164 or 165, wherein the subject is not suspected of having an early stage cancer.

168. The kit of any one of claims 136-167, wherein the set of reagents is used to perform an assay to determine the expression levels of the plurality of biomarkers.

169. The kit of claim 168, wherein the assay is a Proximity Extension Assay (PEA), a xMAP Multiplex Assay, a single molecule array (SIMOA) assay, mass spectrometry based protein or peptide assay, or an aptamer-based assay.

170. The kit of claim 168 or 169, wherein performing the assay comprises contacting a test sample with a plurality of reagents comprising antibodies.

171. The kit of claim 170, wherein the antibodies comprise one of monoclonal and polyclonal antibodies.

172. The kit of claim 170, wherein the antibodies comprise both monoclonal and polyclonal antibodies.
